(19) United States
(12) Patent Application Publication
(10) Pub. No.: US 2024/0131183 A1
(43) Pub. Date: Apr. 25, 2024

(54) COMPOSITIONS AND METHODS FOR NANOPARTICLE SEED SUBSTRATES

(71) Applicant: Ligandal, Inc., San Franciso, CA (US)

(72) Inventor: Andre Watson, San Francisco, CA (US)

(21) Appl. No.: 18/165,840

(22) Filed: Feb. 7, 2023

Related U.S. Application Data
(63) Continuation of application No. PCT/US2021/047021, filed on Aug. 20, 2021.
(60) Provisional application No. 63/068,931, filed on Aug. 21, 2020.

(51) Int. Cl. A61K 47/69; A61K 47/64; G16C 60/00

(52) U.S. Cl. CPC A61K 47/6931; A61K 47/64; G16C 60/00

(57) ABSTRACT

Provided herein are certain compositions and methods for nanoparticle seed substrates for self-assimilable nanoparticles and methods for optimized design of the same.

### PRIORITY

This application is a continuation of International Application No. PCT/US2021/047021, filed on Aug. 20, 2021, which claims the benefit of U.S. Provisional Application No. 63/068,931, filed Aug. 21, 2020, the disclosures of which are incorporated herein by reference in their entirety, including drawings.

### SEQUENCE LISTING

This application contains an ST.26 compliant Sequence Listing, which is submitted concurrently in xml format via EFS-Web or Patent Center and is hereby incorporated by reference in its entirety. The .xml copy, created on Sep. 22, 2023 is named Replacement Ligandal 134554-8001 US02 Sequence Listing.xml and is 116,000 bytes in size.

### SUMMARY

In some embodiments, compositions provided may include a nanoparticle seed substrate having a three-dimensional surface comprising a plurality of binding patches and a plurality of moieties coupled to the plurality of binding patches, wherein the combination of coupled moieties inhibits nanoparticle seed substrate aggregation by coupling of the first nanoparticle seed substrate to a substrate.

In certain aspects, the nanoparticle seed substrate is a non-proteinaceous nanoparticle seed substrate. In certain aspects, the non-proteinaceous nanoparticle seed substrate is an electrostatic, lipidic, gold, or metallic particle.

In some embodiments, compositions provided may include a nanoparticle seed substrate having a zwitterionic three-dimensional charge-tunable surface comprising a plurality of binding patches, and a plurality of moieties coupled to the plurality of binding patches, wherein the combination of coupled moieties inhibits nanoparticle seed substrate aggregation by electrostatic coupling of the nanoparticle seed substrate to a substrate.

In some embodiments, compositions may include a plurality of binding patches comprising a plurality of cationic and a plurality of anionic binding patches, and a nanoparticle seed substrate having a zwitterionic charge ratio, wherein the zwitterionic charge ratio is defined by the number of the cationic binding patches to the number of anionic binding patches (+/−) and the charge ratio is modified compared to an unmodified seed substrate.

In some embodiments, compositions provided herein further comprise at least one moiety comprising a modified protein. In some embodiments, the modified protein may comprise an anchor, wherein the anchor is designed to interact with a binding patch and wherein the interaction between the anchor and the binding patch may be hydrophilic, hydrophobic, electrostatic, covalent, or non-covalent. In some embodiments, the modified protein may comprise at least one linker, wherein at least one linker is coupled to the anchor; and a payload coupled to at least one linker, or a functional domain coupled to at least one linker.

In some embodiments, compositions provided herein may comprise a plurality of cationic and a plurality of anionic binding patches. In certain embodiments, a nanoparticle seed substrate may have a zwitterionic charge ratio, wherein the zwitterionic charge ratio is defined by the number of the cationic binding patches to the number of anionic binding patches (+/−) and the charge ratio is modified compared to an unmodified seed substrate.

In some embodiments, compositions provided herein may comprise a plurality of negatively charged moieties electrostatically bound to cationic binding patches, or a plurality of positively charged moieties electrostatically bound to anionic binding patches.

In some embodiments, compositions provided herein may comprise a plurality of negatively charged moieties electrostatically bound to cationic binding patches, and a plurality of positively charged moieties electrostatically bound to anionic binding patches.

In certain embodiments, compositions provided herein may further comprise at least one moiety, wherein the moiety is a polymeric molecule.

In certain embodiments, compositions provided herein may comprise a polymeric molecule selected from a gRNA molecule, a donor DNA molecule, an mRNA molecule, an siRNA molecule, a dsRNA molecule, an aptamer, a charge-switchable polymer, a bioreducable polymer, a glycosaminoglycan (GAG), an oligosaccharide, a proteoglycan, an anionic peptide sequence, an anionic glycopeptide sequence, a sphingolipid, sphingosine-1-phosphate, a ceramide, a ganglioside, a lipid, an anionic lipid, a cationic lipid, an anionic polymer, alginate, an agmatine-rich sequence, an arginine-rich sequence, a histidine-rich sequence, a lysine-rich sequence, a citrulline-rich sequence, an ornithine-rich sequence, gelatin, a carboxylate-rich polymer, a phosphate-rich polymer, a sulfate-rich polymer, a peptoid, a polysaccharide, a poly(aspartic acid) rich sequence, a poly(glutamic acid) rich sequence, a branched polymer or co-polymer variant thereof, or a dendrimeric polymer or co-polymer variant thereof, p(asp)[DET], an amine-rich polymer, a charge-modified polymeric backbone, an anionic charge-modified polymer backbone, a cationic charge-modified polymer backbone, poly(β-amino esters), a negatively charge-functionalized poly(β-amino ester), a positively charge-functionalized poly(β-amino ester), a lipid, a cationic lipid, an anionic lipid, a cell penetrating peptide, a histone-derived sequence, an NLS-derived sequence, a subcellular-localizing sequence, a subcellular-functional sequence, a DNA-binding protein, an RNA-binding protein, an anchor-linker-ligand complex, an anchor-ligand complex, an anchor-functional domain complex, an anchor-linker-functional domain complex, a cationic charge-modified polymer backbone or a co-polymer variant thereof, or a multi-domain polymer.

In some embodiments, compositions provided herein may comprise a polymeric molecule that is a fusion polymer. In certain embodiments, the fusion polymer may comprise a polymeric molecule, a stereoisomer of a polymeric molecule, or a polymeric molecule comprising an additional moiety as a contiguous portion of its sequence.

In certain embodiments, compositions provided herein comprising a fusion polymer including a polymeric molecule comprising an additional moiety as a contiguous portion of its sequence may comprise an additional moiety selected from a functional domain, a payload domain, polyethylene glycol (PEG), N-(2-Hydroxypropyl) methacrylamide (HPMA), poly(sarcosine), a biocompatible linker or terminal polymer sequence assisting in forming phase-separation, a gRNA molecule, a donor DNA molecule, an mRNA molecule, an siRNA molecule, an miRNA molecule, a dsRNA molecule, an aptamer, a glycosaminoglycan (GAG), an oligosaccharide, a proteoglycan, an anionic peptide sequence, an anionic glycopeptide sequence, a sphingolipid, sphingosine-1-phosphate, a ceramide, a ganglioside, an anionic lipid, an anionic polymer, alginate, gelatin, a carboxylate-rich polymer, a phosphate-rich polymer, a sulfate-rich polymer, a peptoid, a negatively charge-functionalized poly(β-amino ester), a polysaccharide, a poly(aspartic acid) rich sequence, a poly(glutamic acid) rich sequence, agmatine, a charge-modified polymer backbone, an anionic charge-modified polymer backbone, a cationic charge-modified polymer backbone, a branched polymer backbone or a co-polymer variant thereof, a dendrimeric polymer backbone or a co-polymer variant thereof, or a charge-switchable polymer.

In certain embodiments, compositions provided herein may comprise a multi-domain polymer comprising a plurality of domains selected from cationic domains, anionic domains, and neutral domains.

In some embodiments, compositions provided herein may include a functional domain comprising one or more of the following: a ligand, an endosomolytic domain, a subcellular functional domain, a subcellular trafficking domain, a histone-mimetic domain, a nuclear-material-mimetic domain, an environmental-specific unpackaging domain, a protein-corona-inhibitory domain, a macrophage-endocytosis-inhibitory domain, a receptor agonist domain, a receptor antagonist domain, a receptor partial agonist domain, a β-arrestin-biased agonist domain, Gs-biased agonist, Gi-biased agonist, Gq-biased agonist, a caveolae-mediated endocytosis trigger, a clathrin-mediated endocytosis trigger, a lysosomal trigger, a late endosome trigger, a “long recycling” endosome trigger, a “short recycling” endosome trigger, an early endosome trigger, a Rab-mimetic endosomal sorting protein, a kinesin-binding domain, a dynein-binding domain, a biomimetic domain, a cell-mimetic domain, polyethylene glycol (PEG), poly(sarcosine), a N-(2-Hydroxypropyl) (HPMA) linker, a HPMA terminal sequence, a biodegradable polymer, an endosomolytic peptide sequence, a viral peptide sequence, a virally-derived subcellular sorting sequence, a nuclear trafficking sequence, a microtubule-binding sequence, a histone-derived sequence, a TLR-binding molecule, or a subcellular trafficking sequence.

In some embodiments, compositions provided herein may include a functional domain comprising a cell-targeting motif.

In some embodiments, cell-targeting motifs may be selected from an antibody, a single-chain variable fragment (ScFv), an aptamer, a peptoid, a polymer, a lipid, a polysaccharide, a subcellular cell-targeting motif, an extracellular cell-targeting motif, or a multi-domain sequence.

In some embodiments, compositions provided herein may include a functional domain comprising a cell-penetrating motif.

In some embodiments, cell-penetrating motifs provided herein may be selected from p(asp)[DETm], a cationic polymer, poly(L-arginine) (PLR), poly(L-lysine) (PLK), poly(L-ornithine) (PLO), poly(L-citrulline) (PLCIT), a cationic-rich sequence, a histone, or a cell penetrating peptide (CPP). CPPs may include, for example, those provided in U.S. Provisional Appl. No. 62/685,240, Rodrigues 2015, Lee 2012, or Vandenberg 1991.

In some embodiments, compositions provided herein may further comprise at least one moiety comprising a modified protein. The modified protein may comprise an anchor, wherein the anchor is designed to interact with a binding patch and wherein the interaction between the anchor and the binding patch may be hydrophilic, hydrophobic, electrostatic, covalent, or non-covalent. The modified protein may additionally comprise at least one linker, wherein at least one linker is coupled to the anchor.

In some embodiments, linkers provided herein may be a terminal linker.

In some embodiments, anchors provided herein may be selected from a cationic anchor or an anionic anchor.

In some embodiments, linkers provided herein may be selected from polyethylene glycol (PEG), N-(2-Hydroxypropyl) methacrylamide (HPMA), poly(sarcosine), a poly(hydrophilic) polymer, a poly(hydrophobic) polymer, a poly(charged) polymer, a charge-switching polymer, a rigid domain, flexible domain, or an aliphatic domain.

In some embodiments, linkers provided herein may be a multi-domain linker, and at least one domain may be selected from polyethylene glycol (PEG), N-(2-Hydroxypropyl) methacrylamide (HPMA), poly(sarcosine), a poly(hydrophilic) polymer, a poly(hydrophobic) polymer, a poly(charged) polymer, a charge-switching polymer, a rigid domain, flexible domain, or an aliphatic domain.

In some embodiments, compositions provided herein may comprise a nanoparticle seed substrate wherein the seed substrate comprises a protein.

In some embodiments, compositions provided herein may comprise a nanoparticle seed substrate, wherein the seed substrate comprises a protein that may be selected from a peptide-sequence-guided nuclease, a peptide-sequence-guided transposon, RNA-guided nuclease, an RNA-guided transposon, a DNA-guided nuclease, a DNA-guided transposon, a synthetic PNA/MNA/LNA/modRNA-guided nuclease, a synthetic PNA/MNA/LNA/modRNA-guided transposon, a DNA-repair enhancing protein, Cas9, CasX, CasY, Cpf1, Cas13, MAD7, Rad51, Rad54, transcription activator-like (TAL) effectors (TALEs), TALE nucleases (TALENs), zinc-finger proteins (ZFPs), zinc-finger nucleases (ZFNs), DNA-guided polypeptides, Natronobacterium gregoryi Argonaute (NgAgo), transposons, piggyBac, sleeping beauty, Tc1/mariner, Tol2, PIF/harbinger, hAT, mutator, merlin, transib, helitron, maverick, frog prince, minos, Himar1, meganucleases, I-SceI, I-CeuI, I-CreI, I-DmoI, I-ChuI, I-DirI, I-FImuI, I-FImuII, I-AniI, I-SceIV, I-CsmI, I-PanI, I-PanII, I-PanMI, I-ScelI, I-PpoI, I-SceIII, I-LtrI, I-GpiI, I-GZeI, I-OnuI, I-HjeMI, I-MsoI, I-TevI, I-TevlI, I-TevIII, PI-MleI, PI-MtuI, PI-PspI, PI-Tli I, PI-Tli II, PI-SceV, megaTALs, SCF, BCL-XL, Foxp3, HoxB4, or SiRT6.

In some embodiments, compositions provided herein may comprise a nanoparticle seed substrate wherein the nanoparticle seed substrate comprises a mutagenized protein.

In some embodiments, compositions provided herein may comprise a payload comprising a nucleic acid encoding a protein.

In some embodiments, compositions provided herein may comprise a nucleic acid encoding a protein, wherein the protein may be selected from a peptide-sequence-guided nuclease, a peptide-sequence-guided transposon, RNA-guided nuclease, an RNA-guided transposon, a DNA-guided nuclease, a DNA-guided transposon, a synthetic PNA/MNA/LNA/modRNA-guided nuclease, a synthetic PNA/MNA/LNA/modRNA-guided transposon, a DNA-repair enhancing protein, Cas9, CasX, CasY, Cpf1, Cas13, MAD7, Rad51, Rad54, transcription activator-like (TAL) effectors (TALEs), TALE nucleases (TALENs), zinc-finger proteins (ZFPs), zinc-finger nucleases (ZFNs), DNA-guided polypeptides, Natronobacterium gregoryi Argonaute (NgAgo), transposons, piggyBac, sleeping beauty, Tc1/mariner, Tol2, PIF/harbinger, hAT, mutator, merlin, transib, helitron, maverick, frog prince, minos, Himar1, meganucleases, I-SceI, I-CeuI, I-CreI, I-DmoI, I-ChuI, I-DirI, I-FImuI, I-FImuII, I-AniI, I-SceIV, I-CsmI, I-PanI, I-PanII, I-PanMI, I-ScelI, I-PpoI, I-SceIII, I-LtrI, I-GpiI, I-GZeI, I-OnuI, I-HjeMI, I-MsoI, I-TevI, I-TevlI, I-TevIII, PI-MleI, PI-MtuI, PI-PspI, PI-Tli I, PI-Tli II, PI-SceV, megaTALs, SCF, BCL-XL, Foxp3, HoxB4, or SiRT6.

In some embodiments, compositions provided herein may further comprise a nanoparticle seed substrate comprising a modified surface-exposed residue, and at least one payload or functional domain coupled to the seed substrate through covalent bonding or complementarity with a PNA, MNA, LNA, RNA, DNA, charged sequence, aptamer sequence, or other polymer with binding affinity for the payload or functional domain, wherein incorporation of the payload or functional domain leads to a stapling conjugation to a modified-surface exposed residue on the seed substrate.

In some embodiments, compositions provided herein may further comprise at least one payload or functional domain coupled to the nanoparticle seed substrate by a protein acting as a binding element.

In some embodiments, compositions provided herein comprising a protein acting as a binding element may comprise a wildtype protein or a chimeric protein.

In some embodiments, compositions provided herein comprising a wildtype protein acting as a binding element may comprise a DNA-binding protein or an RNA-binding protein.

In some embodiments, compositions provided herein comprising a chimeric protein acting as a binding element may comprise a DNA-binding protein or an RNA-binding protein.

In some embodiments, a method for modeling the surface charge of a nanoparticle seed substrate is provided. That method may include generating a three-dimensional model of a nanoparticle seed substrate, wherein the nanoparticle seed substrate comprises a plurality of binding patches, generating a Poisson-Boltzmann electrostatic surface charge plot of the nanoparticle seed substrate to overlay on the three-dimensional model, and identifying one or more surface-exposed residues within a binding patch for modification, wherein the one or more surface-exposed residues are not catalytically active, and wherein the one or more surface-exposed residues are not required for activity of the nanoparticle seed substrate with a binding substrate.

In some embodiments, a method for modeling the surface charge of a nanoparticle seed substrate may include generating a three-dimensional model of a nanoparticle seed substrate, wherein the nanoparticle seed substrate comprises a plurality of binding patches, generating a Poisson-Boltzmann electrostatic surface charge plot of the first nanoparticle seed substrate to overlay on the three-dimensional model, and using the overlay on the three-dimensional model to simulate the addition of a plurality of moieties capable of interacting with the plurality of binding patches to the nanoparticle seed substrate to perform charge surface engineering of the nanoparticle seed substrate.

In some embodiments, the method for modeling the surface charge of a nanoparticle seed substrate may comprise modeling the surface charge of a nanoparticle seed substrate according to a composition of the present disclosure.

In some embodiments, the method for modeling the surface charge of a nanoparticle seed substrate may further include generating a three-dimensional model of a seed substrate, wherein the seed substrate comprises a plurality of binding patches, and wherein the seed substrate is a protein. The method may further include simulating random mutagenesis of the seed substrate in the three-dimensional model, wherein simulating random mutagenesis of the seed substrate facilitates seed substrate surface design.

In other embodiments, a method for predictive modeling of a self-assemblable nanoparticle is provided. That method may include generating a three-dimensional model of a first nanoparticle seed substrate having a zwitterionic three-dimensional surface comprising a plurality of cationic and a plurality of anionic binding patches; generating a Poisson-Boltzmann electrostatic surface charge plot of the first nanoparticle seed substrate to overlay on the three-dimensional model; and using the overlay on the three-dimensional model to charge-tune the first nanoparticle seed substrate by (1) identifying one or more surface-exposed residues within a binding patch for modification, wherein the one or more surface-exposed residues are not catalytically active, and where the one or more surface-exposed residues are not required for activity of the protein with a binding substrate, and (2) using the overlay on the three-dimensional model to simulate the addition of a plurality of moieties capable of interacting with the binding patches to the seed substrate to perform charge surface engineering of the seed substrate.

In some embodiments, a method for predictive modeling according to the present disclosure may further include the first nanoparticle seed substrate having a zwitterionic charge ratio, wherein the zwitterionic charge ratio is defined by the number of the cationic binding patches to the number of anionic binding patches (+/−).

In some embodiments, the method may include modifying the zwitterionic charge ratio, wherein modifying the zwitterionic charge ratio generates a zwitterionic charge-tuned three-dimensional surface.

In some embodiments, at least one of the plurality of moieties may include a modified protein according to a composition of the present disclosure.

In some embodiments, at least one of the plurality of moieties may include a payload.

In some embodiments, the payload may include a biofunctional molecule.

In some embodiments, at least one of the plurality of moieties may include a functional domain.

In some embodiments, the functional domain may include a functional domain according to a composition of the present disclosure.

In some embodiments, the functional domain may be selected from a β-peptide, a γ-peptide, an δ-peptide, an α-helical peptide, a random-coiled peptide, a β-sheet peptide, a σ-strand peptide, a peptidomimetic foldamer, a nucleotidomimetic foldamer, or an abiotic foldamer.

In some embodiments, at least one of the plurality of moieties may include a non-amino acid-based polymer.

In some embodiments, the non-amino acid-based polymer may be selected from a PAMAM dendrimer, a modified PAMAM dendrimer, a functionalized PAMAM dendrimer, a branched polymer, a linearly branched polymer, a dendrimerically branched polymer, a sugar, a glycosaminoglycan (GAG), a proteoglycan, a polysaccharide, a poly(nucleotide), a poly(β-amino ester), a modified amino acid, a modified amino acid mimetic, a peptide sequence containing α-aminoisobutyric acid, a poly(nucleotide) mimetic, a peptide-mimetic, a peptoid-mimetic, a PEG chain, an HPMA chain, a β-peptide, γ-peptide, or δ-peptide, a peptoid, a sigma strand peptoid, a peptidomimetic foldamer, a nucleotidomimetic foldamer, an abiotic foldamer, poly(lactic acid), poly(glycolic acid), poly(lactic-co-glycolic acid), an aliphatic chain, a lipid-derivative, a native lipid, a synthetic lipid, a hybrid polymer, or a multibranched polysaccharide.

In some embodiments, the method may further include using the overlay on the three-dimensional model to simulate addition of a plurality of payloads to the first nanoparticle seed substrate, wherein the payloads may include a plurality of donor DNA molecules.

In some embodiments, the addition of the plurality of donor DNA molecules may enhance surface stability of the nanoparticle seed substrate.

In some embodiments, the addition of the plurality of donor DNA molecules may provide increased efficiency for subsequent gene modulation.

In some embodiments, the subsequent gene modulation may be achieved by insertional mutagenesis, gene ablation, gene correction, transient gene suppression, transient gene expression, multiplexed gene editing, or multimodal combinations thereof.

In some embodiments, the plurality of donor DNA molecules may form a tunable surface for further layering.

In some embodiments, the method may further include a plurality of charged sequences, wherein the plurality of charged sequences supercondense the donor DNA molecules around the nanoparticle seed substrate, wherein the supercondensed donor DNA molecules form a monolayer.

In some embodiments, the method may further include a plurality of anchors, anchor-linkers, or anchor-linker-functional domains are coupled with the monolayer.

In some embodiments, the method may further include using the overlay on the three-dimensional model to pattern a plurality of payloads or a plurality of functional motifs upon the nanoparticle seed substrate surface.

In some embodiments, the method may further include the plurality of payloads or the plurality of functional motifs selected from a multi-threaded predictive ligand, a biologically active polymer sequence, a peptide sequence, a dsDNA cassette, an ssDNA cassette, an mRNA, a dsRNA, a miRNA, a siRNA, a morpholino nucleic acid (MNA), a locked nucleic acid (LNA), peptide nucleic acid (PNA), a biologic, a biologic-drug conjugate, or a polymer-drug conjugate.

In some embodiments, the method may include at least one payload or at least one functional motif that may be a PEGylated payload or functional motif.

In some embodiments, the method may include at least one payload or at least one functional motif that may be an HPMA-modified payload or functional motif.

In some embodiments, the method may further include at least one payload or functional domain coupled to the seed substrate by covalent bonding or complementarity with a peptide nucleic acid (PNA) molecule, a morpholino nucleic acid (MNA) molecule, a locked nucleic acid (LNA) molecule, an RNA molecule, a DNA molecule, a charged sequence, an aptamer sequence, or a polymer with binding affinity for the payload or functional domain, wherein incorporation of the payload or functional domain leads to a stapling conjugation to a modified-surface exposed residue on the first nanoparticle seed substrate.

In some embodiments, the method may further include the payload or functional domain including a PNA-peptide or PNA-functional domain, and wherein the PNA-peptide or PNA-functional domain may be coupled to a modified-surface exposed residue on the seed substrate via stapling conjugation to an end of the donor DNA molecule through base-pair complementarity.

In some embodiments, the method may further include at least one payload coupled to the nanoparticle seed substrate by a wildtype or chimeric DNA-binding or RNA-binding protein acting as a binding element.

In some embodiments, the method may include the wildtype or chimeric DNA-binding protein is selected from Rad51, Rad54, transcription activator-like effector, zinc finger protein, homing endonuclease guide domain, meganuclease guide domain, megaTAL, single-stranded binding protein, TATA-binding protein, helix-loop-helix, helix-turn-helix, leucine zipper, or viral domain.

### BRIEF DESCRIPTION OF THE DRAWINGS

The patent or application file contains at least one drawing executed in color. Copies of this patent or patent application publication with color drawing(s) will be provided by the Office upon request and payment of the necessary fees.

FIGS. 1A-1B depict a three-dimensional simulation model of adenosine deaminase (PDB ID: 5HP3; SEQ ID NO:49) and dCpf1-crRNA-DNA complex (E993A, PDB ID: 5KK5; SEQ ID NO:50) in a PyMol session containing all CPP2.0 cell-penetrating peptides (SEQ ID NOs:52-56). For the displayed peptides, cationic amino acids of constituent peptides (arginines, lysines, and histidines) are colored blue.

FIGS. 2A-2U depict a three-dimensional simulation model of a plurality of ligands, a plurality of anchors, and a plurality of anchors simultaneously loaded onto a nanoparticle seed substrate, whereby the ligands have variable linker domains and static anchor and ligand domains (FIG. 2A: SEQ ID NOs:1, 57-69; FIG. 2C/2D: SEQ ID NO:41 residues 1-168, SEQ ID NO:40 residues 1-165, SEQ ID NOs:70-85, SEQ ID NO:2, SEQ ID NO:40 residues 22-186; FIG. 2E: SEQ ID NOs:81, 1, SEQ ID NO:4 residues 4-167); FIGS. 2F-2S, 2U: SEQ ID NO:41 residues 4-164, SEQ ID NOs:81, 66.

FIG. 3 depicts three three-dimensional simulation models: a Rad51-DNA complex (left), a Cpf1 RNP (middle), 31nt DNA (top right), and an exemplary anchor-linker-ligand formation (bottom right, anchor in blue, linker in green, cleavable domain in orange, ligand in pink).

FIGS. 4A-4H depict Cpf1 selective mutagenesis, whereby non-surface-exposed and/or catalytically important inwardly-facing residues are maintained in their wildtype state. Exposed residues and non-critical residues for activity are highlighted in green. Overlay with Poisson-Boltzmann plots allows for determination of key surface mutagenesis sites, including ranges of amino acids (FIG. 4H, amino acid residues 175 (P) through 268 (D)) that represent charged non-activity-critical pockets.

FIG. 5 depicts a three-dimensional simulation model of a Cpf1-gRNA complex (left) and an anchor-linker-ligand (right). The anchor is a cationic H2AX-derived anchor [119-143] anchor (blue), a linker (green), a cleavable domain (orange), and a ligand (pink).

FIGS. 6A-6B depict three-dimensional simulation models of Rad51 bound to an ssDNA (left model in FIGS. 6A and 6B) and Cpf1 with Poisson-Boltzmann electrostatic surface potential plots (overlay) in complex with sgRNA (right model in FIGS. 6A and 6B).

FIG. 7 is a chart depicting intensity profiles of Cas9 RNPs in the 1-20 nm range prior to nanoparticle formation in buffer conditions defined in Table 1 (providing buffer and pH conditions that may be utilized for achieving efficient electrostatic nanoparticle condensation).

FIG. 8 is a chart depicting particle sizes of CRISPR RNP following a number of electrostatic self-assembling nanoparticle formulations. The color coding represents a heatmap view of particle sizes. The color-coded boxes represent PLR10 titrations (left) and PLK10-PEG22 titrations (right) performed via Andrew robot nanoparticle synthesis prior to particle measurement with a Wyatt Mobius Zeta Potential and DLS detector.

FIG. 9 depicts a series of three-dimensional simulation models examining size considerations that may contribute to why poly(L-arginine) (n=10) and PLK10-PEG22 consistently formed CRISPR RNP nanoparticles in the 20-59 nm range. Measurement line (yellow) is used to determine hydrodynamic size in the PyMol software. Here, Cas9 is determined to be ˜12 nm in diameter in the axis measured. PLR10 is shown approximately to scale, at ˜3.5 nm. Positive charge patches are depicted in blue and negative charge patches are depicted in red. PLR10 is predicted to fit into the negatively charged patches and assist in breaking apart the zwitterionic aggregates. The green portions with blue and red side chains represent the gRNA exposed residues.

FIGS. 10A-C depict depicts three sequence-alignment based simulations of the sequence KKRTSATVGPKAPSGGKKATQASQEYFKFLGGGGSGGGGSFKFLMIASQFLSALTLVLLI KESGA (representative anchor-linker-ligand; SEQ ID NO:1), whereby Phyre2 was used to model the sequence. The N-terminus (red) may have 6 cationic residues capable of intercalating with anionic components of a preceding layer or associated component. The C-terminus (blue) represents an E-selectin ligand, and as with any ligand of less than 20 amino acids in length, the structure is predicted to be a random coil with an “induced fit” upon binding to the appropriate receptor or protein/other target.

FIG. 11 depicts a comparison of predicted anchor-linker-ligand structures for KKRTSATVGPKAPSGGKKATQASQEYFKFLGGGGSGGGGSMIASQFLSALTLVLLIKESG A (SEQ ID NO:2), to scale, with Cpf1 RNP (middle) and Rad51-ssDNA complex (left) shown.

FIGS. 12A-12B depict three-dimensional simulation models of Rad51 (left), Cpf1 RNP (middle), and a variety of relevant anchor, linker and/or ligand sequences.

FIG. 13 depicts a three-dimensional simulation model of a ligand simulation, which demonstrates a multi-domain peptide sequence whereby the blue electrostatic domain (which may have various extensions of histidines, arginines and/or lysines, NLSs, and the like on its N-terminus or throughout the H2AX-derived sequence), cleavable domain (FKFL; SEQ ID NO:3), linker domain (GGGGSGGGGS; SEQ ID NO:4), and E-selectin ligand domain (MIASQFLSALTLVLLIKESGA; SEQ ID NO:5).

FIGS. 14A-14H depict three-dimensional simulation models of linker alignments (grey) alongside various identical anchor and ligand motifs.

FIG. 15 depicts a three-dimensional simulation model of ligand domain (E-selectin ligand, pink), cleavable domains (FKFL (SEQ ID NO:3), yellow), linker domain (GGGGSGGGGS (SEQ ID NO:4), GAPGAPGAP (SEQ ID NO:6), and the like, orange), and anchor domain (histone H2AX-derived domain with optional S139 phosphorylation, blue) with partially transparent Poisson-Boltzmann plot overlaid.

FIG. 16 depicts a three-dimensional simulation model of an E-selectin ligand, a cleavable linker, and an anchor with partially transparent Poisson-Boltzmann plot overlaid. Large cationic surface potential areas (blue) will preferentially dissociate from their self-folding behavior following interaction with an anionic surface potential, nucleic acid, amino acid, and the like.

FIG. 17 depicts a three-dimensional simulation model of an H2AX-derived (119-143) anchor domain with an optional extensible electrostatic domain (1-18) that may be modified with additional histone domain fragments, KKR motifs, NLS, cationic charges, anionic charges, hydrophobic AA, hydrophilic AA, hydrogen bonding AA, and the like, including Phosphorylated and otherwise post-translationally modified variants thereof and H4-derived histone variants thereof (highlighted), as well as any other factors or fragments thereof that may promote DNA repair or cell-cycle dependent processes. A partially transparent Poisson-Boltzmann plot is overlaid. The blue region represents a region where additional histone fragments, NLS, or other appropriate sequences may be incorporated. For example, KRR or the like may be added to the N-terminus to act as an additional NLS.

FIG. 18 depicts a three-dimensional simulation model of a cleavable linker domain (highlighted) with partially transparent Poisson-Boltzmann plot overlaid.

FIG. 19 depicts a three-dimensional simulation model of an E-selectin ligand domain (highlighted) with partially transparent Poisson-Boltzmann plot overlaid.

FIG. 20 is an exemplary depiction of a predicted contact and distance matrices. Darker boxes indicate more interaction. Here, linker domains, for example, are shown to have minimal self-interaction due to being proximal to each other, while distal ends have more interaction with each other. Electrostatics play a role in defining contact map matrices.

FIGS. 21A-21B are three dimensional models depicting PDB ID: 1G1T (E-selectin) bound to sialyl Lewis X (SIA) (FIG. 21A) and PDB ID: 1G1S (L-selectin) bound to SIA (FIG. 21B). FIG. 21C is a sequence alignment of 1G1T (E-selectin) and 1G1S (L-selectin) showing that binding sites to sialyl Lewis X remain identical in E-selectin and L-selectin. FIG. 21D is a three-dimensional model overlay depicting both E-selectin and L-selectin bound to sialyl Lewis X.

FIG. 22 depicts a three-dimensional model of a quatrimer of recombinant human SCF with key binding residues highlighted. N-terminal modification with anchor-linkers maintains the outwardly facing binding domain.

FIGS. 23A-23B depict the binding domains of Plasmodium falciparum derived reticulocyte binding protein 5 (red=binding domains, orange=rest of protein), bound to CD147 (teal) (PDB ID: 4UOQ).

FIGS. 24A-24C show CD47 (blue) and SIRPa (salmon). Strong (red) and weak (magenta) binding domains are also shown.

FIGS. 25A-25C show CD4 (blue) and HIV gp120 (yellow; SEQ ID NO:86) along with key binding domains of gp120 for binding to CD4 (in red).

FIG. 26 is a graph depicting florescence intensity over time to examine ribonucleoprotein particle (RNP) crosslinking with double stranded DNA (dsDNA) and without dsDNA using a SYBR inclusion assay.

FIG. 27 is a color-coded table depicting results of a SYBR inclusion assay.

FIG. 28 is a color-coded table depicting results of a SYBR inclusion assay.

FIG. 29 is a color-coded table depicting results of a SYBR inclusion assay.

FIG. 30 is a color-coded table depicting results of a SYBR inclusion assay.

FIGS. 31A-31B depict three-dimensional models of Rad51-Cas9 modified fusions. Cas9 RNP. Red=regions shown to abolish Cas9 activity in the literature. Orange=regions shown to reduce activity. Yellow=regions shown to cause nickase activity. Green=sites for charge manipulation of the surface as determined by APBS electrostatic density plots.

FIG. 32 is a three-dimensional model depicting IL2 (SEQ ID NO:87)-IL2RA (SEQ ID NO:88) binding (PDB ID: 1Z92) as determined by contact mapping via PDBePISA.

FIG. 33 is a three-dimensional model depicting IL2-IL2RA binding where red sequences of a known sequence may be mutated into variants that do not form a repulsive effect with the corresponding red sequences shown in the IL2RA receptor or another wildtype receptor with a known structure.

FIG. 34 is a series of tables detailing PISA interface and interfacing structures in Example 8.

FIG. 35 depicts a three-dimensional model showing the crystal structure of human CD3-e/d dimer in complex with a UCHT1 single-chain antibody fragment (PDB ID: 1XIW).

FIG. 36 depicts three-dimensional modeling of CD28 and an antibody which can be compressed to 7 amino acids to generate affinity as predicted by AG values between the binding residues on CD28. This antibody can be compressed to 7 amino acids to generate affinity as predicted by AG values between the binding residues on CD28.

### DETAILED DESCRIPTION

Provided herein are certain compositions and methods for nanoparticle seed substrates for self-assimilable nanoparticles and methods for optimized design of the same.

Using current nanoparticle design approaches known in the art, if particle formation begins with a 100 nm Cas9 ribonucleoprotein particle (RNP) aggregate, 100 nm particles will form unless additional modifications are performed by thermodynamically stabilizing the Cas9 RNP monomers/dimers/trimers/etc. in such a way that limits protein-protein interactions. One example of protein interactions is apparent in the case of Cas9 and Cpf1 RNPs, which are highly zwitterionic. In certain embodiments disclosed herein, modification of key surface-exposed residues on a nanoparticle seed substrate for subsequent charge engineering of the surface via Poisson-Boltzmann plots provides a novel technique for increasing colloidal stability of ribonucleoprotein (RNP) or protein monomers and preventing aggregation (e.g. an electrostatic stacking effect). In certain embodiments, the modifications do not impair protein function because they are not tied to key catalytic residues and domains and their subsequent functions. In certain embodiments, modifications, such as eliminating positive charges from Cas9 or Cpf1 RNP, may enhance gene editing activity and reduce off-target mutagenesis (possibly due to less Cas9 affinity to anionic nucleic acids when the Cas9 surface is more negatively or neutrally charged). In any event, applicant has generated data demonstrating that “matching electrostatic pocket” polymers must be a similar size to the to-be-charge-switched anionic pocket(s), in order to cause Cas9 RNP dissociation. It is predicted through various structural modeling approaches how to charge modify the RNP surface of a number of proteins, and how to predictively assemble anchor, linker and/or ligand/functional domains upon the surface of a surface.

In certain embodiments, molecular dynamics simulations through neural network-based approaches are utilized along with existing X-ray crystallography and NMR data of structures in RCSB Protein Data Bank (PDB). In certain embodiments, various anchor, linker, and ligand domains are optimized for certain delivery application based on visualization and sequence alignment of key surface-binding residues (e.g. charged anchors, covalent anchors and the like) upon cell-specific, tissue-specific, organ-specific heteromultivalent, or homomultivalent display of peptide ligands. Various known and de novo sequences are utilized to create binding affinity for a given receptor profile of a target cell/tissue/organ population. A database of cell-penetrating peptides and associated PDB files is downloaded from CPPSite2.0 and is used as a basis for rational engineering of histone-derived, mRNA-optimized, DNA-optimized, ssODN-optimized, RaptorX-simulated de novo ligands from native protein fragments and structurally-useful domains (e.g. for a spacer, for ssODN intercalation, for dsODN intercalation, for electrostatic pocket neutralization, for site-specific conjugation to a protein or nucleic acid substrate, for a linker, for a cleavable domain, and the like). Many sequences provide as many as 6 unique domains optimized for cell-specific targeting, compartment-specific release, and specific kinds of payloads (e.g. mRNA vs. Cas9 RNP-DNA vs. Cas9 RNP vs. DNA).

In some aspects of the present disclosure, biomimetic self-assemblable nanoparticles and methods for designing the same are provided. In some embodiments, native-protein-derived ligands may be employed in order to furnish cell-cell and cell-ECM interactions between a self-assemblable nanoparticle and a target by creating a “biomimetic” coating or covalent/non-covalent conjugation (e.g., immunomimetic/secretome-mimetic/ECM-mimetic/cell-mimetic ligand, or combination of ligands), which may by themselves be multiple ligands comprising small domains of a single larger protein fragment, or multiple ligands comprising small domains of multiple protein fragments. Fusion proteins of a biomimetic ligand may also be utilized to target chimeric proteins or RNPs without further modification. However, direct modification to a protein surface-either covalently or non-covalently—may be particularly suitable for delivery of nucleic acid-bearing and nucleic-acid-mimetic-bearing cargoes (e.g., RNPs, DNA-RNPs, DNA, RNA PNA, MNA, LNA, and the like).

In some aspects of the present disclosure, self-assemblable nanoparticles for use with various barcoding techniques and methods for designing the same are provided. In some embodiments, a DNA, RNA, PNA, MNA, LNA or other barcoding molecule (e.g., multi-fluorescent barcodes) may be presented within a gRNA or expression RNA/DNA, or upon the surface of a protein or RNP of the disclosure to allow for ultra-high-throughput screening via enhanced selection and parallel study of nanoparticle, gene editing, or gene expression cassettes. In some embodiments, nanoparticle formulations are individually barcoded.

In some aspects of the present disclosure, biofunctional species may be incorporated into self-assemblable nanoparticles of the present disclosure. In certain embodiments, a number of biofunctional species may be simultaneously or individually patterned upon the surface of a biomolecule (e.g., mRNA, DNA, nanoparticles, virus-like particles, Cpf1 RNP, Cas9 RNP, DNA-bound forms thereof, and the like). One limitation in the prior art is that recombinant protein engineering may be capable of providing a good species, however, delivery remains an important missing piece. By tailoring the electrostatic and/or covalent surface of a modified protein or performing recombinant modifications of a ligand in such a way that stabilizes individual protein molecules in a non-aggregated way (Poisson-Boltzmann surface engineering electrostatic displacement with targeting ligands, with associated LGDL_chimeric proteins, e.g. LGDL_Cas9, modCpf1, modCas9_E-selectin, modCas9_CD4, modCas9_cKit, modCas9_ligand, modCas9-anchor-ligand, modCas9-H2AX, modCpf1 Rad54-H2AX_ligand, modCas9-AAsequence, and the like), some aspects of the present disclosure allow for use of electrostatic, covalent, and/or displacement-sensor-based (e.g. aptamers/RNA/DNA/PNA/MNA/LNA and the like) polymers, including nucleic acids and peptides, to control the spatial assembly of cell-specific or cell-penetrating delivery systems.

In some aspects of the present disclosure, predictive modeling of self-assemblable nanoparticles and their components are provided. In certain embodiments, molecular dynamics and simulation tools may be used to predictively assemble optimal anchor, linker, and/or ligand/functional-species upon the surface of, or as part of nanoparticle-based assemblies, bearing a variety of nucleic acid, protein, charged, neutral, or otherwise covalently-modified molecules as electrostatic, PNA/RNA/DNA/LNA/MNA-based affinity, phase-based, and other multilayering and/or monolayering strategies upon substrates that necessitate effective gene and/or protein and/or molecular delivery to provide therapeutic efficacy. These techniques are broadly extensible to techniques for achieving ligand-targeted and/or cell-penetrating behaviors with a variety of bioresponsive, predictable, and cell-tailored efficacies for delivery of gene editing, gene expression, gene suppression, gene insertion, gene ablation, gene correction, transient expression, transient suppression, drug-polymer conjugates, chimeric biologics, and the like. In certain aspects, the present disclosure provides careful controlling of surface chemistries and presentations of various key functional domains for nanoparticle-based assembly and/or self-assembly upon a near-universal set of substrates (e.g., protein, metallic biomaterial or ceramic and/or ceramic-metal biocomposite, water-phase material, oil-phase, electrostatic-phase material, polymer, nucleic acid, peptide, diagnostic nanoparticles, theranostic nanoparticles, and the like).

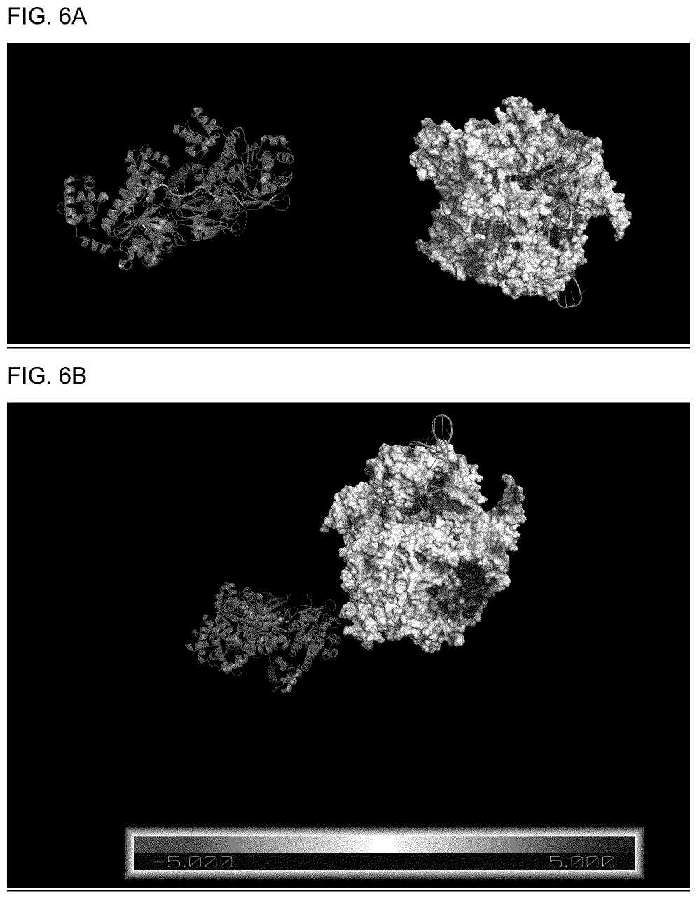

In some embodiments, mutation of residues within cationic sites (see, e.g., FIGS. 6A-6B, cationic sites in blue) to cysteine or another bioconjugatable amino acid substitution may result in an anchor for peptides; polymers; 3′ or 5′ linkage of dsDNA, ssDNA, tetrisDNA, mRNA, PNA, LNA, MNA; or any other suitable linkage. Linkage may be covalent or through a non-covalent complementary annealing bridge that may comprise DNA, RNA, PNA, LNA, MNA, or the like. These techniques may also be applied to anionic sites (e.g., FIGS. 6A-6B, anionic sites in red) on a zwitterionic surface, when a cationic moiety may be covalently linked to the formerly anionic surface amino acids of the wildtype protein which may be used to link polymers, polypeptides, ligands, or other specimens. Embodiments according to the present disclosure may use Poisson-Boltzmann electrostatic surface potential plots overlaid on three-dimensional models in some methods disclosed herein. See https://apbs-pdb2pqr.readthedocs.io/en/latest/releases/201601-APBS-1.4.2.1.html (discussing Poisson-Boltzmann electrostatic surface potential plots).

Following charge homogenization (either through polymer (+, − or 0), peptide (+, − or 0) or nucleic acid (−)/nucleic acid-like (− or 0) sequences), it is then possible to selectively electrostatically assemble either a single-step nanoparticle or multi-step nanoparticles upon the exposed charged surface. Exemplary nanoparticle components and ligands may be found in the following patent applications, which are hereby incorporated by reference in their entirety: U.S. patent application Ser. No. 15/842,820 (issued as U.S. Pat. No. 10,975,388), Ser. No. 15/842,829 (published as U.S. Publ. No. 2018/0179553), Ser. No. 16/387,507 (published as U.S. Publ. No. 2020/0208177), and Ser. No. 16/393,886 (published as U.S. Publ. No. 2020/0149070); and U.S. Provisional Appl. Nos. 62/434,344, 62/443,522, 62/443,567, 62/517,346, 62/659,627, 62/685,243, 62/736,400, 62/661,992, and 62/685,240.

The nanoparticle formation and predictive modeling methods of the present disclosure are not limited to electrostatic nanoparticle assembly, but may also rely upon, for example, complementary base pairs between a PNA, RNA, DNA, DNA origami-like structure, RNA origami-like structure, and/or a peptide, antibody and the like (active targeting fragment, non-limited to peptides and antibodies), whereby the complementary base pairs assemble upon a covalently-modified Cas9 surface (“charge-homogenized surface”) with one or more covalent attachment sites through selective mutagenesis of the Cas9 (or other nuclease/protein) surface, which are subsequently bound to the 3′ or 5′ end of the affinity-generating complementary base pair PNA, RNA, DNA, LNA, MNA, or the like.

In certain embodiments, Rad51 or Rad54 fusion proteins to any nuclease may provide a template for ssDNA binding prior to or following introduction of sgRNA for formation of a DNA-Nuclease-[sgRNA] fusion DNA-ribonucleoprotein (in the case of Cas9 and RNA-guided nucleases) or fusion DNA-protein (in the case of TALEN, meganuclease, and other protein-guided nucleases). The same charge manipulation strategies may be utilized for providing additional sites for donor oligonucleotide (ssDNA/dsDNA) linkage from the 3′ or 5′ end, through techniques according to the present disclosure for providing multiple copies of ssDNA or dsDNA upon the protein surface. A skilled artisan would recognize that the state of the art does not address the “zwitterionic aggregation” problem faced with Cas9 RNPs and related RNP payloads, which self-aggregate due to their heterogenous surface charges. Methods and compositions according to the present disclosure address these challenges by providing for mutation of key charged residues in key binding patches, as determined by Poisson-Boltzmann plots, allowing for charge homogenization of the surface potential. Thus, present disclosure overcomes limitations in the prior art by generating optimized biomolecular scaffolds for electrostatic, covalent, and/or annealing-mediated nanoparticle layer-by-layer formation, self-assemblable nanoparticle components including seed substrates, and methods for predictive modeling of the same.

According to certain embodiments, it may be possible to pattern “multithreaded” predictive ligands and/or cell-penetrating peptides and/or dsDNA/ssDNA cassettes, and the like, upon a protein or RNP surface in such a way that facilitates predictable assembly, limits canonical Cas9 RNP and Cpf1 RNP protein aggregation (due to zwitterionicity), and provides a homogenous surface potential (or bioconjugated domain phase interactions, e.g., lipids) for subsequent cell targeting.

In certain embodiments, a Cas9 RNP with a spatial assembly of numerous functional peptides allows for exemplary protein-binding prediction of peptides, polymers, or any structure that can be inputted into PDB renderings with its associated physicochemical properties. These initial interactions-whether they are electrostatic, hydrophobic, hydrophilic, or hydrogen bonding mediated-allow for either transient stabilization of a phase-bound polymer to the appropriate-phase binding cleft, or alternatively serve for catalyzing additional site-specific crosslinking or covalent bonding approaches at the desired sites. The approach utilizes Poisson-Boltzmann electrostatic surface charge plots of the desired protein, and eliminates protein sequences that are catalytically active or required for activity of the protein with its subsequent binding substrate as possible sites of mutagenesis (“deselection criteria”). The remaining surface-exposed amino acids (“selection criteria”) may be selectively mutated, with the remainder of the binding cleft remaining similar or slightly modified (or neutralized) upon an appropriate polymer/nucleic acid 3′ or 5′ end/PNA/peptide/poly(β-amino ester)/p(asp)[DET], and the like, with the option for a linker domain (e.g., flexible, semi-rigid or stiff; cleavable or not cleavable, and the like) and/or ligand domain, endosomolytic domain, or fluorescent reporter domain.

Embodiments according to the present disclosure may be coupled with genome-wide peptide sequence alignments of desired binding patches, with comparison to the desired organism's (e.g., a human, mouse, canine, etc.) native sequences (for minimizing immunogenicity and maximizing on-target effect), whereby a sequence for binding may be selected based on its “scoring matrix” of hydrophobic-hydrophobic interactions, hydrophilic-hydrophilic interactions, hydrogen bonding interactions, electrostatic interactions, and the like as relate to predicted binding interactions. While hydrophobic-hydrophobic, hydrophilic-hydrophilic and hydrogen bonding phase interactions will have a positive score (+n) for sequence similarity (e.g., leucine, isoleucine and valine for hydrophobic-hydrophobic affinity), electrostatic interactions will be scored -m for each similar interaction (e.g., arginine-lysine, lysine-histidine, glutamic acid-glutamic acid, and the like) and +m for each opposing interaction (e.g., arginine-glutamic acid, glutamic acid-histidine, lysine-aspartic acid, and the like). According to embodiments disclosed herein, sequences of any cationic or anionic, as well as neutral or hydrophobic peptide, may be substituted in whole or in part by synthetic or natural polymers/aliphatic chains/PNAs/etc. with similar physicochemical properties (e.g., charge, hydrophilicity, hydrophobicity, or lack of phase-association behavior other than covalent modification prior to subsequent assembly upon the now-conjugated covalently bound motif, and the like).

In some aspects of the present disclosure, modifications to adenosine deaminases, dCas9, dCpf1 and the like may also be performed to maintain activity of one or both proteins of the RNP or guided nuclease, while serving similar purpose for engineering electrostatic and/or covalent and/or linked attachment sites/protein surfaces. A skilled artisan would understand that the embodiments herein need not include co-delivery embodiments, as the concept of electrostatic modulation of zwitterionic proteins for nanoparticle assembly and limitation of aggregation is a novel concept. A skilled artisan would also understand that any catalytically active site, or catalytically inactivating site, or otherwise functionally-useful site (e.g., D10A, dCas9-fusions, epigenetic modulation and the like) may be mutated in conjunction with or independently of surface mutagenesis, as the electrostatic principles for zwitterionic protein assembly may be applied to even native proteins. A principal advantage of surface residue engineering is to limit aggregation prior to nanoparticle assembly, as the seed substrate's size is critical for layer-by-layer or monolayer formation and subsequent activity of approaches for targeted nanomedicine, ligand-protein conjugate delivery, and the like.

In some aspects, the present disclosure provides diagnostically-responsive functional domains for incorporation into self-assemblable nanoparticles. In this aspect, it is possible to use transcriptomics (e.g., RNA-Seq) and/or proteomics data to determine surface markers of targets (e.g., a target tissue). These markers can be subclassified to identify which of those markers have PDB renderings available, and which of those PDB renderings for transmembrane receptor surface domains have known binding partners or homologues that can be simulated in their binding to the protein. Next, using three-dimensional modeling approaches provided herein, thermodynamic modeling can be performed to determine AG values between individual residues. Modeling approaches provided herein may be used to create a functional domain that can be used to target the desired tissue type. In certain embodiments, contact and distance mapping may be used to modulate AG values. Self-assemblable nanoparticles according to the present disclosure may be used to deliver a gene, a small molecule, or a protein complex by incorporating functional domains as detailed herein.

From the foregoing, it will be appreciated that specific embodiments of the invention have been described herein for purposes of illustration, but that various modifications may be made without deviating from the scope of the invention. Accordingly, the invention is not limited except as by the appended claims.

### Examples

The following examples are illustrative and not intended to limit the scope or content of the present disclosure in any way.

### Example 1—Predictive Modeling of Self-Assemblable Nanoparticles and Components: Anchor/Linker/Ligand Interaction Design

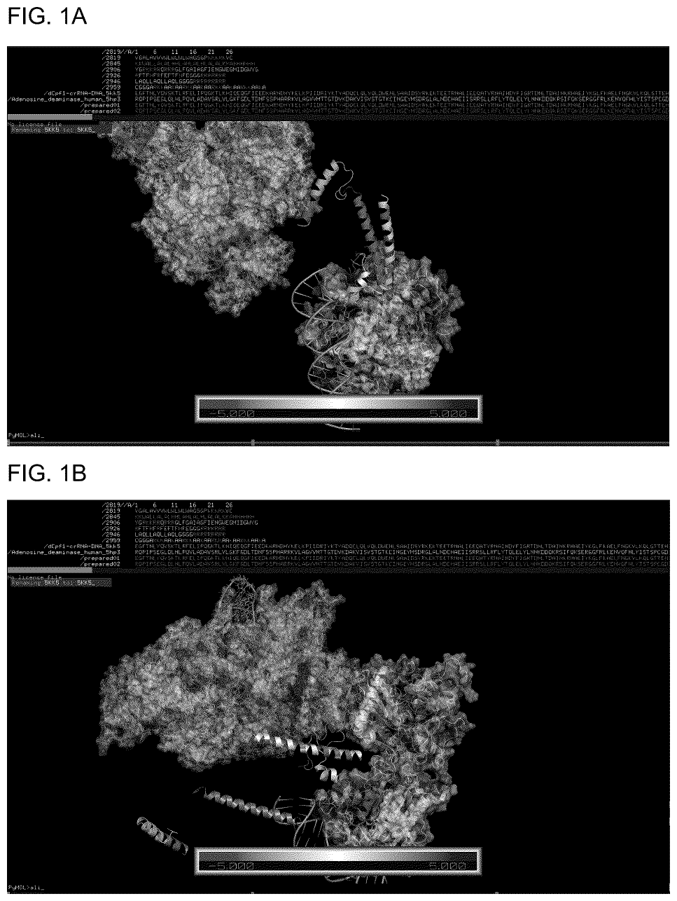

Predicative modeling techniques according to the present disclosure may be used to visualize self-assemblable nanoparticle components including seed substrates, anchors, linkers, ligands, or any other appropriate binding partner. FIGS. 1A-B provide a three-dimensional model depicting adenosine deaminase (PDB ID: 5HP3) and dCpf1-crRNA-DNA complex (E993A, PDB ID: 5KK5) in a PyMol session containing all CPP2.0 cell-penetrating peptides. For the displayed peptides, cationic amino acids of constituent peptides (arginines, lysines and histidines) are colored marine (blue). Ratio of peptide to protein/RNP can be predicted so as to neutralize anionic or cationic surface pockets of a native or chimeric protein/RNP. The length and shape of each peptide can influence how many molecules are required to neutralize a given electrostatic surface pocket. For example, while alpha helical cationic peptides are likely to intercalate into pockets and represent an optimized “packing function” on the surface due to their bulky shape, random-coiled sequences (such as KKKRK (SEQ ID NO:9), KKAHHHHHH (SEQ ID NO:10), RRRRRRR (SEQ ID NO:11), RRRRRRRRR (SEQ ID NO:12), and the like) are more likely to act as “loose strings” that exhibit more predictable stochiometric charge neutralizing behaviors. Addition of linkers and ligands can create similar spacing patterns, where the volume of the peptides is modeled versus the charged pocket through sequence alignment approaches and manual fitting of peptides, and/or electrostatic/ligand-binding affinity simulation upon the waterboxed protein surface.

Fusion proteins and similar fusion proteins thereof (as well as non-protein conjugates, e.g., other substrates, nucleic acids, PNAs, and the like), not necessarily of the same function as a given fusion protein in the state of the art, may also be used with these novel self-assembling layering approaches. An additional aspect to consider is a gBaseEditor, whereby a Cas9 or Cpf1 (or other nuclease/protein) substrate is modified to possess affinity for a gRNA (or a sequence for TAL/ZFP/meganuclease-mediated binding and the like). The chimeric fusion protein or ribonucleoprotein (or DNA/PNA/LNA/MNA-guided protein) may be modified with adenosine deaminase and the like, in the absence of typical structures necessary for nuclease catalysis, whereby tailoring the length of a “linker” (amino acid, polymer or aliphatic linker) between the RNA/DNA-scaffold-binding-protein (e.g. Cas9/Cpf1, NgAgo, and the like) and its no-longer-necessary functional domains for performing a site-specific cleavage. Therefore, the base editor presentation need not rely on more than a small percentage <20% of total structure for anchoring to an appropriate gRNA or gDNA substrate, whereby the catalytically active enzyme may present site-specifically to sets of predicted base pairs. Additionally, a protein substrate is not necessary. A PNA-guided, RNA-guided, DNA-guided, LNA-guided, MNA-guided, or other affinity-guided (e.g., protein affinity, compartment-specific affinity, peptide affinity) base editor, transcriptional activator, transcriptional repressor, or other functional enzyme/protein may be delivered to have affinity for specific base pairs or proteins in this way.

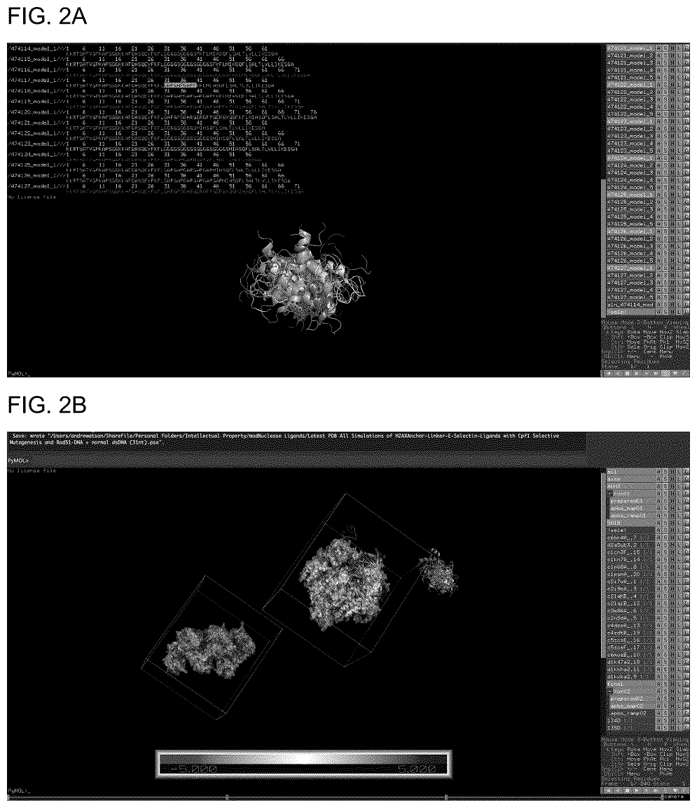

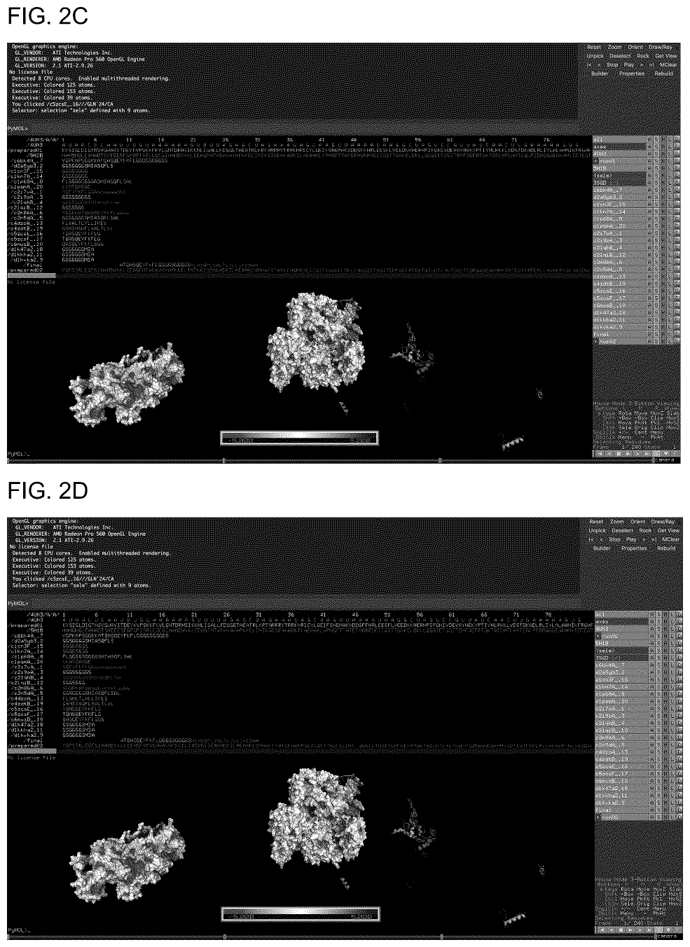

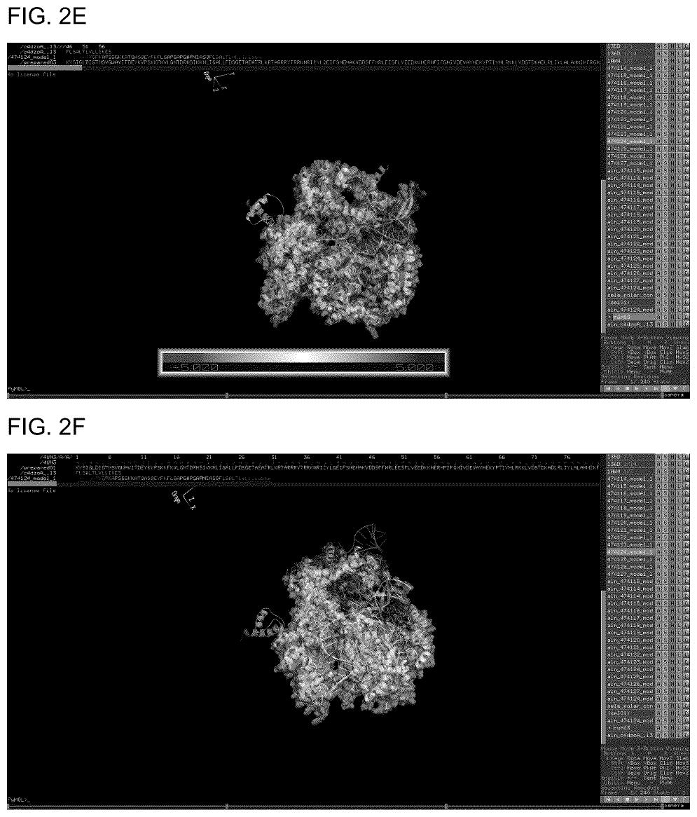

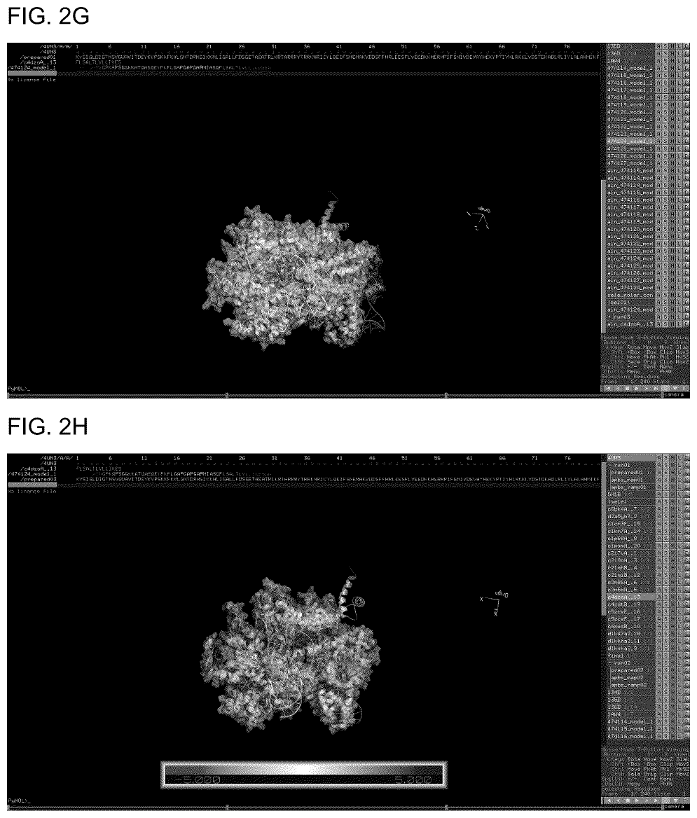

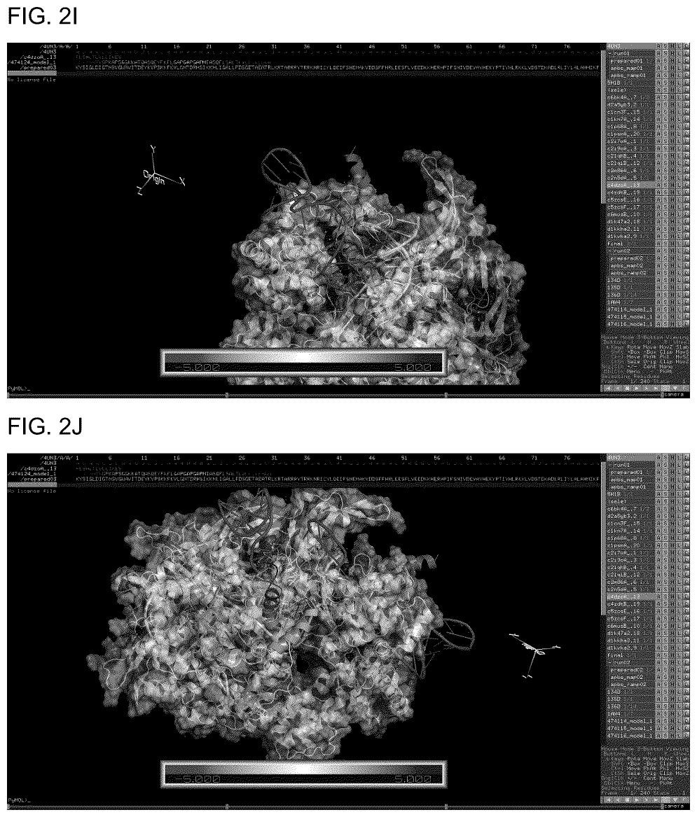

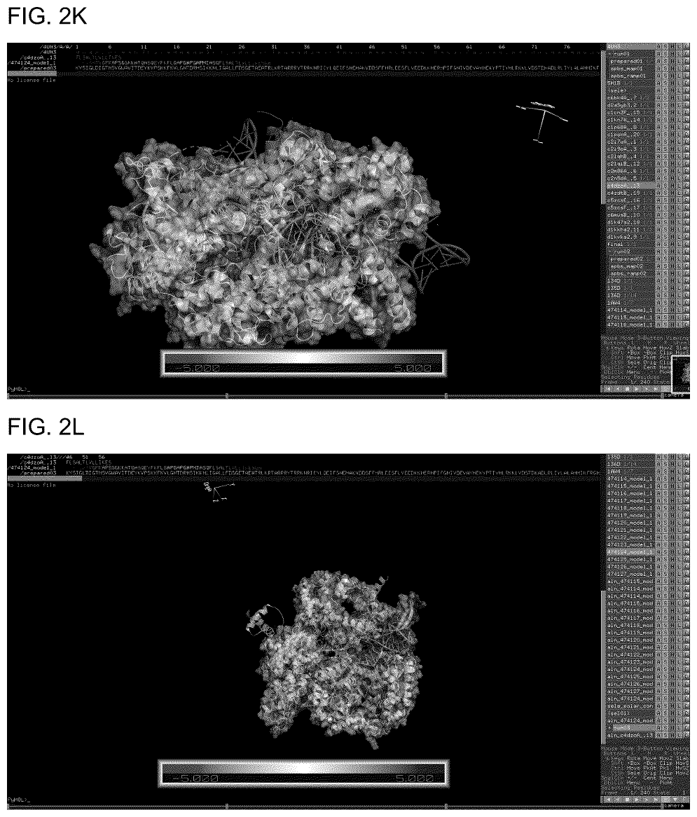

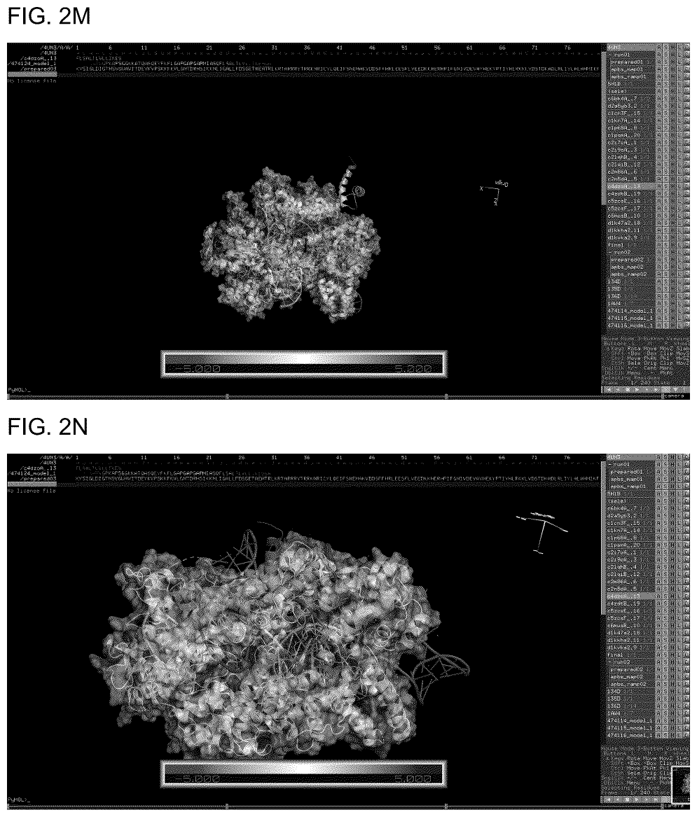

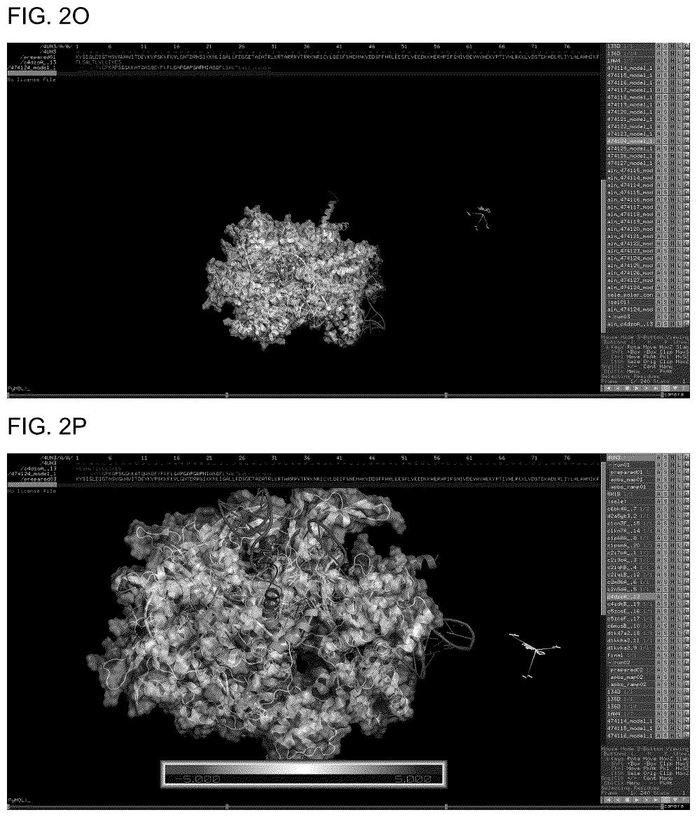

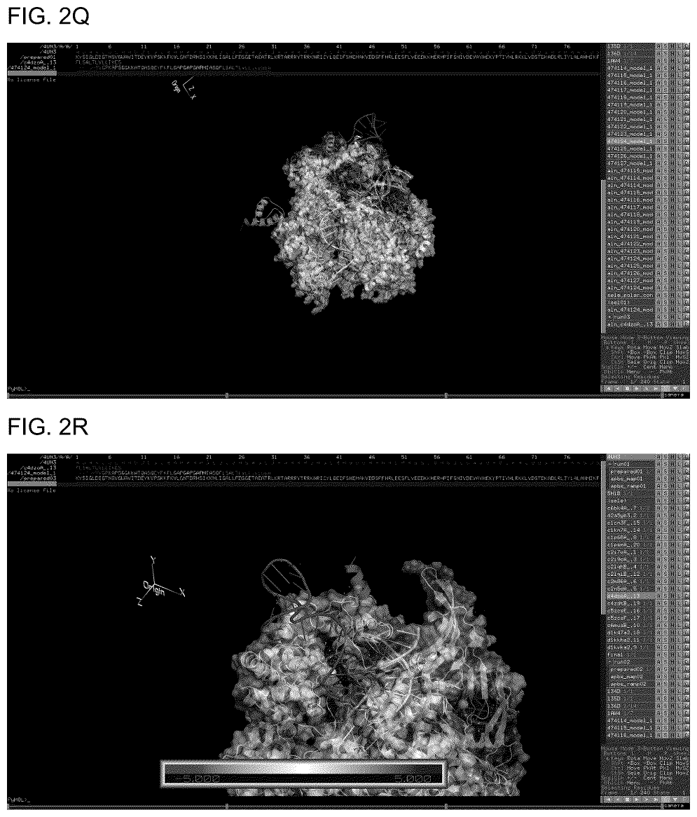

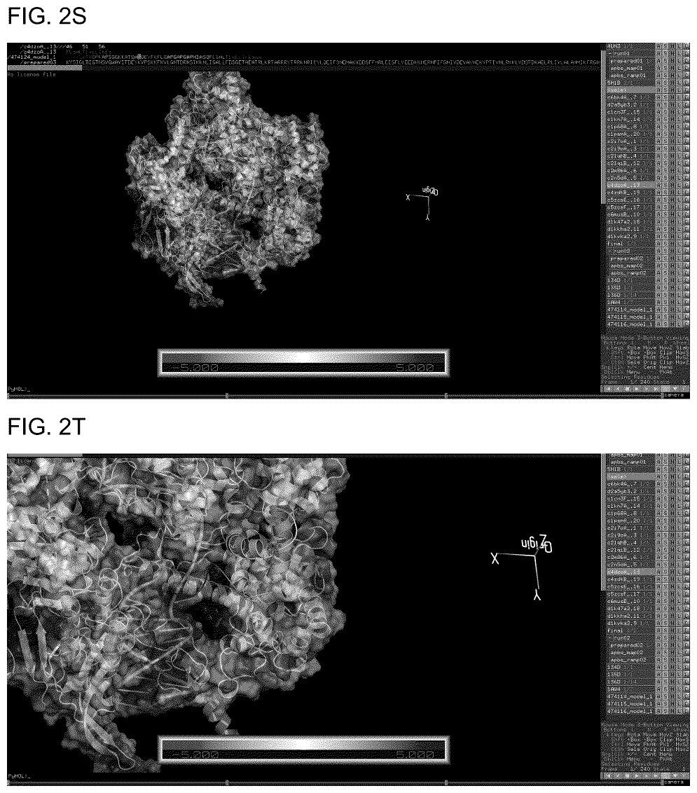

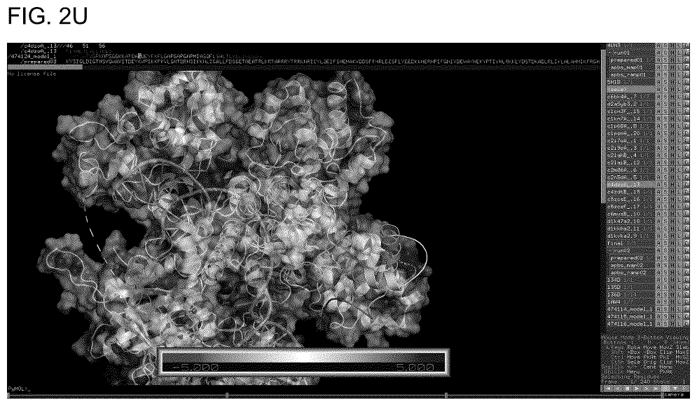

FIGS. 2A-U depict dozens of ligands, anchors, and linkers simultaneously loaded, whereby the ligands have variable linker domains and static anchor and ligand domains. In FIGS. 2B-D the following are modeled: Rad51-ssDNA (left), Cpf1-RNP (middle) and H2AX-linker-E-selectin ligand permutations (right). Any one of the loaded peptide species may be covalently or non-covalently linked to an underlying substrate (of any kind, not necessarily a nanoparticle or RNP or protein of the disclosure), following predictive binding or mutated residue-mediated binding as described elsewhere. The anchor and ligand domains may also be varied in such models. These modeling techniques allow for approximating self-folding behavior and likelihood of anchor interaction with the desired protein, surface, or payload substrate.

These ligands may be multivalently assembled and simulated for their binding to an electrostatic surface (or another binding surface, as described elsewhere), which can serve as its own delivery system in serum-free media, or serve as covalent conjugation (e.g., site-specific S—S bond) facilitation on a cysteine-modified or otherwise modified RNP surface. Additionally, electrostatically bound initial anchor-linker-ligands have been demonstrated to be effectively stabilized by subsequent inclusion of anionic polymers, including nucleic acids and PLE/PDE, in our studies. Electrostatic anchors, on their own or with covalent/other anchors assembled elsewhere upon the RNP as described herein, may also be particularly well suited for shielding gRNA during endocytosis. The anionic layering step serves to “electrostatically crosslink” chains of cationic residues between anchors and can also serve to modulate protease and nuclease resistance due to optional inclusion of D-isomer amino acids and manipulation of endosomal sorting pathways (non-lysosomal pathways) that are preferentially triggered by smaller particles with receptor-mediated endocytosis.

For conjugation, an amine-reactive or amide-reactive chemistry may also be used to connect an anchor, linker, active species, passive species, stabilizing species, ligand or combination thereof (not necessarily an electrostatic anchor) to a protein, and in either way the initial affinity of the anchor to its protein substrate will guide biased and site-specific predictive covalent conjugation in the event that such a self-assembly is desired. Predictive patterning of non-critical surface domains during selective mutagenesis allows for modulation of crosslinking sites; in other examples electrostatic and/or phase-based guides (polymer sequences as described elsewhere) facilitate local phase or charge-mediated crosslinking locally to appropriately phased bioconjugatable sites. The linkages and species involved may include maleimide linkages, cysteine residues, amine-reactive chemistry, amide-reactive chemistry, phosphoramidite chemistry, 3′ and 5′ chemistry, N-terminal chemistry, C-terminal chemistry, COO-reactive chemistry, NH2-reactive chemistry, NH3-reactive chemistry, S04-reactive chemistry, lipid-reactive chemistry, side-chain reactive chemistry, end-chain reactive chemistry, branched-chain reactive chemistry and the like, as well as any bioconjugation or covalent chemistry that would be ordinarily understood by a skilled artisan. Depending upon the desired substrate, the anchor can merely be a substrate that directly reacts with the surface-mutated site and need not have additional “guide” components for guiding site-specific surface interactions (e.g. upon a zwitterionic surface).

A combination of electrostatic ligands for stabilizing gRNA/dsDNA/ssDNA/oligonucleotides may also be used with covalently bonded or PNA-bound conjugations to modified surface-exposed residues on a ribonucleoprotein or protein. This affords the advantage of spatially controlling ligand (or cell-penetrating-peptide, stealth motif, stabilizing motif, and the like) assembly, and allowing for multifunctional patterning of ligands, endosomolytic motifs, subcellular trafficking motifs, and the like upon a minimally-modified recombinant protein (modRNP, modProtein, and or modLigands).

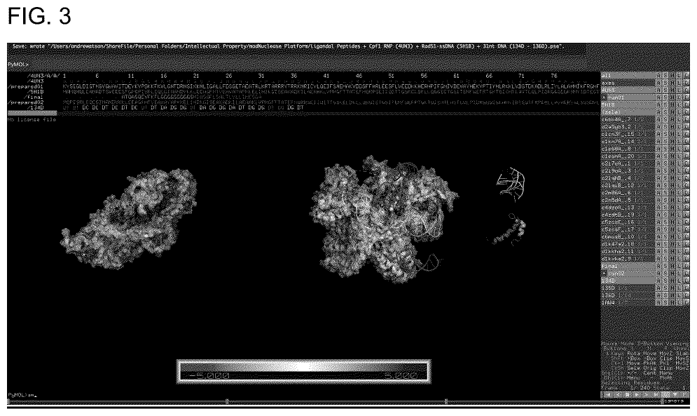

FIG. 3 depicts a three-dimensional model a Rad51-DNA complex (left), Cpf1 RNP (middle), 31nt DNA (top right), and an exemplary anchor-linker-ligand (bottom right). Concomitant to this approach, Rad51 (left; or Rad54), or a similar DNA-binding protein, may be used to bind an ssDNA or dsDNA to a recombinantly modified nuclease. The ssDNA or dsDNA may also be directly 3′ or 5′ covalently linked, or alternatively a PNA/RNA/DNA/LNA/MNA may be covalently linked to the desired protein surface, with or without Rad51/Rad54. Multiple conjugation sites for annealing or directly covalently tethering a nucleic acid species' 3′ or 5′ end may be used to create a homogenously negative charged corona around the protein or RNP surface, and to drive higher rates of homology-directed repair than single copies. An embodiment of the disclosure does not necessarily need to have multiple copies of ssDNA/dsDNA/tetrisDNA, so much as one or more copies that either directly neutralize a cationic domain of the electrostatic protein/RNP surface upon conjugation, or electrostatically neutralize a cationic domain and/or facilitate assembly via electrostatic and/or lipid and/or click chemistry and/or bioconjugation chemistry and/or other self-assembling nanoparticle/layer-by-layer techniques. Modified Rad51/Rad54 in this example may have anionic charge sites and non-critical residues modified to neutral or maintained as anionic patches. In this example, DNA-binding sequences upon a modRNP or modProtein, and sequences necessary for activity with subsequent DNA repair machinery, are not altered in the mutagenesis process unless the process is catalytically enhancing for HDR and DNA repair.

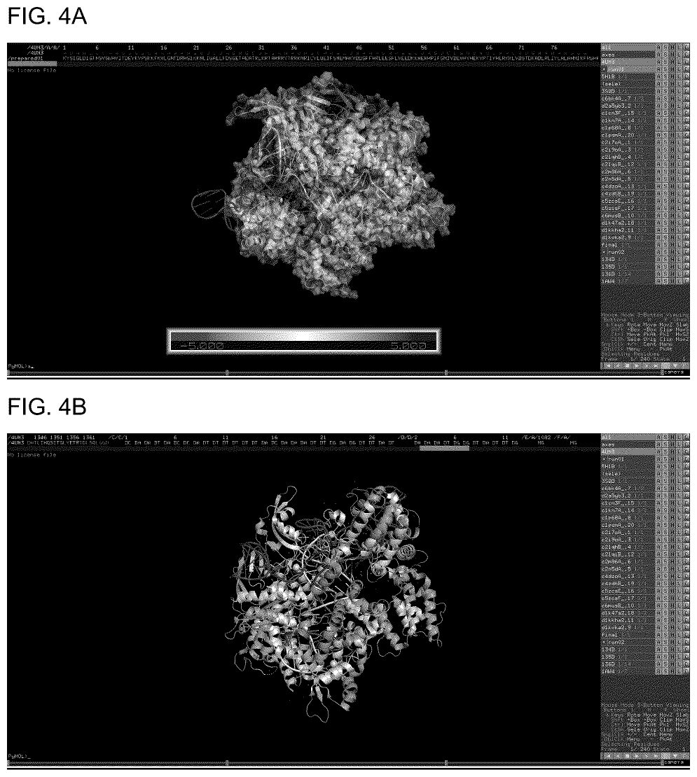

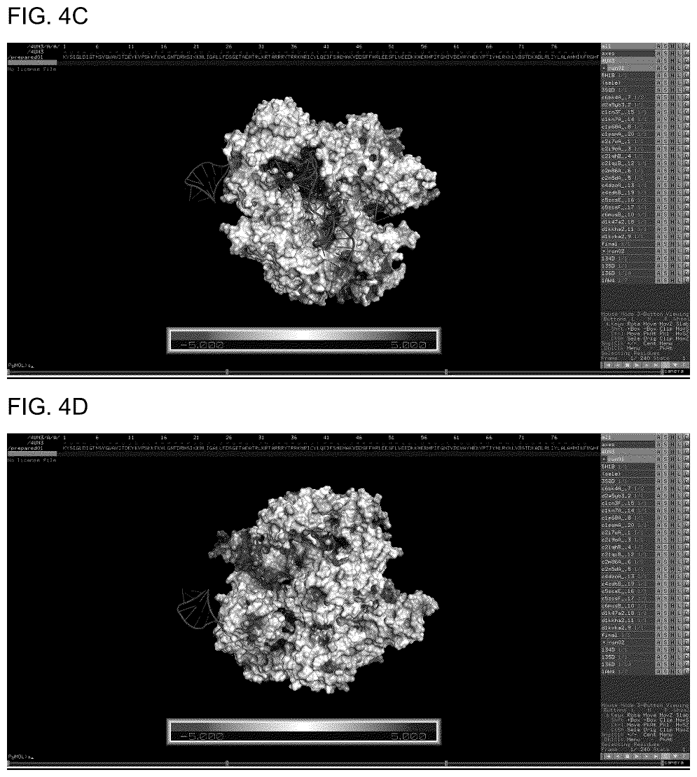

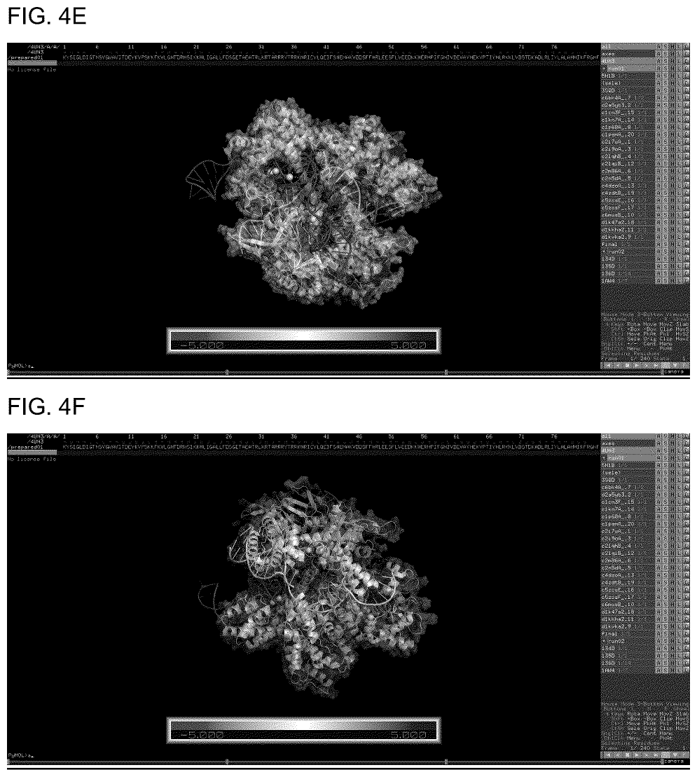

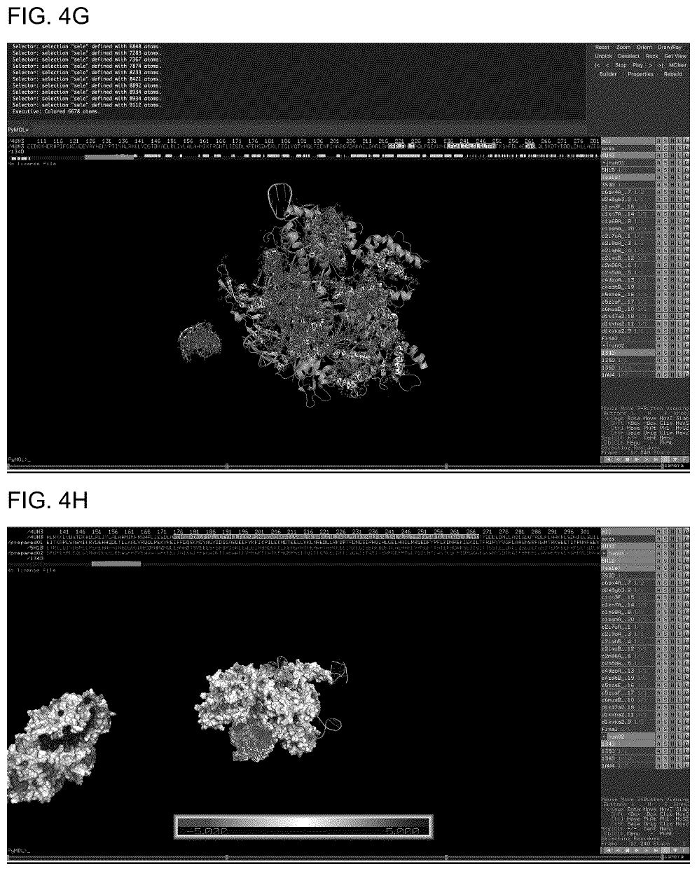

Also modeled in the present example is Cpf1 selective mutagenesis, whereby non-surface-exposed and/or catalytically-important inwardly-facing residues are maintained in their wildtype state. FIGS. 4A-4H. Exposed residues and non-critical residues for activity are highlighted in green. Overlay with Poisson-Boltzmann plots allows for determination of key surface mutagenesis sites, including ranges of amino acids (FIG. 4H, P175-D268) that represent charged non-activity-critical patches. These patches may be cationic, neutral or anionic prior to selective mutagenesis, so long as the resulting coupling via, for example, covalent modification, bioconjugation, electrostatic interactions, annealing-based coating, or tethering is facilitated by a polymer chain (including nucleic acids and nucleic acid-like molecules) being coupled with the mutated amino acid(s). Multivalent presentation of ssDNA/dsDNA may also enhance genomic integration fidelity, while stabilizing the protein surface anionically, directly, or indirectly as discussed herein.

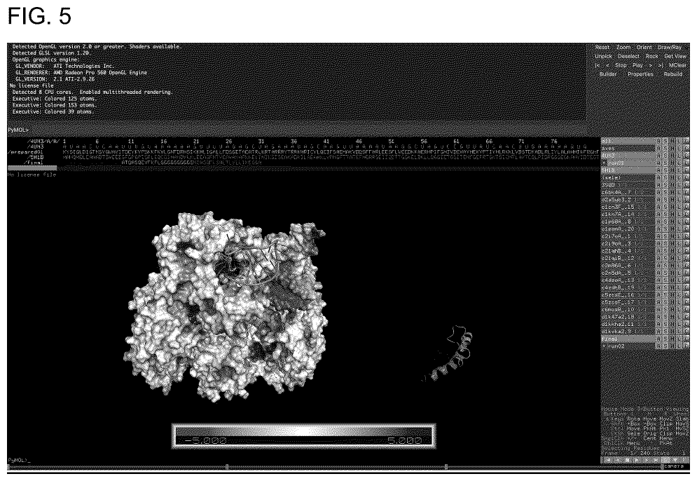

Additional modeling in FIG. 5 depicts Cpf1-gRNA complex (left) including an anchor-linker-ligand (cationic H2AX-derived [119-143] anchor in blue), linker (green), cleavable domain (orange), and ligand (pink) (right). In this model, the cationic portion of anchor-linker-FKFL-ligand may fold out and preferentially bind to any anionic residues on the zwitterionic protein surface (or other substrate), and excessive self-folding can be minimized through manipulation of linker lengths and rigidity as modeled elsewhere. These embodiments include variants where the protein or coating or underlying layer is a non-zwitterionic substrates or part of any layer-by-layer assemblies as would be reasonably understood by one ordinarily skilled in the art). The anchor's length can be modulated (extended or reduced) to match charged pockets (including through multiple anchors being utilized in various molar ratios), as well as pockets with various degrees of hydrophobicity/hydrophilicity with corresponding anchor phases. Additionally, lipid bilayer embodiments through polymer-lipid and peptide-lipid fusions may be utilized to form bilayers, as with ceramide-6-phosphate being carbodimidine-linked to an amide of a peptide or two or more peptides, whereby self-assembly behaviors of the electrostatic “core-facing” phase of the bilayer may be dictated through what motifs and charges are present on the internal surface of the bilayer, and the cell-targeting and cellular-affinity-generating behaviors of the “outwardly-facing” phase of the bilayer may be dictated through modulation of ligands and/or cell-penetrating peptides and/or charge on the particle surface (see, e.g., Yamano 2016). Modeled in FIGS. 6A-B is Rad51 bound to an ssDNA molecule (left) and Cpf1 with Poisson-Boltzmann electrostatic surface potential plots (overlay), in complex with sgRNA (right). See https://apbs-pdb2pqr.readthedocs.io/en/latest/releases/201601-APBS-1.4.2.1.html (regarding Poisson Boltzmann).

### Example 2—Nanoparticle Size Analysis (Modification/Buffers/Charge Homogenization)

Previous studies demonstrated that Cas9 RNPs by themselves are ˜100 nm, and even after a variety of buffer optimization conditions, remain in an aggregated state despite individual RNPs being ˜12 nm in diameter. See U.S. Provisional Appl. No. 62/842,400.

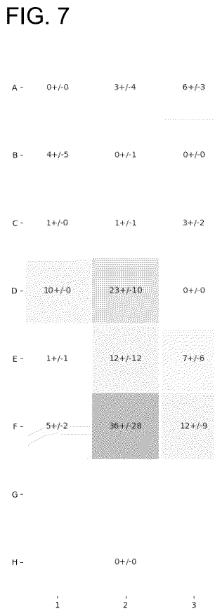

FIG. 7 depicts various buffers and pH conditions that may be utilized for achieving efficient electrostatic nanoparticle condensation (left), and associated intensity profiles of Cas9 RNPs in the 1-20 nm range (right) prior to nanoparticle formation. Table 1 (below) includes the respective buffer (A-H) and pH conditions (1-3) used for each cell in FIG. 7. Prior to optimization of Cas9 “core RNP” sizes, Cas9 aggregates are formed in the ˜70-100 nm range. Optimization of buffer conditions yields acceptable RNP sizes for subsequent nanoparticle formation, despite being substantially larger than individual Cas9 RNPs (˜12 nm hydrodynamic diameter in non-aggregated state). pH 6.5 1×PBS and 25 mM pH 6.5 HEPES yielded optimal Cas9 RNP sizes for subsequent layering of RNPs.

TABLE 1

| | | 1 | 2 | 3 |
|---|---|---|---|---|
| 100 mM Bis Tris | A | pH 5.5 | pH 6.5 | pH 7.5 |
| 10 mM Tris | B | pH 7.0 | pH 8.0 | pH 9.0 |
| 100 mM Tris | C | pH 7.0 | pH 8.0 | pH 9.0 |
| 25 mM HEPES | D | pH 5.5 | pH 6.5 | pH 7.5 |
| 100 mM Ammonium Tartate | E | pH 5.5 | pH 6.5 | pH 7.5 |
| 1x Phosphate-Buffered Saline (PBS) | F | pH 5.5 | pH 6.5 | PH 7.5 |
| 100 mM Sodium Citrate | G | pH 5.5 | pH 6.5 | pH 7.5 |
| 100 mM Sodium Acetate | H | pH 5.0 | pH 6.0 | pH 7.0 |

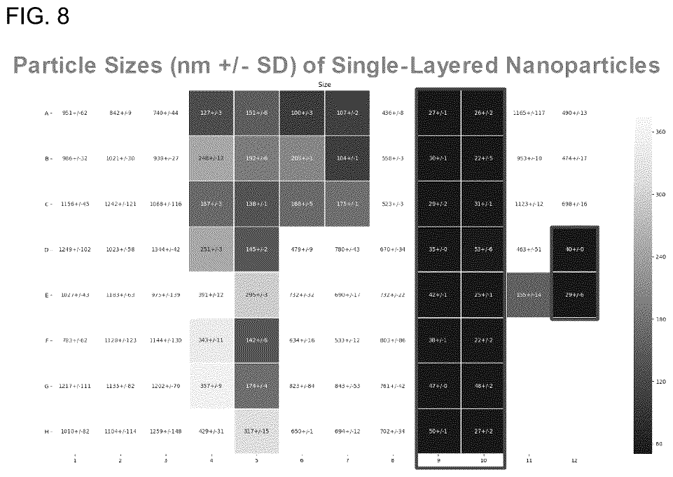

FIG. 8 depicts particle sizes of CRISPR RNP following a number of electrostatic self-assembling nanoparticle formulations. Shown here are particle sizes of each respective single-layered nanoparticle formulation as shown in U.S. Provisional Appl. No. 62/842,400, demonstrating that PLR10 and similarly-sized cationic polymers can be used to neutralize anionic charge pockets on a zwitterionic protein surface to allow for optimized layer-by-layer nanoparticle formation. In this example, PLK10-PEG22 and PLR10 particles with variable endosomal escape peptide/functional domain peptide (EE) concentrations are shown to condense NLS-Cas9-NLS, but not NLS-Cas9-EGFP, into sub-50-nm particles at 3 orders of addition of EE vs. cationic polypeptide groups (wells A9-H10 and D12-E12). These particle sizes are demonstrably smaller than RNP-only sizes and suggest the role of short (<20 AA) cationic polypeptides in being able to uniquely dissociate RNP aggregates prior to subsequent multilayering or inclusion with a variety of nanoparticle formulations or alternative delivery systems (e.g., covalently modified RNPs, liposomes, and the like). We have previously demonstrated nanoparticles condensed in this way to be multilayered with either another nucleotide and PLE/PDE, or a nucleotide on its own, prior to a final layer of cationic anchor-ligand. We have also demonstrated anionic anchor-ligand groups to be able to condense around cationic layers. This screening study demonstrates iterative cell-specific ligand design whereby individual ligands are interrogated and optimized at various densities and with various core templates. Additionally, this allows for ligands to be modularly studied upon a variety of core chemistries and polymer/polypeptide compositions, as well as various payloads. Compared to the heteromultivalent studies (where a global optimal was found for a static set of targeting ligand densities, e.g., anchor cationic interactions with anionic payload or vice versa), these results demonstrate that further core optimization may also achieve optimization of cellular uptake and affinity of single ligands for various cell subpopulations. Ligand-coated complexes outperform cell-penetrating peptide coated complexes.

Studies with these approaches have been used for mRNA co-delivery in previous experiments (e.g., CynoBM.002 in PCT/US17/66545) whereby >97% cellular colocalization of Cas9-EGFP RNPs and Cy5 mRNA was observed, and whereby these smaller nanoparticles consisting of dissociated Cas9 RNP aggregates coated in short cationic polymers are successfully coated in mRNA and/or anionic polymers prior to functionalization in a cationic anchor-linker-ligand as part of an electrostatic codelivery strategy. In certain embodiments, smaller nanoparticles include nanoparticles that are <100 nm in size, <70 nm in size, or less than the size of native Cas9 RNP aggregates. For example, nanoparticles <70 nm in size are required to engage caveolae- or clathrin-mediated and/or receptor-mediated endocytic pathways which preferentially sort away from lysosomes. These embodiments are non-limiting, as an anionic-linker-ligand may be used in place of an anionic “layer,” and “layer-by-layer” assembly is not necessary when a protein is “charge homogenized” by the methods and techniques disclosed herein. Furthermore, use of an anionic polynucleotide as a 3′ or 5′ conjugated or linked compound to a protein substrate's otherwise cationic charge patches may be an effective method for charge neutralization that requires study of structure-function of the target protein, its surface potential, and the desired genome engineering/bioengineering feat to be performed.

In certain embodiments, free RNP may serve as seed substrates for subsequent nanoparticle formation, in contrast to RNA/DNA-cationic peptide interactions where there is no “seed substrate.” In certain preferred embodiments, presenting an as-small-as-possible RNP size at the time of nanoparticle formation may yield optimal nanoparticle properties (including <70 nm variants) that may be particularly well suited for caveolae-mediated and clathrin-mediated receptor-specific endocytic pathways due to endosomal vesicle sizes >70 nm preferentially accumulating in lysosomal and phagocytic pathways. Engagement of “long endosomal recycling pathways” and “short endosomal recycling pathways” may be utilized to optimize nanoparticle uptake into endosomal vesicles that may possess enhanced subcellular trafficking pathways for cytosolic and nuclear delivery of a variety of payloads, and these specific endosomal pathways are not present when nanoparticle sizes are sufficiently large. Optimization of seed substrate size is a key component of finding optimal nanoparticle formulations for cell-specific cellular transfection.

The methods and techniques provided herein for modifying zwitterionic pockets of Cas9 can be utilized to create non-aggregated Cas9 for subsequent layer-by-layer assembly (or single-step chemical conjugation and/or electrostatic polymer and/or electrostatic polymer-PEG and/or electrostatic polymer-linker and/or any ligand-bearing variants thereof.

Previous studies demonstrate the “charge homogenization” hypothesis with PLR10 and PLK10-PEG22, which are uniquely able to dissociate aggregates of Cas9 in previous experiments. See PCT/US17/66541, PCT/US17/66545, U.S. Provisional Appl. No. 62/842,400. Larger molecular weight polymers such as PLR50, and even 9R-GGGGSGGGGS-ligand variants, were not able to mediate the same spontaneous dissociation of Cas9 RNP aggregates.

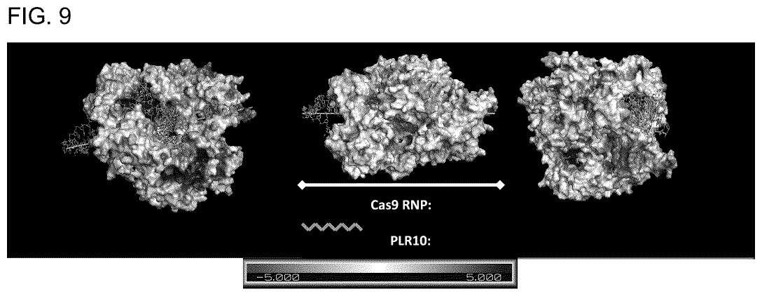

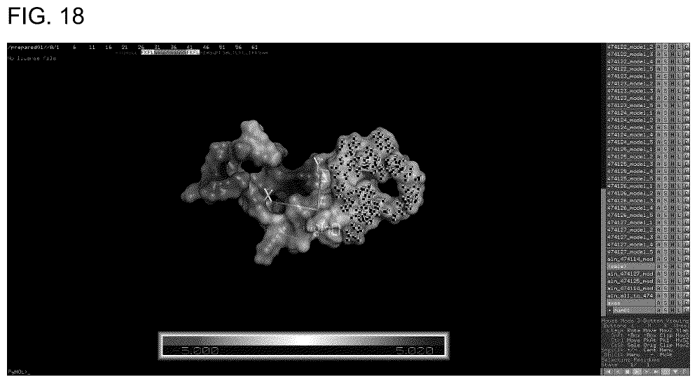

FIG. 9 depicts size considerations examining why poly(L-arginine) (n=10) and PLK10-PEG22 consistently formed CRISPR RNP nanoparticles in the 20-59 nm range. It is hypothesized that PLR10 and PLK10-PEG22-which have polymer chain lengths less than the hydrodynamic diameter of Cas9 RNP-will preferentially “charge switch” the anionic components of the highly zwitterionic Cas9 RNP. Methods of using “charge switching” techniques for achieving affinity of peptide sequences to zwitterionic surfaces are also detailed in FIGS. 18T and 18U. If PLR10 or similarly sized cationic polypeptides can intercalate into the anionic pockets of the zwitterionic protein, the otherwise aggregative properties of Cas9 (likely due to opposite charges interacting and forming electrostatic aggregates) may be reversed. These small, homogenously-charged cationic RNP-PLR10 complexes may be subsequently decorated in a variety of surface coatings, including anionic interlayers (e.g., PLE/PDE) with or without subsequent cationic anchor-linker-ligand or anchor-peptide sequences, as well as anionic anchor-linker-ligand or anchor-peptide sequences. Additionally, PLR10 serves to efficiently condense exposed sgRNA residues of the Cas9 RNP, which are anionic in nature.

In sum, modification of the Cas9 or other appropriate seed substrate's (e.g., nuclease, protein) key anionic/cationic patches may be performed for creating homogenous self-assembly of oppositely-charged polypeptides. It is non-obvious to modify a zwitterionic protein in this way to aid self-assembly. It is also non-obvious to perform one or more ssDNA/dsDNA tetherings to such a modified protein to provide the anionic potential necessary for optimal layering, and such layer-by-layer assembly is not enhanced merely by mixing larger ssDNA/dsDNA cassettes with Cas9 RNP prior to introduction of cationic polymers/polypeptides, in contrast to the increases in efficiency seen when sgRNA:Cas9/Cpf1 ratios are >1:1 whereby the sgRNA electrostatically neutralizes some of the cationic surface potentials on Cas9.

Additionally, according to embodiments according to the present disclosure, a cationic site need not be modified into an anionic site, and an anionic site need not be modified into a cationic site. So long as spacing of where the respective charged (or alternative anchor) site presents adequate sites for conjugation to the subsequent moieties for nanoparticle formation (e.g., molecule, motifs, nanoparticle layers, PNA, DNA, RNA, peptide, polymer etc.).

This novel approach allows for the donor DNA and/or polymers, peptides, etc. to serve as “charge homogenizing” and/or aggregation-limiting substrates for subsequent electrostatic, covalent, or other affinity-guided assembly for self-assemblable nanoparticle design and formation.

### Example 3—Predictive Modeling of E-Selectin Ligand and Novel “Perfect Insertion” DNA-Rad51-Fusion-Cas9-RNP Simulation

The present example provides an illustration of predictive modeling for use in “perfect insertion” of an e-selectin ligand using a DNA-Rad51-fusion-Cas9-RNP modeled according to embodiments of the present disclosure. Previous studies demonstrate that histone H2AX enhances DNA repair through phosphorylation of AA140 (See Sofueva 2010; Yan 2011). The crystal structure of human PTIP BRCT5/6-gamma H2AX complex has been described previously (See PDB ID: 3SQD, DOI 10.2210/pdb3SQD/pdb (https://www.rcsb.org/structure/3sqd)). Sequences used in the present example are described in Table 2, below. Additional histone modifications not presented in Table 2, such as those described previously are also contemplated (See, e.g., Clouaire 2018).

TABLE 2

| Sequence(s) | Description |
|---|---|
| KKRTSATVGPKAPSGGKKATQASQEY | Histone H2AX [119-143] (“anchor domain”) |
| (SEQ ID NO: 13) | |
| [GGGGS]n | Representative “stiff” linker domain that |
| | can be substituted for any helical-forming |
| | domain or other rigid domain (optional |
| | “linker domains” 2) |
| [GAP]m | Representative “stiff” linker domain that |
| | can be substituted for any helical-forming |
| | domain or other rigid domain (optional |
| | “linker domains” 2) |
| GAPGPSGARGERGFPGERGVQG | Non-GAP and non-GGGGS sequences |
| (SEQ ID NO: 14) | GAPGPSGARGERGFPGERGVQG are |
| | derived from ColA1 and are a |
| | representative Gly-X-Y domain (optional |
| | “linker domains” 3) |
| FKFL (SEQ ID NO: 3) | Cathepsin B sensitive proteolytic cleavage |
| | domain, necessary for exposing |
| | phosphorylated AA140 of H2AX to DNA |
| | repair machinery (optional “cleavable |
| | domain”; may also be an S—S or Se—Se |
| | bond and the like) |
| MIASQFLSALTLVLLIKESGA | An E-selectin ligand [1-21] (optional “ligand |
| (SEQ ID NO: 5) | domain”) |

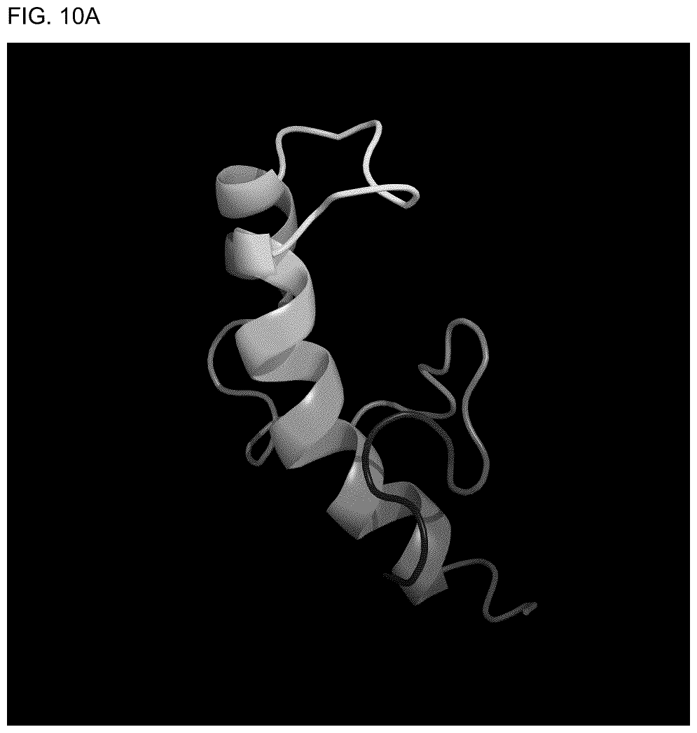

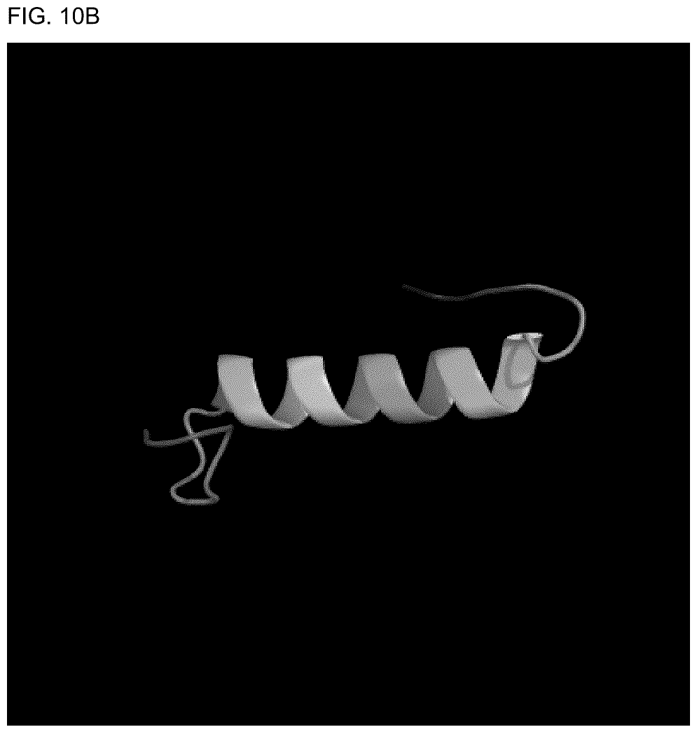

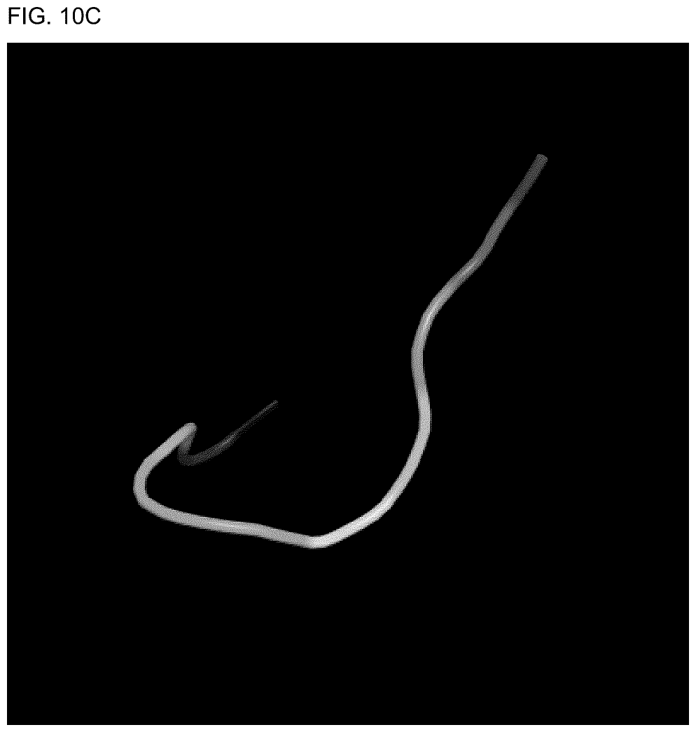

In this example, FIGS. 10A-10C depict three sequence-alignment based simulations of the sequence KKRTSATVGPKAPSGGKKATQASQEYFKFLGGGGSGGGGSFKFLMIASQFLSALTLVLLI KESGA (representative anchor-linker-ligand; SEQ ID NO:1), whereby Phyre2 was used to model the sequence. The N-terminus (red) is anticipated to have 6 cationic residues that can intercalate with anionic components of a preceding layer or associated component. The N-terminus may also be modified with a PNA, RNA, DNA, or the like in order to serve as a complementary affinity bind for a previous layer's PNA/RNA/DNA/etc. complementary sequence. The C-terminus (blue) represents an E-selectin ligand, and as with any ligand <20AA in length the structure is expected to be a random coil with an “induced fit” upon binding to the appropriate receptor or protein/other target. An antibody, ScFv, aptamer and the like may also be presented following the linker domain or directly extended off the anchor domain, which need not be an electrostatic anchor domain. The corresponding pdb files for these two images are “final.pdb,” “c6bk4A 0.7.pdb,” and “d1kvka2.9.pdb,” respectively. A random coil such as with d1 kvka2.9 may be utilized to approximate the anchor, linker and/or ligand length that would be most appropriate for forming a strong binding surface to the underlying protein substrate (covalent or non-covalent), in addition to the appropriate length of an optional spacer (and its associated secondary structure esp. with >20AA), as well appropriate length of an optional ligand (and/or selective mutagenesis) for maintaining optimal ligand conformation upon receptor binding in the event that an “induced fit” hypothesis is not sufficient.

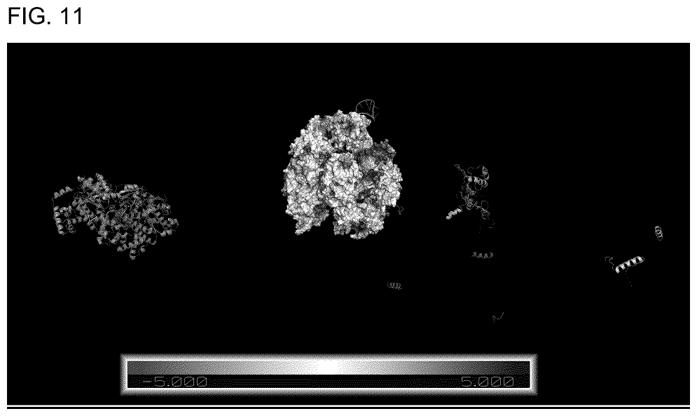

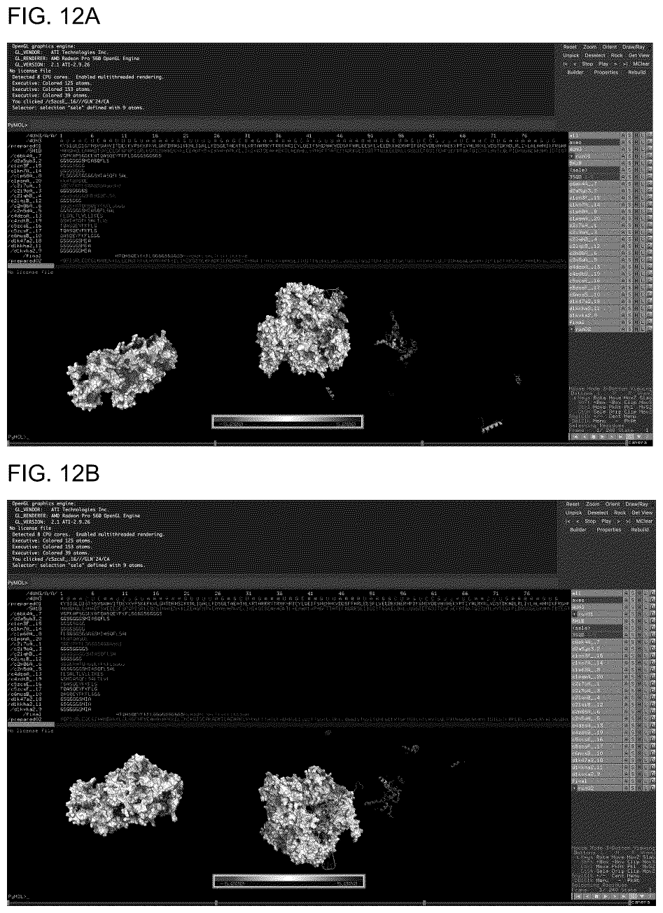

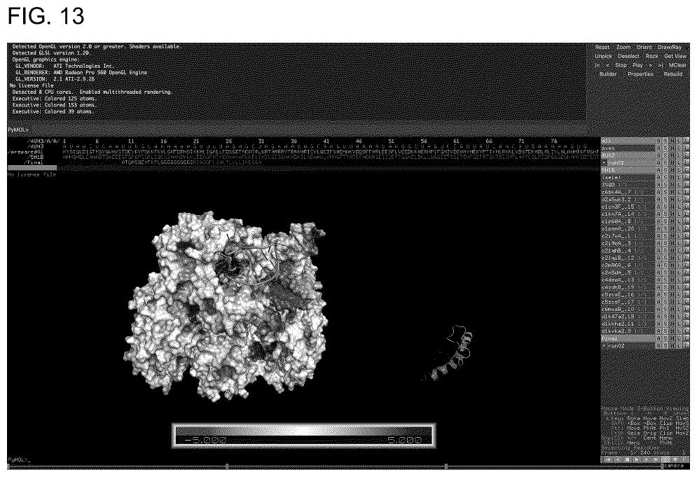

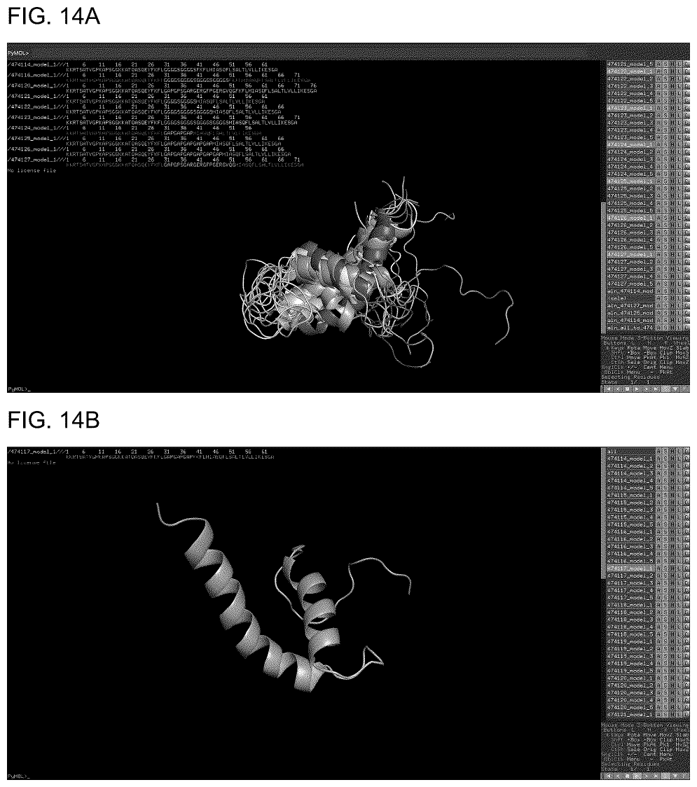

In further modeling in this example was performed to examine a comparison of overall predicted anchor-linker-ligand structures for KKRTSATVGPKAPSGGKKATQASQEYFKFLGGGGSGGGGSMIASQFLSALTLVLLIKESG A (SEQ ID NO:2), to scale, with Cpf1 RNP (middle) and Rad51-ssDNA complex (left) shown. See FIG. 11. Anchors may be tailored in length to match electrostatic pockets, may have multiple anchor lengths, and may also be tethered covalently or through alternative techniques as described herein. Additionally, lipid-based embodiments may be used to facilitate bilayer formation between an electrostatic or otherwise tethered layer with affinity for the lipid (including anionic lipids, cationic lipids, PNA-lipids, and the like). FIGS. 12A and 12B depict Rad51 (left), Cpf1 RNP (middle), and a variety of relevant anchor, linker and/or ligand sequences as modeled by Phyre2. The “final” ligand simulation in this example is presented in FIG. 13, which depicts a multi-domain peptide sequence whereby the blue electrostatic domain (which may have various extensions of histidines, arginines and/or lysines, NLSs, and the like on its N-terminus or throughout the H2AX-derived sequence), cleavable domain (FKFL; SEQ ID NO:3), linker domain (GGGGSGGGGS; SEQ ID NO:4), and E-selectin ligand domain (MIASQFLSALTLVLLIKESGA; SEQ ID NO:5). Linkers may also be bioconjugatable to various ScFvs, biologics, aptamers, and the like in place of a peptide, glycopeptide or lipopeptide ligands. Prediction of charged patches as well as associated anchor lengths and phases allows for forming spatially predictive nanoparticle assemblies.

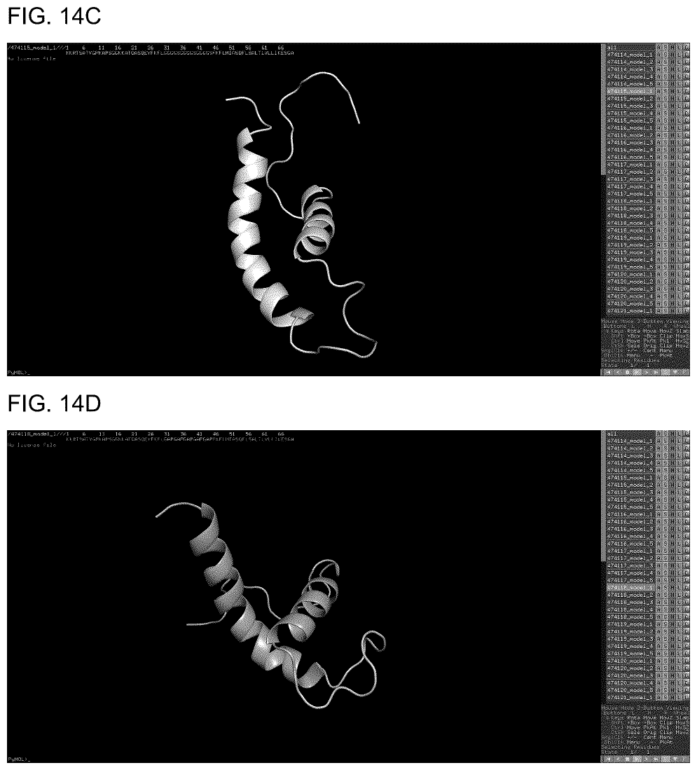

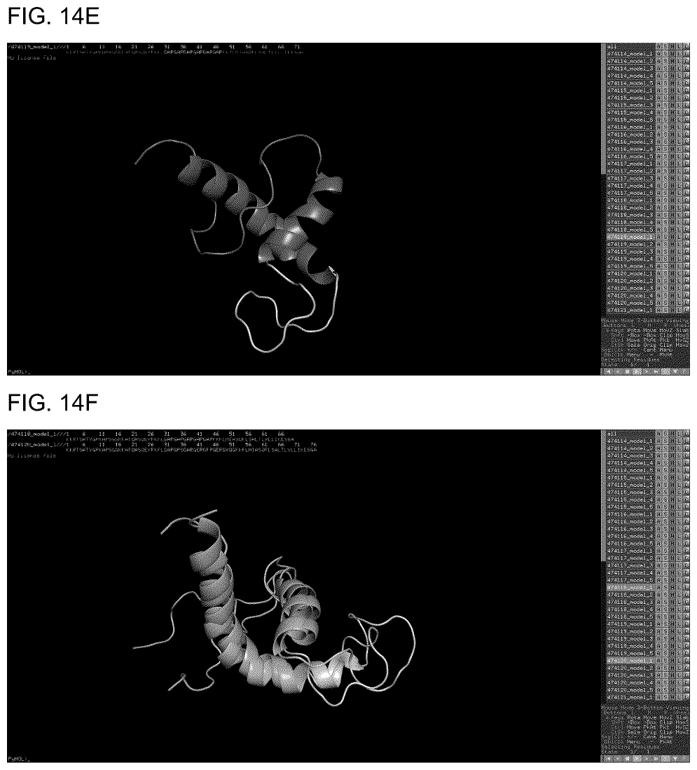

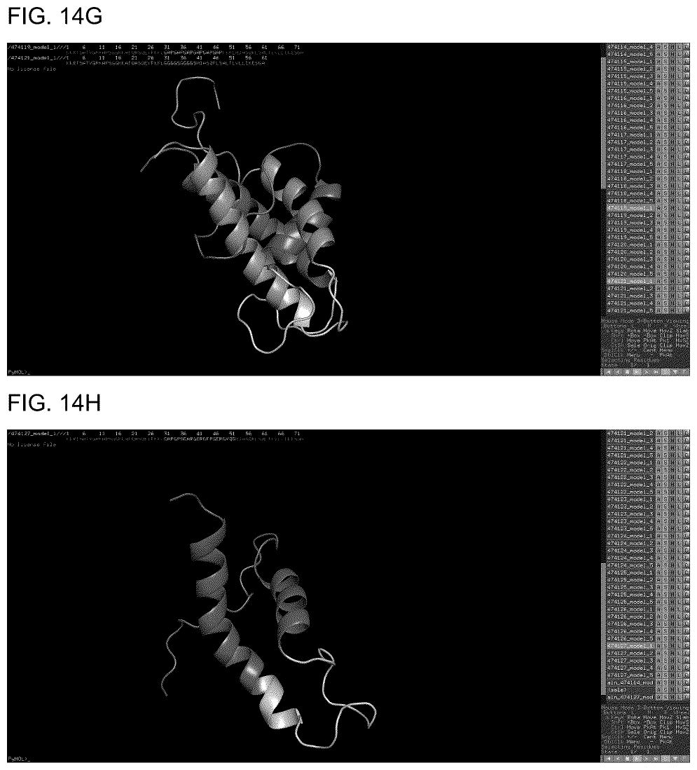

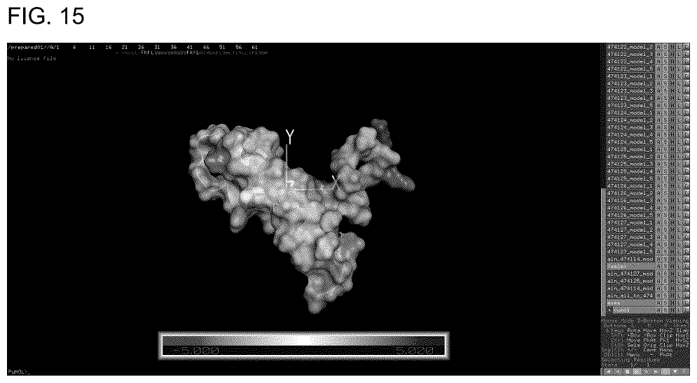

Modeling was additionally used to predict the alignment of various self-assemblable nanoparticle components including anchors, linkers, and ligands. FIGS. 14A-14H depicts ligand domain (E-selectin ligand, pink), cleavable domains (FKFL, yellow), linker domain (GGGGSGGGGS (SEQ ID NO:4), GAPGAPGAP (SEQ ID NO:6), and the like, orange), and anchor domain (histone H2AX-derived domain with optional S139 phosphorylation, blue) with partially transparent Poisson-Boltzmann plot overlaid. Cleavable domain facilitates exposure of phosphorylated S139 to DNA repair machinery, while the H2AX domain preferentially remains electrostatically and/or covalently and/or otherwise bound/linked to the protein surface and promotes nuclear targeting and/or neutralization of charge potential between one or more histone domains and any electrostatic sequences (e.g. nucleic acids) within the carrier. Modeling in this example was additionally used to visualize the ligand domain (E-selectin ligand, pink), cleavable domains (FKFL (SEQ ID NO:3), yellow), linker domain (GGGGSGGGGS (SEQ ID NO:4), GAPGAPGAP (SEQ ID NO:6), and the like, orange), and anchor domain (histone H2AX-derived domain with optional S139 phosphorylation, blue) with partially transparent Poisson-Boltzmann plot overlaid. See FIG. 15. Cleavable domain facilitates exposure of phosphorylated S139 to DNA repair machinery, while the H2AX domain preferentially remains electrostatically and/or covalently and/or otherwise bound/linked to the protein surface and promotes nuclear targeting and/or neutralization of charge potential between one or more histone domains and any electrostatic sequences (e.g., nucleic acids) within the carrier.

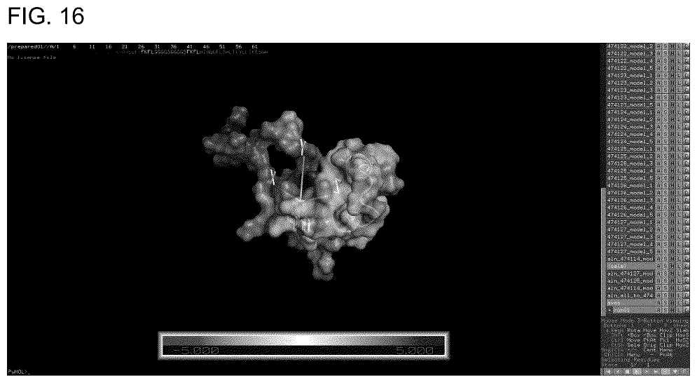

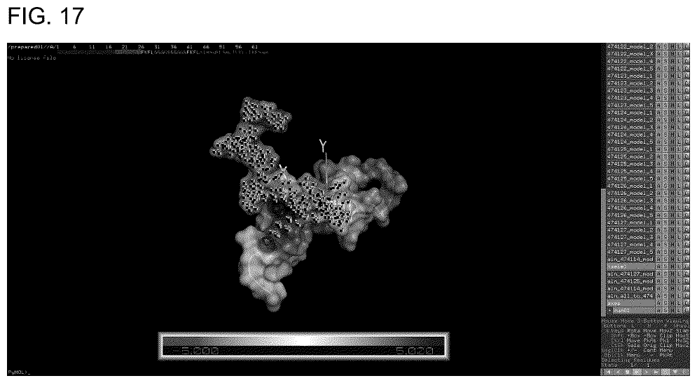

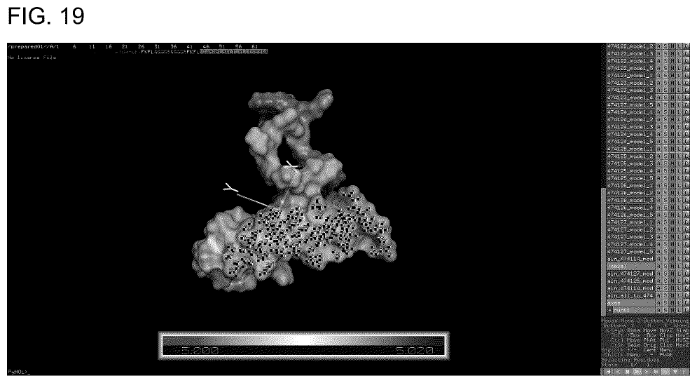

Modeling in this example also utilizes Poisson-Boltzmann plot overlays as described herein. For example, FIG. 16 depicts further modeling of an E-selectin ligand, cleavable linker, and anchor with partially transparent Poisson-Boltzmann plot overlaid. Large cationic surface potential areas (blue) will preferentially dissociate from their self-folding behavior following interaction with an anionic surface potential, nucleic acid, amino acid, and the like. An anionic anchor, PNA/DNA/RNA-affinity anchor, and/or other “clamping” mechanism (either through reducibly cleavable/covalent chemistry, or otherwise, but also including electrostatic/alternative-phase-based-mimicry self-assembly) may be used. In FIG. 17, depicts H2AX-derived (119-143) anchor domain with optional extensible electrostatic domain (1-18) that may be modified with additional histone domain fragments, KKR motifs, NLS, cationic charges, anionic charges, hydrophobic AA, hydrophilic AA, hydrogen bonding AA, and the like; including Phosphorylated and otherwise post-translationally modified (see, e.g., U.S. patent application Ser. Nos. 15/842,820, 15/842,829, 16/387,507, 16/393,886, and 62/842,400) variants thereof and H4-derived histone variants thereof (highlighted), as well as any other factors or fragments thereof that may promote DNA repair or cell-cycle dependent processes. A partially transparent Poisson-Boltzmann plot is overlaid. FIG. 18 depicts cleavable linker domain (highlighted) with partially transparent Poisson-Boltzmann plot overlaid and FIG. 19 depicts E-selectin ligand domain (highlighted) with partially transparent Poisson-Boltzmann plot overlaid.

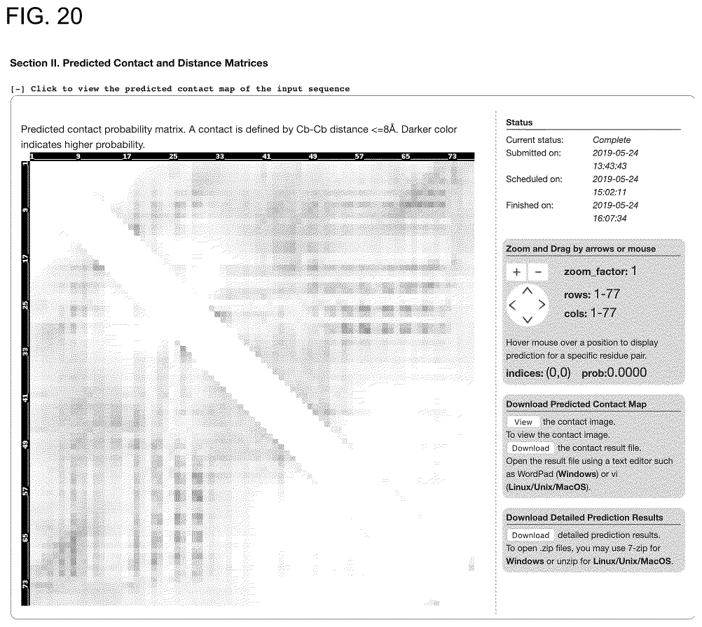

FIG. 20 depicts predicted contact and distance matrices, where darker boxes indicate more interaction. The anionic or cationic electrostatic or alternative protein-affinity-phase-mimetic domain (in this embodiment, H2AX fragment, but also possibly hydrophobic, hydrophilic, hydrogen bonded and the like) will “unfold” if it shares an interactive set of motifs following binding to a protein or other (e.g., nucleic acid, charged) substrate which carries higher affinity for the anchor than the anchor does to the linker and/or ligand (“self-binding” and “unfolding domain”).

### Example 4—Predictive Modeling of CD34 Targeting

Predicative modeling techniques according to the present disclosure may be used to visualize self-assemblable nanoparticle components. In this Example, these predictive modeling techniques have been used to visualize and predict binding to CD34 domains to target CD34.

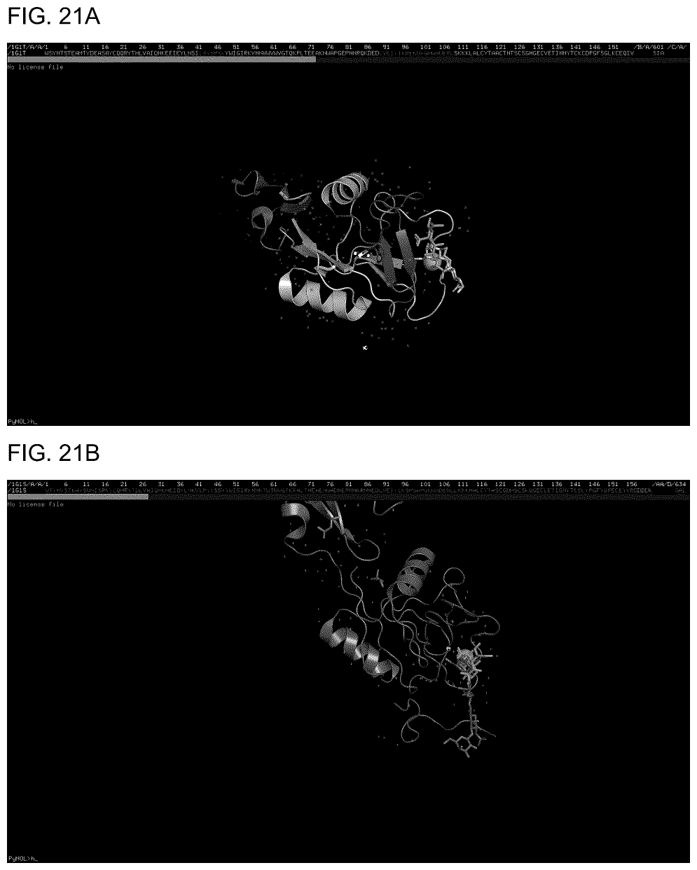

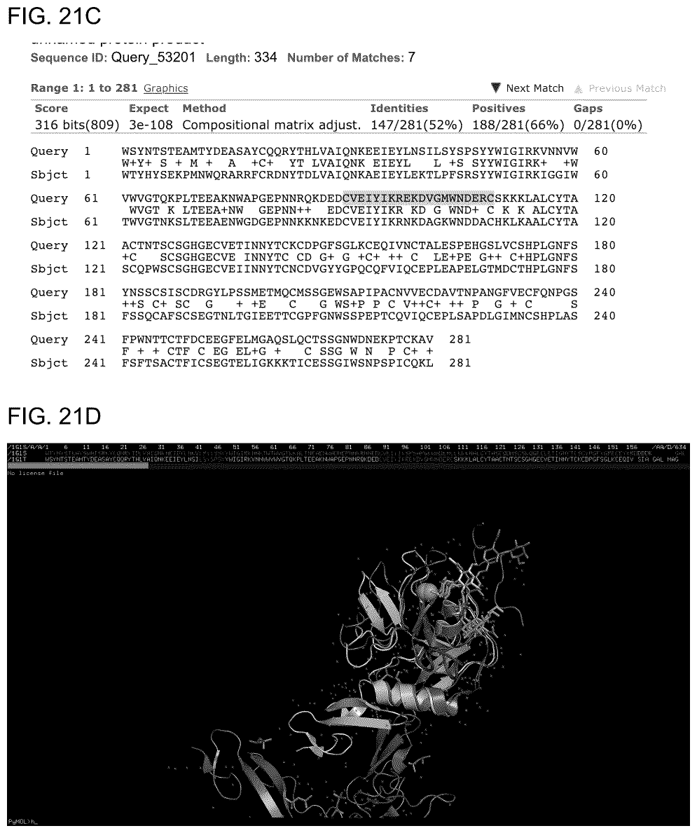

In one aspect, the conserved L-selectin and E-selectin binding domains binding to sialyl Lewis X were modeled. FIGS. 21A and 21B are three dimensional models depicting PDB ID: 1G1T (E-selectin) bound to sialyl Lewis X (SIA) (FIG. 21A) and PDB ID: 1G1S (L-selectin) bound to SIA (FIG. 21B).

Applicant used sequence alignment to align portions of E-selectin and L-selectin included in Table 3 below. Sequence alignment of E-selectin (22-610) and L-selectin (39-332) is depicted in FIG. 21C.

TABLE 3

| Gene | Sequence |
|---|---|
| E-selectin | >sp\|P16581\|22-610 |
| (22-610) | WSYNTSTEAMTYDEASAYCQQRYTHLVAIQNKEEIEYLN |
| | SILSYSPSYYWIGIRKVNNVWVWVGTQKPLTEEAKNWAP |
| | GEPNNRQKDEDCVEIYIKREKDVGMWNDERCSKKKLALC |
| | YTAACTNTSCSGHGECVETINNYTCKCDPGFSGLKCEQI |
| | VNCTALESPEHGSLVCSHPLGNFSYNSSCSISCDRGYLP |
| | SSMETMQCMSSGEWSAPIPACNWVECDAVTNPANGFVEC |
| | FQNPGSFPWNTTCTFDCEEGFELMGAQSLQCTSSGNWDN |
| | EKPTCKAVTCRAVRQPQNGSVRCSHSPAGEFTFKSSCNF |
| | TCEEGFMLQGPAQVECTTQGQWTQQIPVCEAFQCTALSN |
| | PERGYMNCLPSASGSFRYGSSCEFSCEQGFVLKGSKRLQ |
| | CGPTGEWDNEKPTCEAVRCDAVHQPPKGLVRCAHSPIGE |
| | FTYKSSCAFSCEEGFELHGSTQLECTSQGQWTEEVPSCQ |
| | VVKCSSLAVPGKINMSCSGEPVFGTVCKFACPEGWTLNG |
| | SAARTCGATGHWSGLLPTCEAPTESNIPLVAGLSAAGLS |
| | LLTLAPFLLWLRKCLRKAKKFVPASSCQSLESDGSYQKP |
| | SYIL |
| | (SEQ ID NO: 15) |
| L-selectin | >sp\|P14151\|39-332 |
| (39-332) | WTYHYSEKPMNWQRARRFCRDNYTDLVAIQNKAEIEYLE |
| | KTLPFSRSYYWIGIRKIGGIWTWVGTNKSLTEEAENWGD |
| | GEPNNKKNKEDCVEIYIKRNKDAGKWNDDACHKLKAALC |
| | YTASCQPWSCSGHGECVEIINNYTCNCDVGYYGPQCQFV |
| | IQCEPLEAPELGTMDCTHPLGNFSFSSQCAFSCSEGTNL |
| | TGIEETTCGPFGNWSSPEPTCQVIQCEPLSAPDLGIMNC |
| | SHPLASFSFTSACTFICSEGTELIGKKKTICESSGIWSN |
| | PSPICQKLDKSFSMIKEGDYN |
| | (SEQ ID NO: 16) |

FIG. 21D depicts a three-dimensional model of the alignment of 1G1T (E-selectin) and 1G1S (L-selectin). Based on this model, Applicant has identified key domains and binding sites to sialyl Lewis X that remain identical. These sequences are LPYYSSY (SEQ ID NO:17), LSYSPSY (SEQ ID NO:18), CVEIYIKSPSAPGKWNDEHC (SEQ ID NO:19), and CVEIYIKREKDVGMWDERC (SEQ ID NO:20) (cyclical peptides) and are predicted to be ideal for binding of E- and L-selectin to sialyl Lewis X and CD34. See FIG. 21D.

Sialyl Lewis X and other complex carbohydrate structures may be modified and coupled to a peptide or polymer sequence using peptide synthesis chemistry and N-to-C coupling techniques involving typical protection and deprotection chemistry (e.g., fluorenylmethyloxycarbonyl (Fmoc), Benzyl-ester, tert-Butyl, and the like). Millifluidic and microfluidic based approaches to peptide synthesis measurably increase the speed of synthesis. GalNAc, neuramidic acid, various sialylated glycan, mannose, sucrose, glucose, fructose, and any OH⁻, COO⁻, NH⁺, NH2⁺ or other similar moiety-containing molecules and their complex derivatives may be assembled as part of on-demand sequences.

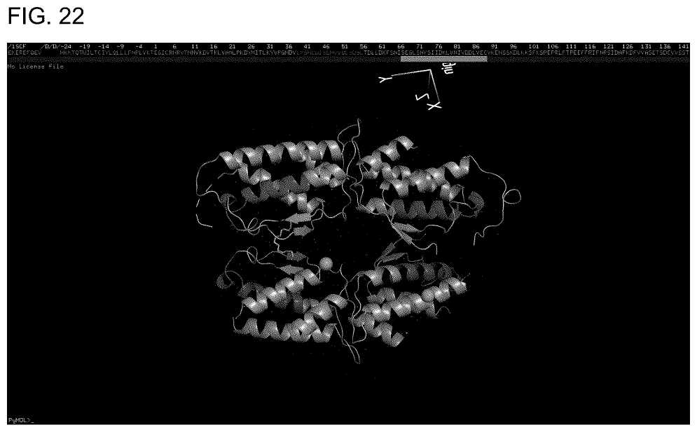

In another aspect, the present Example provides predictive modeling for a nanoparticle seed substrate to target cKit (an SCF fragment). Previous studies report that residues 39-56 of recombinant human SCF (rhSCF) comprise a critical functional region for its ability to enhance expansion of human UCB CD34+ cells (See Shen 2015). This sequence comprises the amino acid sequence LPSHCWISEMVVQLSDSL (PDB ID 1SCF) (SEQ ID NO:21). FIG. 22 depicts a three-dimensional model of a quatrimer of recombinant human SCF with key binding residues highlighted. N-terminal modification with anchor-linkers maintains the outwardly facing binding domain. Here, the anchor-linker example comprises 9R-GGGGSGGGGSLPSHCWISEMVVQLSDSL.

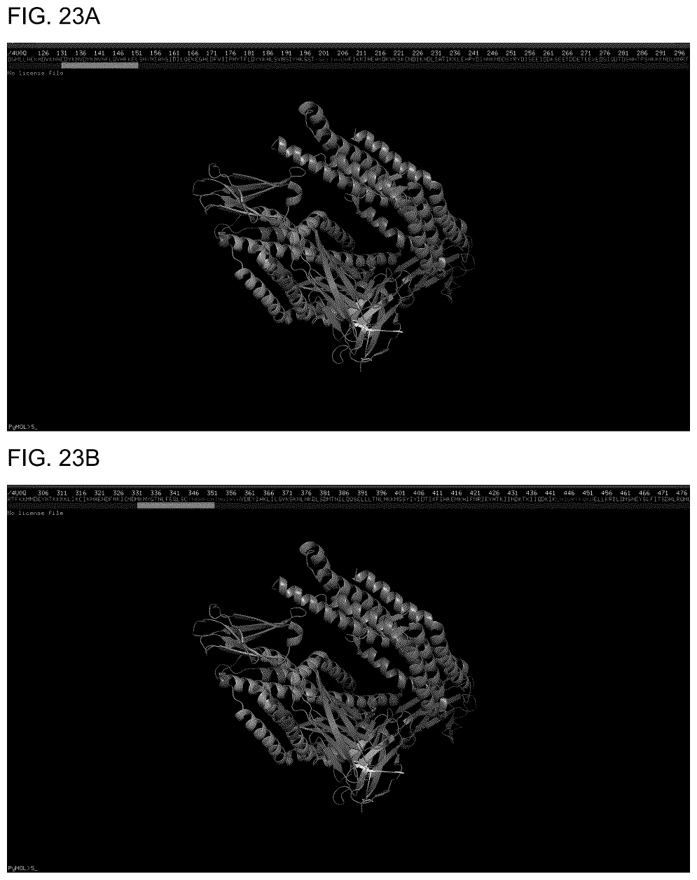

In another aspect, the present Example provides predictive modeling for binding domains of Plasmodium falciparum derived reticulocyte binding protein 5 (PfRH5) including residues 346-357 (YNNNFCNTNGIRYH; SEQ ID NO:22) a disordered loop region, residues 200-208 (YGKYIAVDA; SEQ ID NO:23) a helix region, and residues 444-453 (LNIWRTFQKD; SEQ ID NO:24). FIGS. 23A and 23B depict the binding domains of Plasmodium falciparum derived reticulocyte binding protein 5 (red=binding domains, orange=rest of protein), bound to CD147 (teal) (PDB: ID 4UOQ).

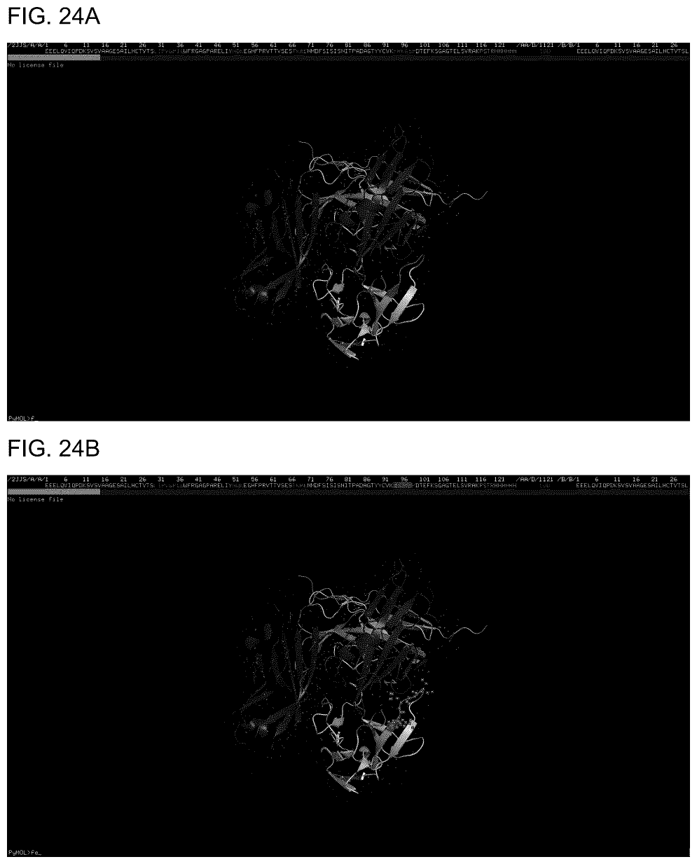

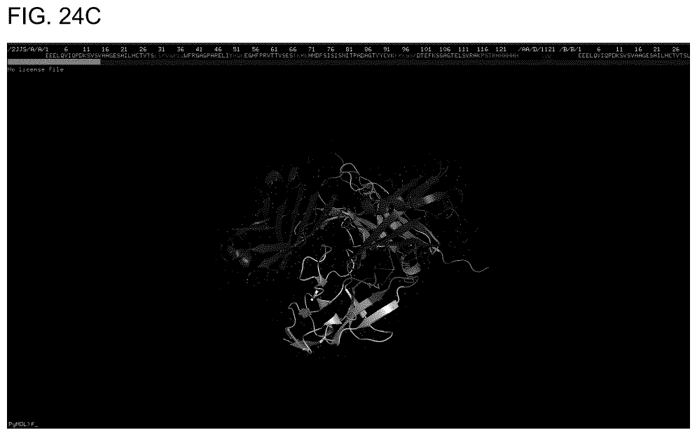

In another aspect, the present Example provides predictive modeling for SIRPa binding with CD47. FIGS. 24A-24C show CD47 (blue) and SIRPa (salmon). Strong (red) and weak (magenta) binding domains are also shown. Key binding sequences include LIPVGP(IQ) (where IQ is optional) (SEQ ID NO:25), NQK, (FR)KGSP (where FR is optional) (SEQ ID NO:26), and TKRE (SEQ ID NO:27). Exemplary anchor-ligand combinations include: anchor-LIPVGPIGGFRKGSP (SEQ ID NO:28); anchor-LIPVGPIGGNQK (SEQ ID NO:29); anchor-LIPVGPIQWFRGAGPARELIYNQK (SEQ ID NO:30); anchor-NQKEGHFPRVTTVSESTKRE (SEQ ID NO:31); anchor-LIPVGPIGGNQKGGGGSTKRE (SEQ ID NO:32); and anchor-LIPVGPIQWFRGAGPARELIYNQKEGHFPRVTTVSESTKRE (SEQ ID NO:33). While the LIPVGPIQ (SEQ ID NO:25) sequence is relatively far away from FRKGSP (SEQ ID NO:26), Applicant predicts that LIPVGPGGFRKGSP (SEQ ID NO:34) should recreate a divalent binding pocket with enhanced avidity, eliminating unnecessary sequences from the protein's tertiary structure. As an alternative, anchor-(linker)-LIPVGPIGGNQKGGGGSTKRE (SEQ ID NO:32) may be used. Here, the extra glycine creates spacing to allow for “compression” of the sequence.

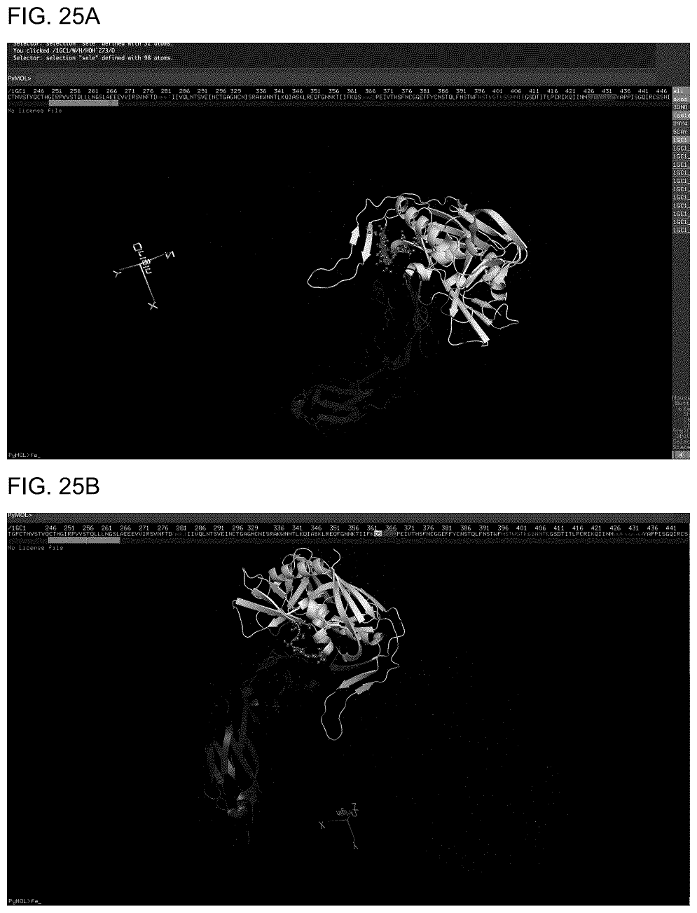

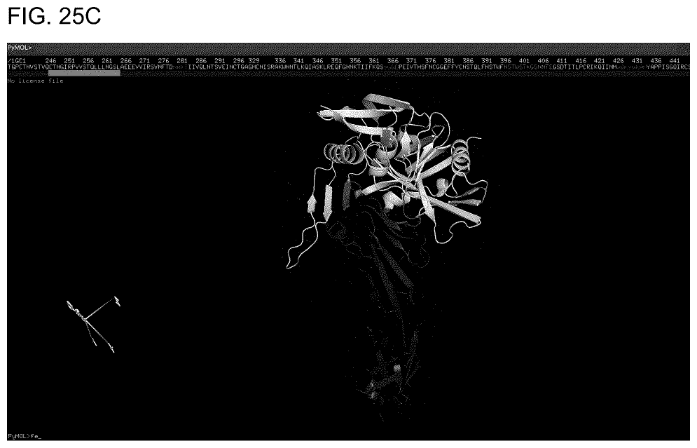

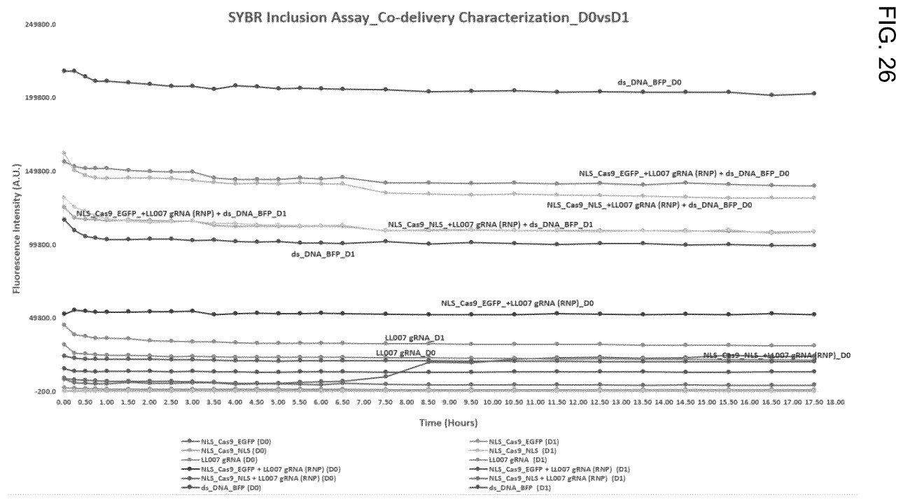

In another aspect, the present Example provides for modeling of CD4 binding to gp120 (from HIV in this Example). FIGS. 25A-25C show CD4 (blue) and HIV gp120 (yellow) along with key binding domains of gp120 for binding to CD4 (in red). Based on these models, sequences NAKT (SEQ ID NO:35), SGGD (SEQ ID NO:36), and WQKVGKAM (SEQ ID NO:37) are predicted to be good binding partners for targeting CD4. Predictive modeling suggests that the SGGD sequence (SEQ ID NO:36) may be coupled directly to an anchor. Further, modeling suggests that the T in the NAKT (SEQ ID NO:35) sequence may be optional for binding. A preferred embodiment may include the sequence RSVNFTDNAKT (SEQ ID NO:38) with an anchor extension on the N-terminus of the sequence. Finally, modeling indicates that the QKV seems may be the most critical residues in the WQKVGKAM (SEQ ID NO:37) sequence to facilitate binding.

### Example 5—Ribonucleoprotein Particle Crosslinking with and without dsDNA

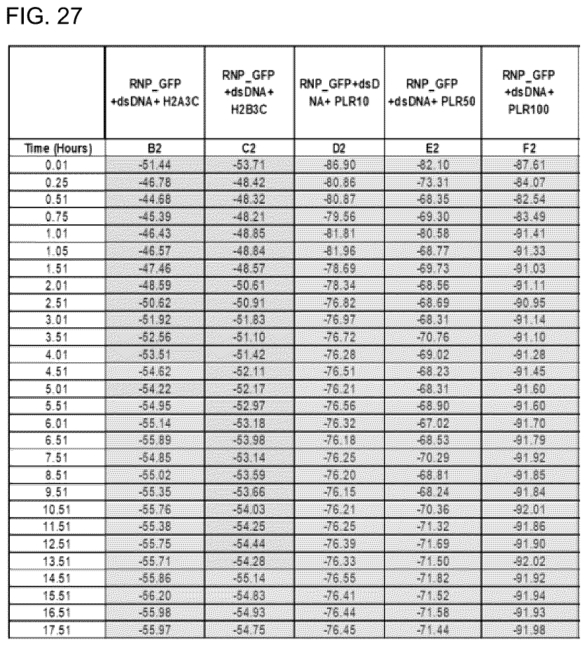

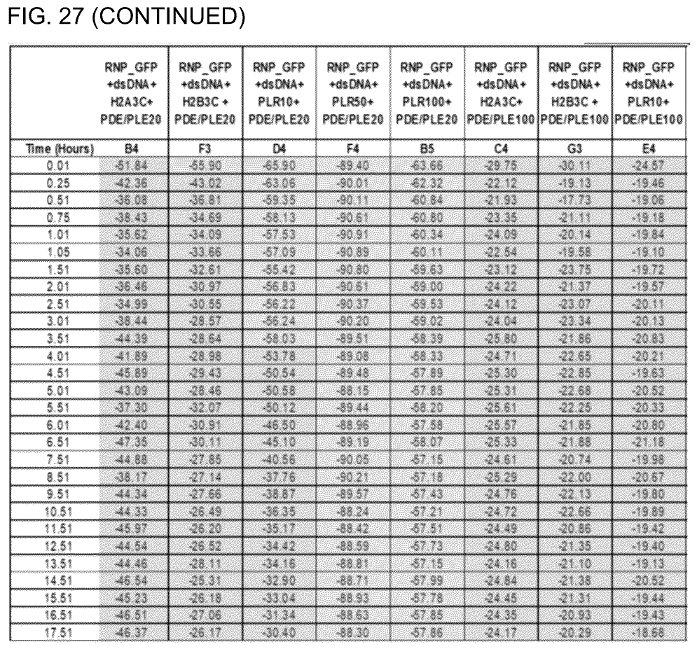

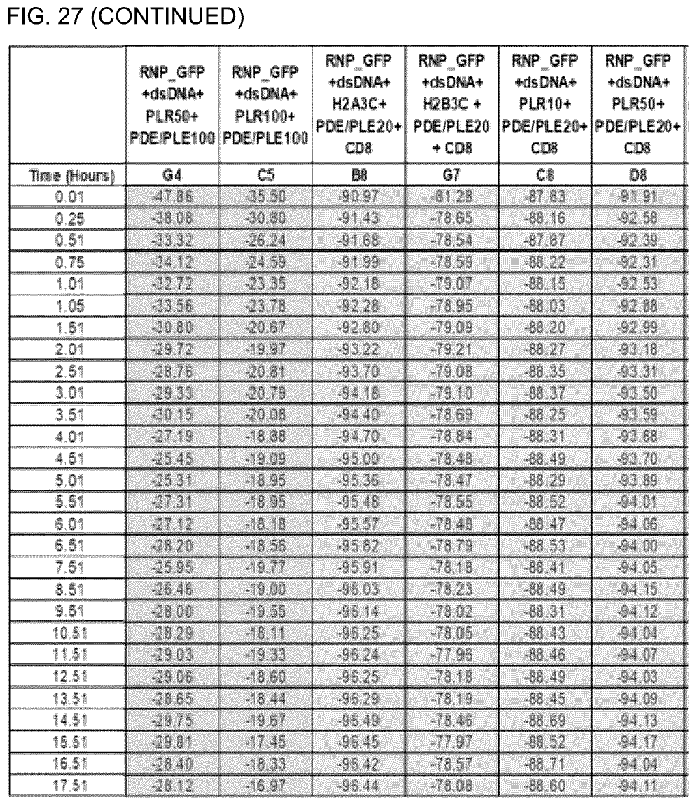

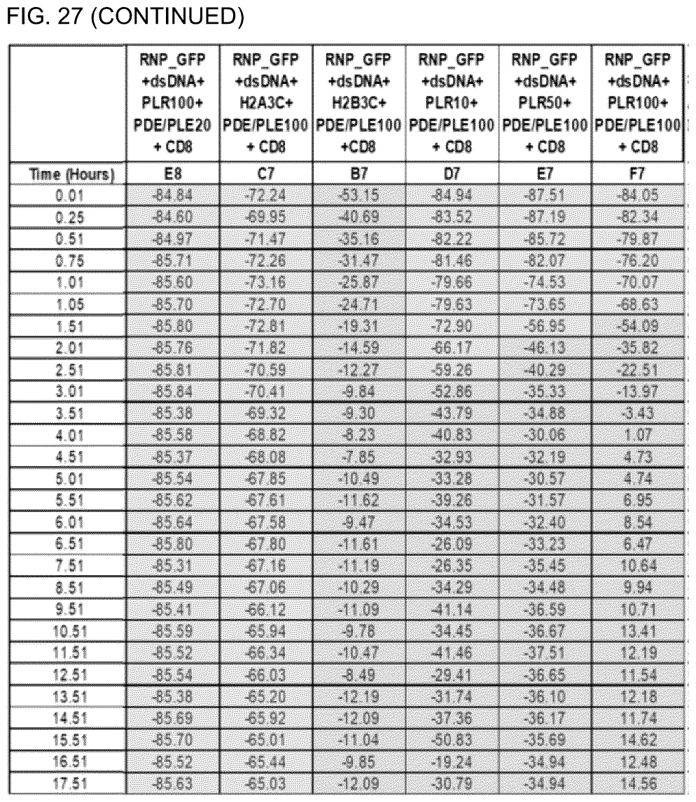

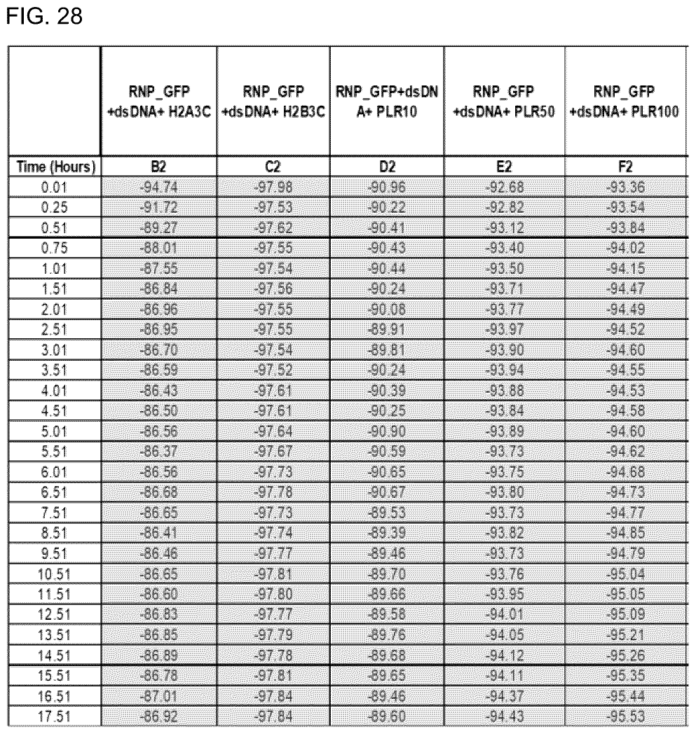

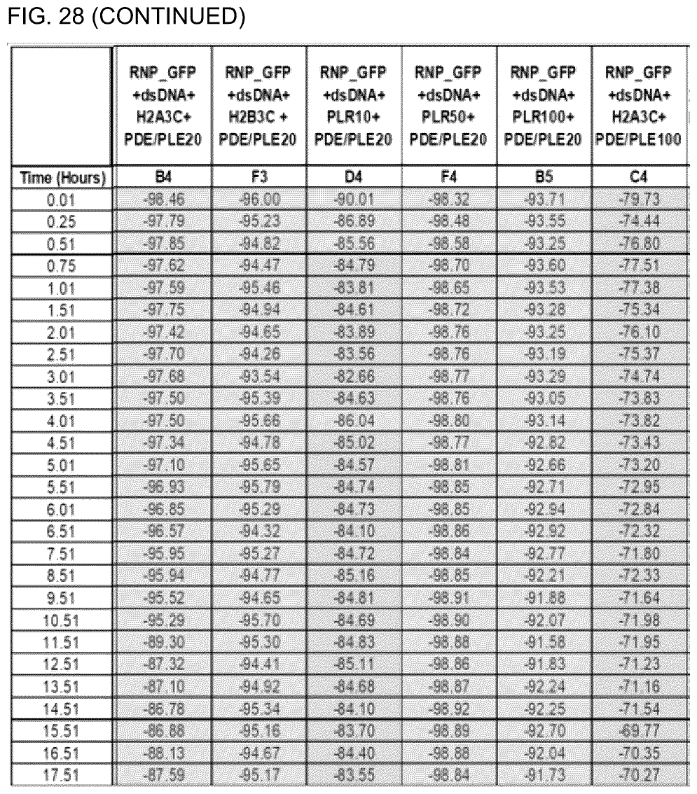

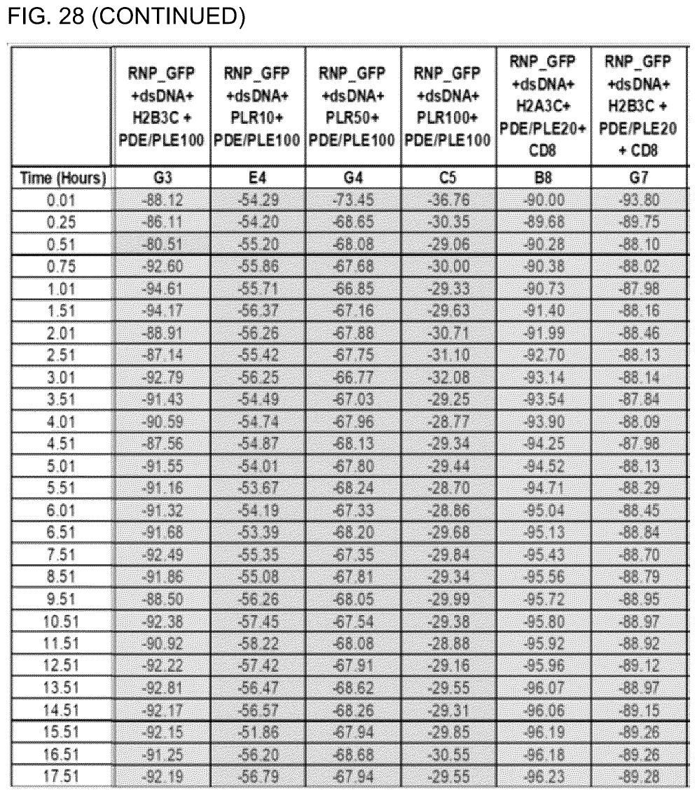

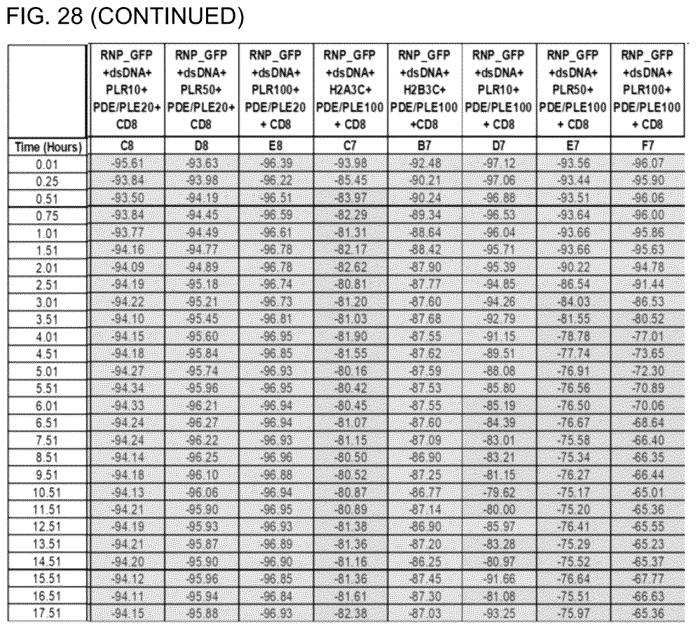

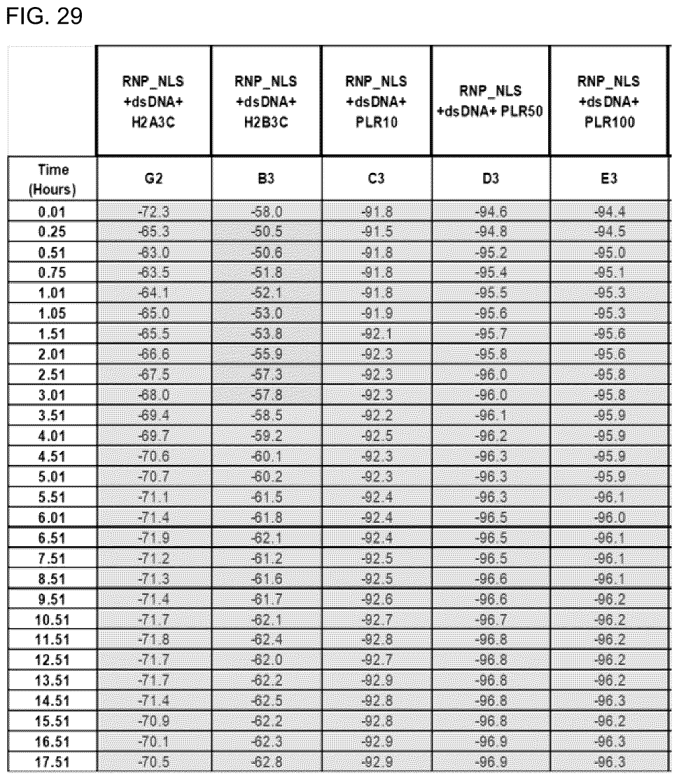

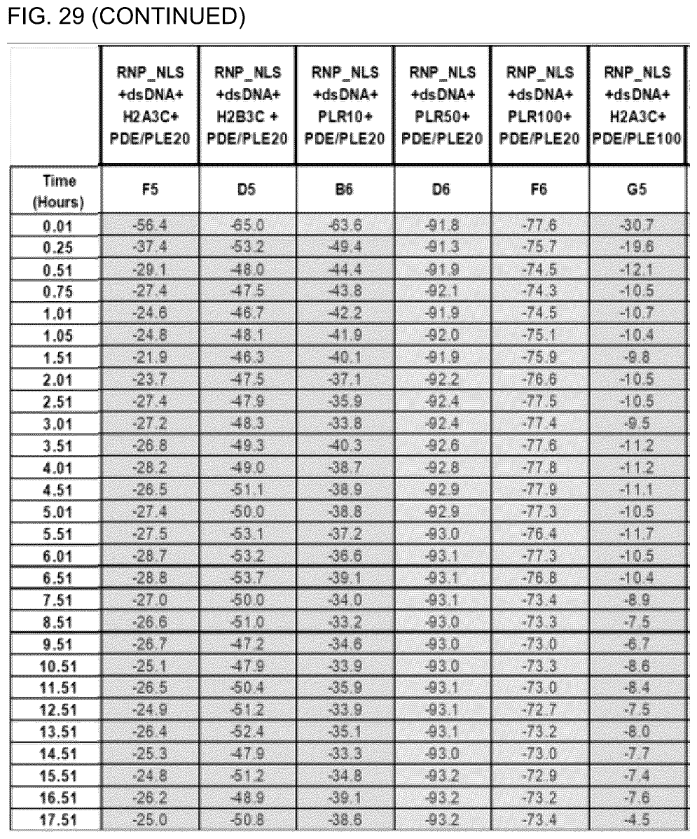

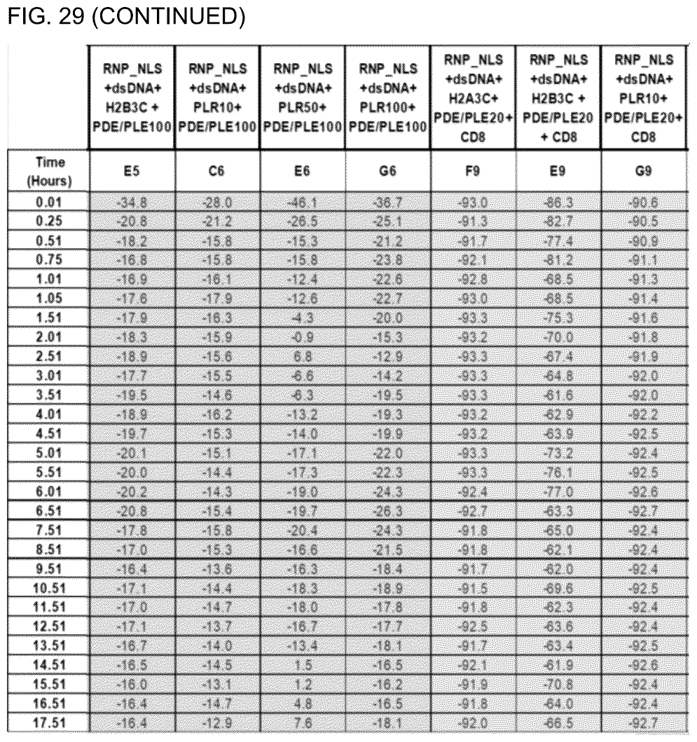

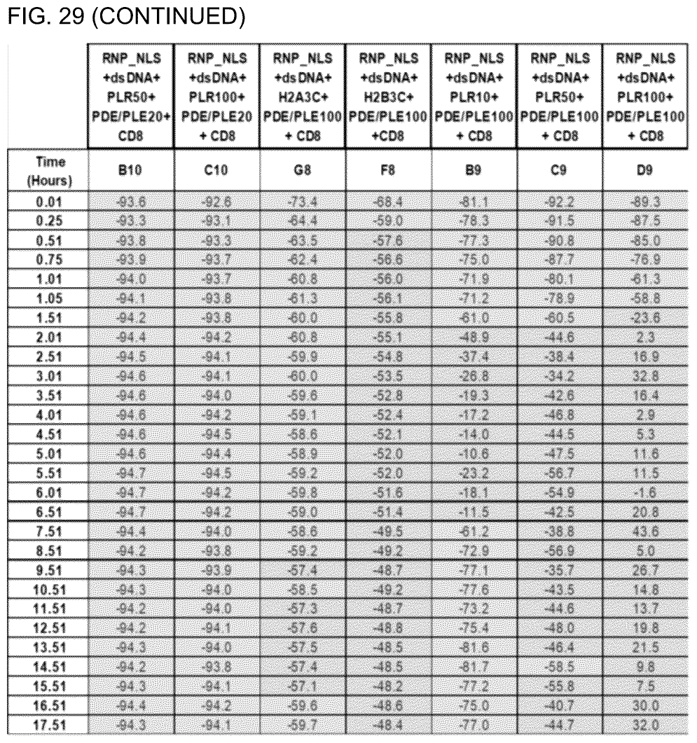

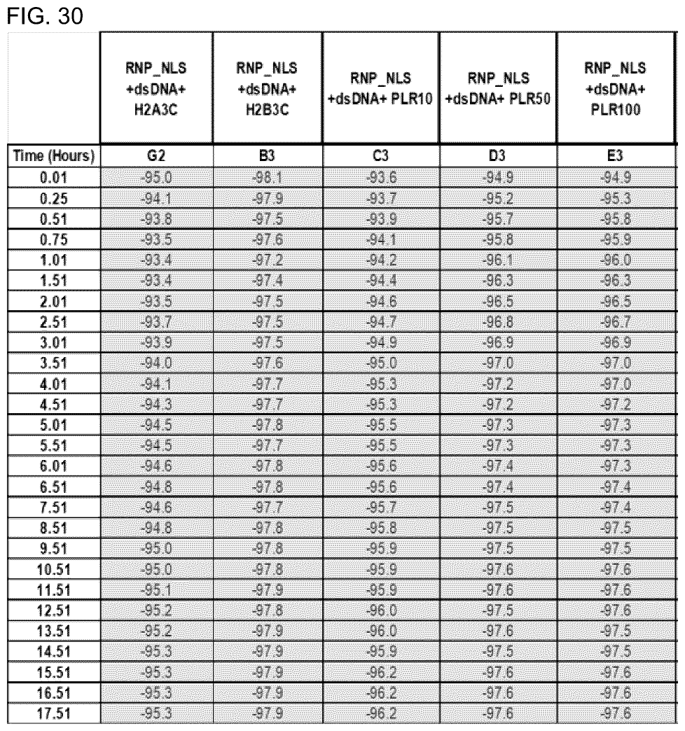

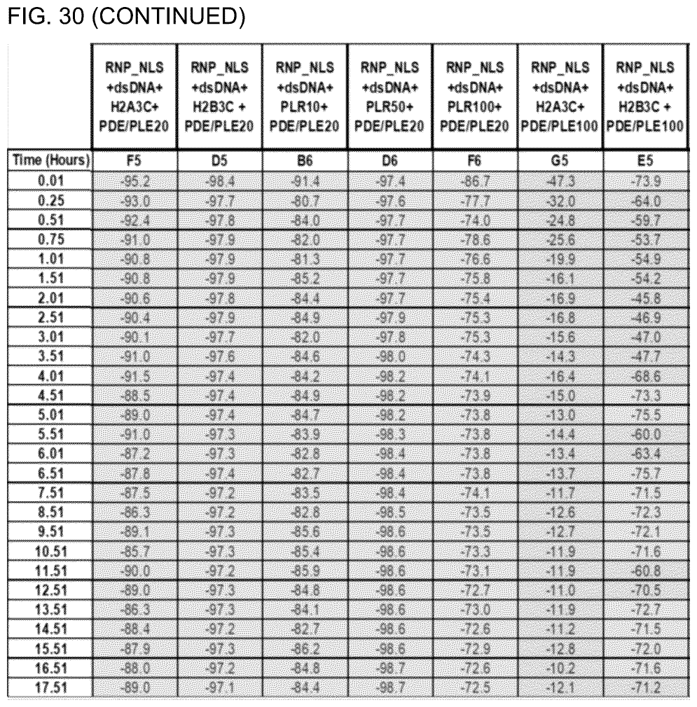

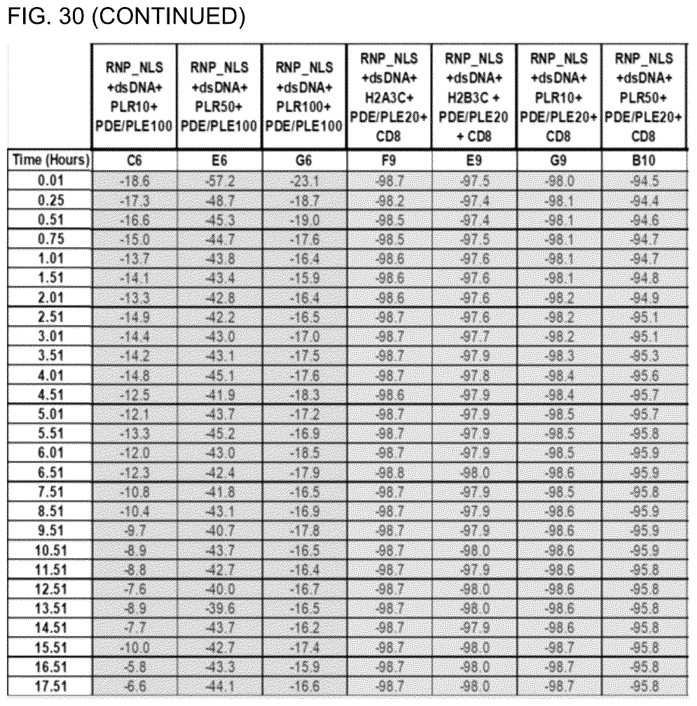

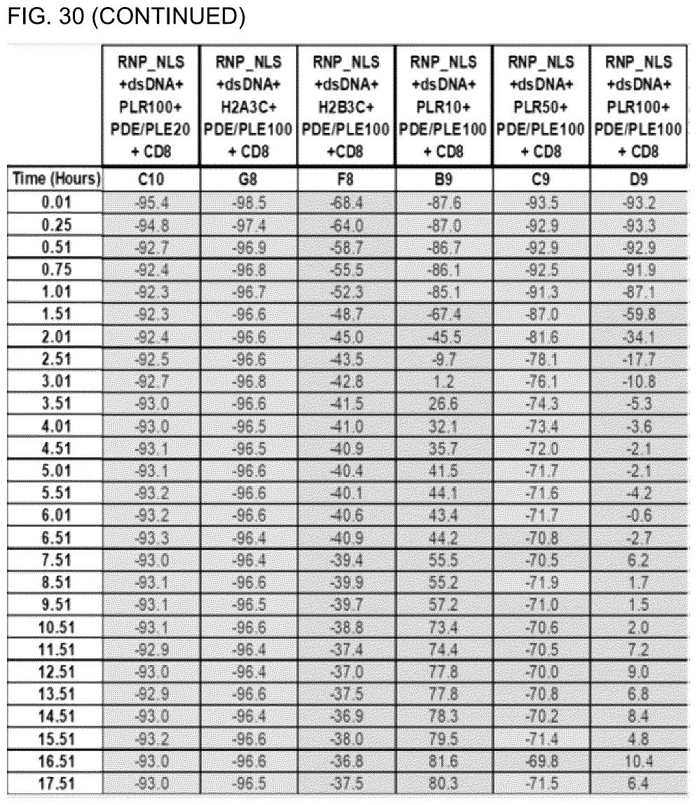

Ribonucleoprotein particle (RNP) crosslinking was investigated with double stranded DNA (dsDNA) and without dsDNA using a SYBR inclusion assay. See FIG. 26. The data from these experiments is quantified in FIGS. 27-30. At day zero these data indicate that groups including poly(L-arginine) (PLR) exhibit better condensation of the nanoparticle core compared to those that include Histone groups. Further, where n=50 (PLR50), PLR appears to perform better than when n=10 or n=100 (PLR10 and PLR100, respectively). Additionally, at day zero, complexes that include PLR and poly(D-glutamic acid) (PDE) where n=20 (PLE20) outperform complexes that include PLRs in combination with PDE/PLE100. In the present Example, all samples with PDE/PLE100 exhibited poor condensation at day zero. The day zero data also reveal that histones combined with PDE/PLE20 and CD8 do a better job of condensing the nanoparticle core than nanoparticle core components combined with histones alone. The data reveal that a histone H2A fragment with three cystine substitutions (H2A3C) performs better than a histone H2B fragment with three cystine substitutions (H2B3C).

During day one of the SYBR inclusion assays, all groups including PDE/PLE100 continued to exhibit poor condensation. In assays including nanoparticle core components combined with H2A3C or nanoparticle core components combined with H2B3C showed less fluorescence on day one (D1) compared to day zero (D0), indicating better condensation. This result may indicate that cross-linking of these components requires longer incubations to stabilize the core. Overall, the day one data indicate that samples including nanoparticle core components combined with H2A3C, PDE/PLE20, and CD8 exhibit marginally better condensation than samples including nanoparticle core components combined with H2B3C, PDE/PLE20, and CD8. Finally, these data indicate that sample groups that include nanoparticle core components combined with PLR10/50/100, PDE/PLE20, and CD8 are comparable to sample groups that include nanoparticle core components combined with H2A3C, PDE/PLE20, and CD8.

### Example 6—Predictive Modeling of Rad51-Cas9 Fusion

Predicative modeling techniques according to the present disclosure may be used to visualize self-assemblable nanoparticle components. In this Example, these predictive modeling techniques have been used to visualize a Rad51-Cas9 fusion protein that may be used as a nanoparticle seed substrate for self-assimilable nanoparticles according to the present disclosure. By modifying key residues as discussed below, it is possible to manipulate the charge of surface-exposed charge patches of the self-assemblies nanoparticle for use as discussed herein.

Previous studies show that Rad51 polymerizes on ssDNA or dsDNA (See Paoletti 2019). Accordingly, using a Cas-Rad51 fusion according to embodiments disclosed herein require a plurality of Rad51 molecules to bind to a DNA to be delivered via a self-assimilable nanoparticle. When there are a minimum of three Rad51 molecules, cells may polymerize the remaining ssDNA/dsDNA molecules intercellularly once delivered. Sequences used in the present Example are presented in Table 4 below.

TABLE 4

| Name/Description | Sequence |
|---|---|
| H2AX catalytic domain for DNA repair | KKATQA(SEP)QEY (133-144) (SEQ ID NO: 39) |
| (may be presented at C-terminus of | |
| Cas9 or nuclease) | |
| Rad51 (PDB ID 5H1B) | MAMQMQLEANADTSVEEESFGPQPISRLEQC |
| | GINANDVKKLEEAGFHTVEAVAYAPKKELINIKG |
| | ISEAKADKILAEAAKLVPMGFTTATEFHQRRSEII |
| | QITTGSKELDKLLQGGIETGSITEMFGEFRTGKT |
| | QICHTLAVTCQLPIDRGGGEGKAMYIDTEGTFR |
| | PERLLAVAERYGLSGSDVLDNVAYARAFNTDH |
| | QTQLLYQASAMMVESRYALLIVDSATALYRTDY |
| | SGRGELSARQMHLARFLRMLLRLADEFGVAVV |
| | ITNQVVAQVDGAAMFAADPKKPIGGNIIAHASTT |
| | RLYLRKGRGETRICKIYDSPCLPEAEAMFAINAD |
| | GVGDAKD (SEQ ID NO: 40) |
| WtSpCas9 (PDB ID 5FQ5) | MDKKYSIGLDIGTNSVGWAVITDEYKVPSKKFK |
| (bold/underlined residues are key | VLGNTDRHSIKKNLIGALLFDSGETAEATRLKRT |
| residues that contribute to wildtype | ARRRYTRRKNRICYLQEIFSNEMAKVDDSFFHR |
| D10A-H840A Cas9 surface-exposed | LEESFLVEEDKKHERHPIFGNIVDEVAYHEKYP |
| charge patches are cationic and/or | TIYHLRKKLVDSTDKADLRLIYLALAHMIKFRGH |
| good spots for mutating to anionic | FLIEGDLNPDNSDVDKLFIQLVQTYNQLFEENPI |
| Glu or Asp) | NASGVDAKAILSARLSKSRRLENLIAQLPGEKK |
| | NGLFGNLIALSLGLTPNFKSNFDLAEDAKLQLSK |
| | DTYDDDLDNLLAQIGDQYADLFLAAKNLSDAILL |
| | SDILRVNTEITKAPLSASMIKRYDEHHQDLTLLK |
| | ALVRQQLPEKYKEIFFDQSKNGYAGYIDGGAS |
| | QEEFYKFIKPILEKMDGTEELLVKLNREDLLRKQ |
| | RTFDNGSIPHQIHLGELHAILRRQEDFYPFLKDN |
| | REKIEKILTFRIPYYVGPLARGNSRFAWMTRKS |
| | EETITPWNFEEVVDKGASAQSFIERMTNFDKNL |
| | PNEKVLPKHSLLYEYFTVYNELTKVKYVTEGMR |
| | KPAFLSGEQKKAIVDLLFKTNRKVTVKQLKEDY |
| | FKKIECFDSVEISGVEDRFNASLGTYHDLLKIIKD |
| | KDFLDNEENEDILEDIVLTLTLFEDREMIEERLKT |
| | YAHLFDDKVMKQLKRRRYTGWGRLSRKLINGI |
| | RDKQSGKTILDFLKSDGFANRNFMQLIHDDSLT |
| | FKEDIQKAQVSGQGDSLHEHIANLAGSPAIKKGI |
| | LQTVKVVDELVKVMGRHKPENIVIEMARENQTT |
| | QKGQKNSRERMKRIEEGIKELGSQILKEHPVEN |
| | TQLQNEKLYLYYLQNGRDMYVDQELDINRLSD |
| | YDVDHIVPQSFLKDDSIDNKVLTRSDKNRGKSD |
| | NVPSEEVVKKMKNYWRQLLNAKLITQRKFDNL |
| | TKAERGGLSELDKAGFIKRQLVETRQITKHVAQI |
| | LDSRMNTKYDENDKLIREVKVITLKSKLVSDFRK |
| | DFQFYKVREINNYHHAHDAYLNAVVGTALIKKY |
| | PKLESEFVYGDYKVYDVRKMIAKSEQEIGKATA |
| | KYFFYSNIMNFFKTEITLANGEIRKRPLIETNGET |
| | GEIVWDKGRDFATVRKVLSMPQVNIVKKTEVQ |
| | TGGFSKESILPKRNSDKLIARKKDWDPKKYGGF |
| | DSPTVAYSVLVVAKVEKGKSKKLKSVKELLGITI |
| | MERSSFEKNPIDFLEAKGYKEVKKDLIIKLPKYS |
| | LFELENGRKRMLASAGELQKGNELALPSKYVN |
| | FLYLASHYEKLKGSPEDNEQKQLFVEQHKHYL |
| | DEIIEQISEFSKRVILADANLDKVLSAYNKHRDK |
| | PIREQAENIIHLFTLTNLGAPAAFKYFDTTIDRKR |
| | YTSTKEVLDATLIHQSITGLYETRIDLSQLGGD |
| | (SEQ ID NO: 41) |
| SpCas9 mutant | MDKKYSIGLDIGTNSVGWAVITDEYDVPSDDFK |
| (bolded/underlined residues indicate | VLGNTDEHSIDDDLIGALLFDSGETAEATRLKRT |
| mutant sites on surface versus WT | ARRRYTRRKNRICYLQEIFSNEMAKVDDSFFHR |
| for electrostatic surface modulation) | LEESFLVEEDKKHERHPIFGNIVDEVAYHEKYP |
| | TIYHLRKKLVDSTDKADLRLIYLDLAHMIKFRGH |
| | FLIEGDLNPDNSDVDDLFIELVETYNELFEENPI |
| | NADDVDAKAILSARLSKSRRLENLIAQLPGEDD |
| | NGLFGNGIALSLGLTPNFDSNFDLAEDAKLQLS |
| | KDTYDDDLDNLLDQIGDQYADLFLAAKNLSDAIL |
| | LSDILEVNTEETDAPLSASMIKRYDEHHQDLTLL |
| | KALVRQQLPEKYKEIFFDQSKNGYAGYIDGGAS |
| | QEEFYKFIDPILEDMDGTEELLVDLNREDLLRK |
| | QRTFDNGSIPHQIHLGELEAILEEQEDFYPFLKD |
| | NEEKIEKILTFRIPYYVGPLARGNSRFAWMTRK |
| | SEETITPWNFEEVVDKGASAQSFIERMTNFDKN |
| | LPNEKVLPKHSLLYEYFTVYNELTKVDYVTEGM |
| | EDPAFLSGEQKKAIVDLLFKTNRKVTVKQLKED |
| | YFKKIECFDSVEISGVEDRFNASLGTYEDLLKIID |
| | DDDFLDNEENEDIGEDIVLTLTLFEDREMIEERL |
| | KTYAHLFDDDVMKQLDRREYTGWGRLSRKLIN |
| | GIRDKQSGKTILDFLKSDGFANRNFMQLIHDDS |
| | LTFKEDIQKAQVSGQGDSLHEHIANLAGSPAIK |
| | KGILQTVKVVDELVKVMGRHKPENIVIEMAREN |
| | QTTQKGQKNSRERMKRIEEGIKELGSQILKEHP |
| | VENTQLQNEKLYLYYLQNGEDMYVDQELDINE |
| | LSDYDVDHIVPQSFLKDDSIDNKVLTRSDKNRG |
| | KSDNVPDEEVVKKMKNYWRQLLNAKLITQRKF |
| | DNLTKAERGGLSELDKAGFIKRQLVETRQITKH |
| | VAQILDSRMNTKYDENDKLIREVKVITLKSKLVS |
| | DFRKDFQFYKVREINNYHHAHDAYLNAVVGTA |
| | LIKKYPKLESEFVYGDYKVYDVRKMIAKSEQEIG |
| | KATAKYFFYSNIMNFFKTEITLANGEIRKRPLIET |
| | NGETGEIVWDKGRDFATVRKVLSMPQVNIVKK |
| | TEVQTGGFSKESILPKRNSDKLIARKKDWDPKK |
| | YGGFDSPTVAYSVLVVAKVEKGKSKKLKSVKEL |
| | LGITIMERSSFEKNPIDFLEAKGYKEVKKDLIIKL |
| | PKYSLFELENGRKRMLASAGELQKGNELALPS |
| | KYVNFLYLASHYEKLKGSPEDNEQKQLFVEQH |
| | KHYLDEIIEQISEFSKRVILADANLDKVLSAYNKH |
| | RDKPIREQAENIIHLFTLTNLGAPAAFKYFDTTID |
| | RKRYTSTKEVLDATLIHQSITGLYETRIDLSQLG |
| | GD (SEQ ID NO: 42) |

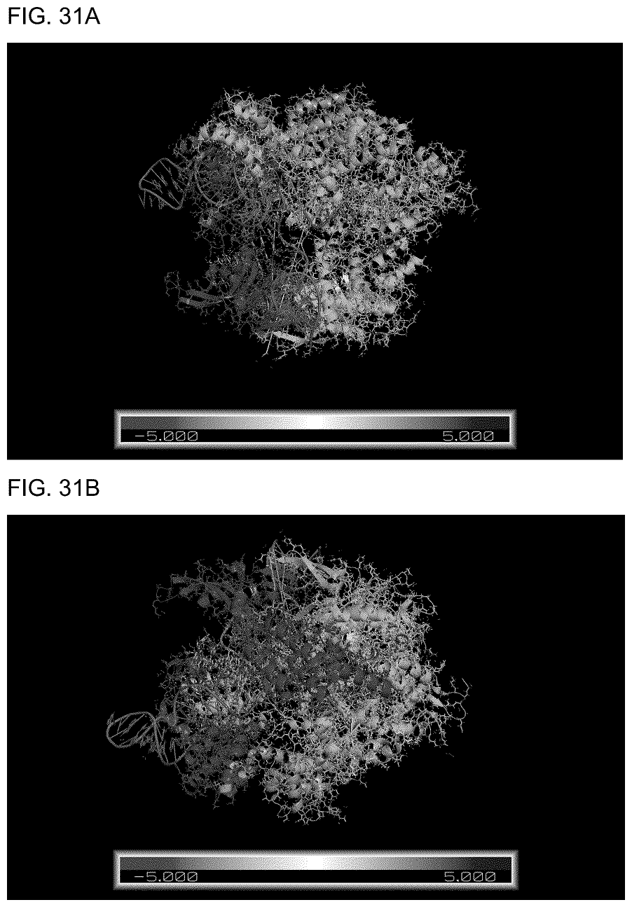

Key residues for surface charge manipulation are identified in Table 4 above (bolded and underlined) and in FIGS. 31A-31B (green). These key residues are contributors to wildtype D10A-H840A Cas9 surface-exposed charge patches and are cationic and/or good spots for mutating to anionic Glu or Asp). According to the present Example, the following substitutions may be made for charge manipulation: Substitute all K for D, and all R for E. Substitute all H for E. Substitute all Q for E. Substitute all N for D. Substitute all I for E and L for D. Substitute all G, A and S for D. Modeling indicates that R859 and K866 are potentially good mutagenic sites, but sit too close to A840, N854 and N863 which either make Cas9 into a nickase (H840A, N863A) or reduce activity (N854). Accordingly, in the present Example these residues remain unmodified and are colored in wheat in the three-dimensional model of FIGS. 31A-31B. L241G and L616G mutation sites (blue) are also shown, per immunogenicity minimizing motifs as identified previously. See FIGS. 31A-31B; Ferdosi 2018; Ferdosi 2019. Therefore, the following residues are key residues for charge manipulation: K26D, K30D, K31 D, R40E, K44D, K45D, N46D, A157D, K183D, Q187E, Q190E, Q194E, S204D, G205D, K233D, K234D, L241G, K253D, A280D, R307E, 1312E, K314D, K377D, K382D, K392D, H420E, R424E, R425E, R437E, K528D, R535E, K536D, H595E, K602D, K604D, L616G, K645D, K652D, R655E, R832E, S872D. Optional residues for modification include: R859E, K866D, K960D. Table 5 (below) details mutagenesis at specific residues and predicted resultant modification.

TABLE 5

| Mutagenesis | |
|---|---|
| residue | Description |
| 10 | D→A: Target DNA noncomplementary to the crRNA is not cleaved; |
| | nickase activity. Processes guide RNAs. In vivo, loss of Cas9- |
| | mediated CRISPR interference in plasmid transformation. Able to |
| | bind guide RNAs and target DNA but not cleave DNA; when |
| | associated with A-840. |
| 15 | S→A: Decreased DNA cleavage. |
| 66 | R→A: Significantly decreased DNA cleavage. |
| 70 | R→A: No DNA cleavage. |
| 74 | R→A: Significantly decreased DNA cleavage. |
| 78 | R→A: Moderately decreased DNA cleavage |
| 97-150 | Missing: no nuclease activity. |
| 165 | R→A: Moderately decreased DNA cleavage. |
| 175-307 | Missing: About 50% nuclease activity. |
| 312-409 | Missing: No nuclease activity. |
| 475-477 | PWN→AAA: Slight decrease in target DNA cleavage and DNA- |
| | binding. Almost complete loss of DNA cleavage and binding; when |
| | associated with 1125-A--A-1127. |
| 762 | E→A: Only cleaves one DNA strand, probably the noncomplementary |
| | strand. Processes guide RNAs correctly. In vivo, loss of Cas9- |
| | mediated CRISPR interference in plasmid transformation. |
| 840 | H→A: Target DNA complementary to the crRNA is not cleaved; |
| | nickase activity. In vivo, loss of Cas9-mediated CRISPR interference |
| | in plasmid transformation. Able to process and bind guide RNAs and |
| | target DNA but not cleave DNA; when associated with A-10. |
| 854 | N→A: Decreased DNA cleavage. Processes guide RNAs correctly. |
| | In vivo, retains Cas9-mediated CRISPR interference in plasmid |
| | transformation. |
| 863 | N→A: Only cleaves one DNA strand, probably the complementary |
| | strand. Processes guide RNAs correctly. In vivo, loss of Cas9- |
| | mediated CRISPR interference in plasmid transformation. |
| 982-983 | HH→AA: Processes guide RNAs correctly. |
| 982 | H→A: Decreased DNA cleavage. In vivo, loss of Cas9-mediated |
| | CRISPR interference in plasmid transformation. |
| 983 | H→A: Only cleaves one DNA strand, probably the noncomplementary |
| | strand. |
| 986 | D→A: Only cleaves one DNA strand, probably the noncomplementary |
| | strand. Processes guide RNAs correctly. In vivo, loss of Cas9- |
| | mediated CRISPR interference in plasmid transformation. |
| 1099-1368 | Missing: No nuclease activity. |
| 1125-1127 | DWD→AAA: No change in target DNA cleavage, slight decrease in |
| | DNA-binding. Almost complete loss of DNA cleavage and binding; |
| | when associated with 475-A--A-477. |
| 1132 | G→C: Probably inactivates protein. |
| 1333-1335 | RKR→AKA: Nearly complete loss of target DNA cleavage. |
| 1333 | R→A: Dramatically reduced target DNA binding, slightly decreased |
| | target cleavage. |
| 1335 | R→A: Dramatically reduced target DNA binding, slightly decreased |
| | target cleavage. |

### Example 7—Simulating Binding Pockets to the Amino Acid Level Using AG Values

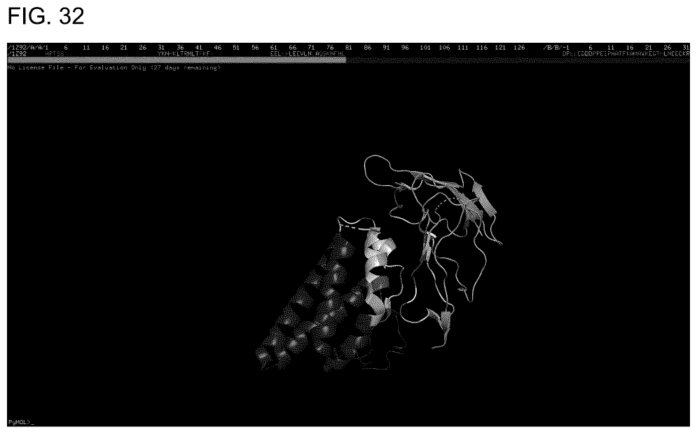

Three-dimensional modeling according to the present disclosure can be used to predict binding to the individual amino acid level using AG values. In the present Example, IL2-IL2RA binding (PDB ID 1Z92) as determined by contact mapping via PDBePISA is modeled. See FIG. 32. Green residues increase binding affinity and have AG values of <−0.10 are colored green, while AG values of −0.10-0.10 are colored pale green. Red residues decrease binding affinity and have AG values >0.10. Contiguous stretches of pale green residues containing green residues may be synthesized using peptide synthesis robotics, using various optional anchor-linker pairings. Red residues may also be mutated to change AG values to <0.

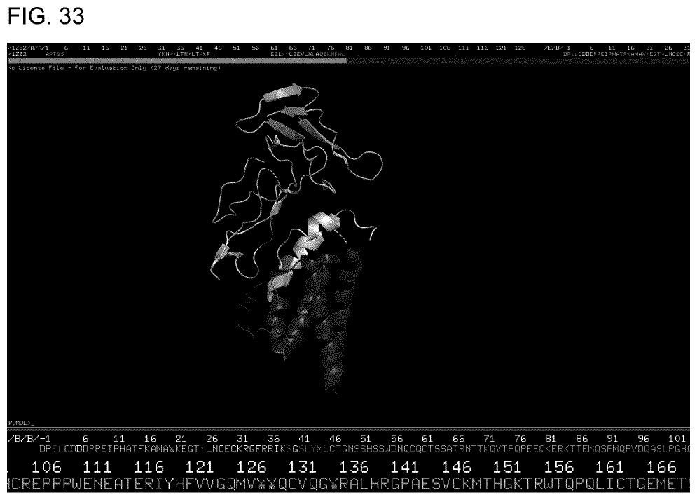

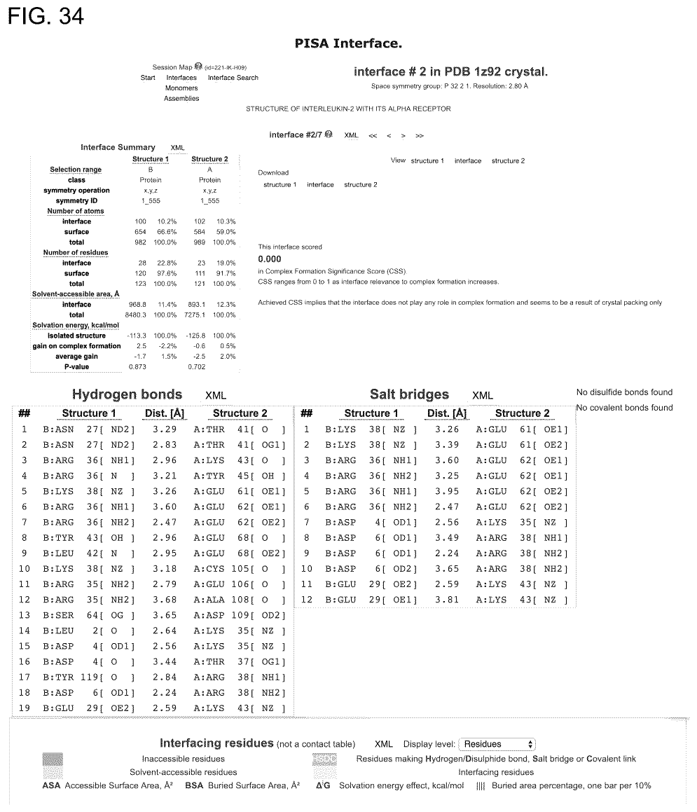

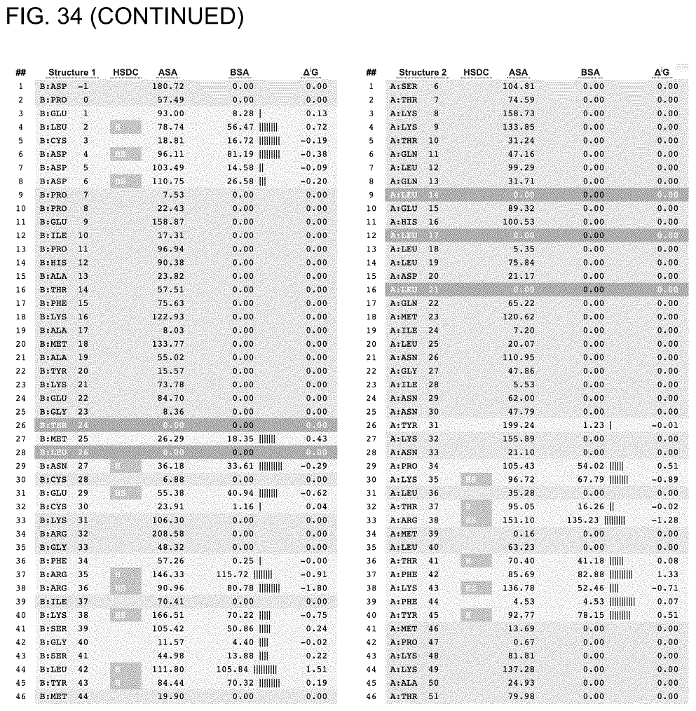

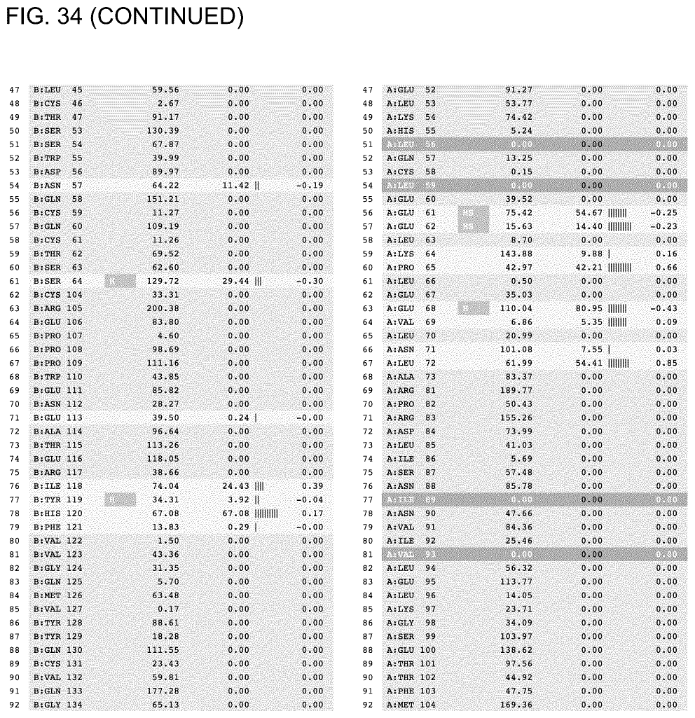

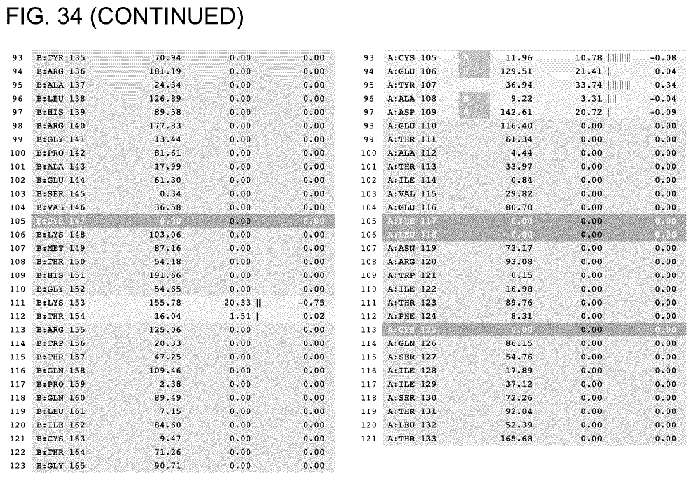

In the present Example, the sequences YKNPKLTRMLTFKFY [31-45] (SEQ ID NO-7) and EELKPLEEVLNLA [61-73] (SEQ ID NO-8) may be synthesized as anchor-linker-ligand, ligand-linker-anchor, anchor-ligand, ligand-anchor, or standalone ligand variants to generate affinity for the cellular receptor. A machine learning approach can optimize truncated variants to have optimal binding kinetics, either as wildtype or modified forms. For example, YKN(X)KLTRMLT(X)KF [31-44] (Y45 truncated; SEQ ID NO:43) and EEL(X)(X)LEEVLN(X)A [61-73] (SEQ ID NO:44) are ideal sequences. The largest contiguous stretch of amino acids with net global minimum summed ΔG values comprises an “ideal sequence.” Here, Y45 is truncated since it contributes +ΔG to the amino acid sequence. Red sequences of a known sequence may be mutated into variants that do not form a repulsive effect with the corresponding red sequences shown in the IL2RA receptor or another wildtype receptor with a known structure. In this example, KP [64-65] of IL2 forms a repulsive effect with S39 and SLY [41-43] of IL2RA. Mutating K64 on IL2 to a hydrophobic amino acid can restore a negative ΔG value, as may mutating P65 into a hydrogen-bond-forming amino acid to enhance binding to serine and tyrosine. This approach may be used to identify optimal contiguous stretches of amino acids for receptor-specific binding with truncated fragments of wildtype proteins, with optional modifications. See FIGS. 33-34. Contacting mapping of the interface of IL2 with IL2RA (PDB ID 1Z92) as determined by PDBePISA. Hydrogen bonds and salt bridges are shown, as well as ΔG values. These ΔG values are used as part of a machine learning approach to color binding affinity generating residues green. Id.

### Example 8—Designing a CD3 or CD28-Targeting Ligand

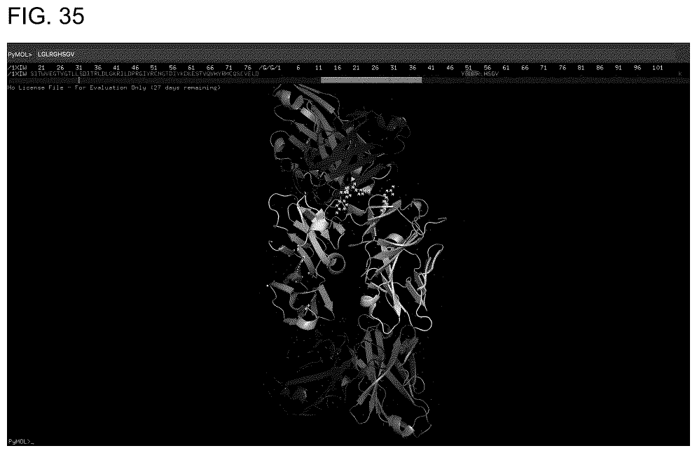

Three-dimensional modeling according to the present disclosure can be used to design ligands that specifically target particular cells or other moieties. For example, in the present Example, a ligand is designed to target CD3. Here, FIG. 35 depicts a three-dimensional model showing the crystal structure of human CD3-e/d dimer in complex with a UCHT1 single-chain antibody fragment (PDB ID: 1XIW). In this model, YTSRLHSGV (SEQ ID NO:45) has the lowest ΔG value of this interaction. This sequence may be changed to (2 hydrophobic+1 glycine/alanine), e.g., LGLRGHSGV (SEQ ID NO:46), LLGRGHSGV (SEQ ID NO:47), GLLRGHSGV (SEQ ID NO:48) to increase affinity. YYT is repulsed by F13 on CD3d. See FIG. 35.

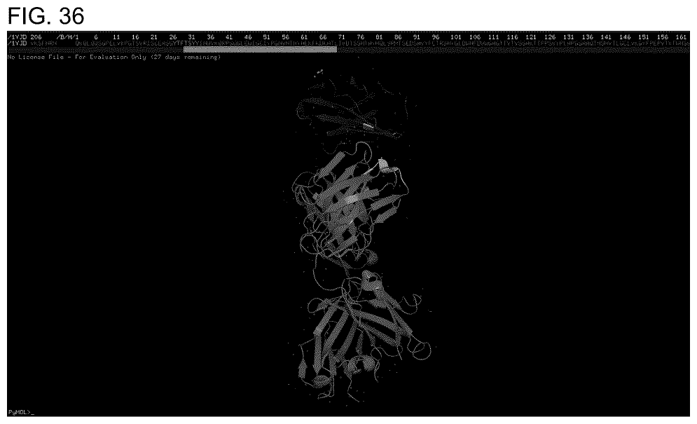

Three-dimensional modeling according to the present disclosure in the present Example is additionally used as proof of concept to design a CD28 targeting ligand. T cells naturally interact with CD80 and CD86 on antigen-presenting cells, which binds to CD28 and causes activation of T cells. See FIG. 36.

Using contact mapping, even in the absence of looking at structural data manually, Applicant determined that the following amino acids are critical for antibody binding to CD28. By synthesizing these sequences with modifications where ΔG>0 if relevant, truncated fragments may exhibit increased affinity for the target marker. This antibody can be compressed to 7 amino acids to generate affinity as predicted by ΔG values between the binding residues on CD28. This antibody can be compressed to 7 amino acids to generate affinity as predicted by ΔG values between the binding residues on CD28. See FIG. 36.

### REFERENCES

1. Clouaire et al. “Comprehensive mapping of histone modifications at DNA double-strand breaks deciphers repair pathway chromatin signatures.” Mol Cell 72(2):250-262 (2018).
2. Ferdosi et al. “Multifunctional CRISPR/Cas9 with engineered immunosilenced human T cell epitopes.” bioRxiv 360198 (2018) (https://www.biorxiv.org/content/biorxiv/early/2018/07/02/360198.full.pdf).
3. Ferdosi et al. “Multifunctional CRISPR-Cas9 with engineered immunosilenced human T cell epitopes.” Nat Commun 10(1):1842 (2019).
4. Lee et al. “Screening of cell-penetrating peptides using mRNA display.” Biotechnol 7(3):387-396 (2012).
5. Paoletti et al. “Molecular flexibility of DNA as a key determinant of RAD51 recruitment,” bioRxiv 392795 (2019) (https://www.biorxiv.org/content/10.1101/392795v2.full).
6. Rodrigues et al. “Uptake and cellular distribution of nucleolar targeting peptides (NrTPs) in different cell types.” Biopolymers 104(2):101-109 (2015).
7. Shen et al. “Residues 39-56 of stem cell factor protein sequence are capable of stimulating the expansion of cord blood CD34+ cells.” PLoS One 10(10):e0141485 (2015).
8. Sofueva et al. “BRCT domain interactions with phospho-histone H2A target Crb2 to chromatin at double-strand breaks and maintain the DNA damage checkpoint.” Mol Cell Biol 30(19):4732-4743 (2010).
9. Vandenberg et al. “Characterization of a type IV collagen major cell binding site with affinity to the alpha 1 beta 1 and the alpha 2 beta 1 integrins.” J Cell Biol 113(6):1475-1483 (1991).
10. Yamano et al. “Crystal structure of Cpf1 in complex with guide RNA and target DNA.” Cell 165(4):949-962 (2016).
11. Yan et al. “Structure basis of yH2AX recognition by human PTIP BRCT5-BRCT6 domains in the DNA damage response pathways.” FEBS Lett 585(24):3874-3879 (2011).

1. A composition, comprising:
a non-proteinaceous nanoparticle seed substrate having a three-dimensional surface comprising a plurality of binding patches; and
a plurality of moieties coupled to the plurality of binding patches via hydrophobic or hydrophilic interactions;
wherein the combination of coupled moieties inhibits nanoparticle seed substrate aggregation by coupling of the nanoparticle seed substrate to a substrate.

2. (canceled)

3. (canceled)

4. The composition of claim 1, wherein the non-proteinaceous nanoparticle seed substrate is selected from an electrostatic, lipidic, gold, or metallic particle.

5. A composition, comprising:
a nanoparticle seed substrate having a zwitterionic three-dimensional charge-tunable surface comprising a plurality of binding patches; and
a plurality of moieties coupled to the plurality of binding patches;
wherein the combination of coupled moieties inhibits nanoparticle seed substrate aggregation by electrostatic coupling of the nanoparticle seed substrate to a substrate.

6. The composition of claim 5, comprising:
the plurality of binding patches comprising a plurality of cationic and a plurality of anionic binding patches; and
the nanoparticle seed substrate having a zwitterionic charge ratio, wherein the zwitterionic charge ratio is defined by the number of the cationic binding patches to the number of anionic binding patches (+/−) and the charge ratio is modified compared to an unmodified seed substrate.

7. The composition of claim 6, further comprising:
at least one moiety comprising a modified protein, the modified protein comprising:
an anchor, wherein the anchor is designed to interact with a binding patch and wherein the interaction between the anchor and the binding patch may be hydrophilic, hydrophobic, electrostatic, covalent, or non-covalent;
at least one linker, wherein at least one linker is coupled to the anchor; and
a payload coupled directly to the anchor and to the at least one linker, or
a functional domain coupled directly to the anchor and to the at least one linker.

8. (canceled)

9. The composition of claim 7, wherein
the plurality of cationic binding patches is electrostatically bound to a plurality of negatively charged moieties; and
the plurality of anionic binding patches is electrostatically bound to a plurality of positively charged moieties.

10. (canceled)

11. (canceled)

12. The composition of claim 9, further comprising:
at least one moiety, wherein the moiety is a fusion polymer comprising:
(i) a polymeric molecule selected from the group consisting of a gRNA molecule, a donor DNA molecule, an mRNA molecule, an siRNA molecule, a dsRNA molecule, an aptamer, a charge-switchable polymer, a bioreducable polymer, a glycosaminoglycan (GAG), a polyethylene glycol (PEG) chain, an N-(2-Hydroxypropyl) methacrylamide (HPMA) chain, an oligosaccharide, a proteoglycan, an anionic peptide sequence, a cationic peptide sequence, a peptide sequence comprising α-aminoisobutyric acid, a cell penetrating peptide (CPP), a β-peptide, a γ-peptide, a δ-peptide, a peptide mimetic, an anionic glycopeptide sequence, a cationic glycopeptide sequence, a peptoid, a peptoid mimetic, a σ-strand peptoid, a peptidomimetic foldamer, a nucleotidomimetic foldamer, an abiotic foldamer, a sphingolipid, sphingosine-1-phosphate, a ceramide, a ganglioside, a lipid, an anionic lipid, a cationic lipid, a lipid derivative, a native lipid, a synthetic lipid, a polymer, an anionic polymer, a cationic polymer, alginate, agmatine, gelatin, a carboxylate-rich polymer, a phosphate-rich polymer, a sulfate-rich polymer, a sugar, a polysaccharide, a multi-branched polysaccharide, a poly(nucleotide), a poly(nucleotide) mimetic, a poly(aspartic acid)-rich sequence, a poly(glutamic acid)-rich sequence, a branched polymer or co-polymer variant thereof, a dendrimeric polymer or co-polymer variant thereof, a p(asp)[DET] molecule, a poly(glycolic acid), a poly(lactic acid), a poly(lactic-co-glycolic acid), an aliphatic chain, an amine-rich polymer, a charge-modified polymeric backbone, an anionic charge-modified polymer backbone, a cationic charge-modified polymer backbone, a poly(β-amino ester), a negatively charge-functionalized poly(β-amino ester), a positively charge-functionalized poly(β-amino ester), a modified amino acid, a modified amino acid mimetic, a lipid, a cationic lipid, an anionic lipid, a histone-derived sequence, an NLS-derived sequence, a subcellular-localizing sequence, a subcellular-functional sequence, a DNA-binding protein, an RNA-binding protein, an anchor-linker-ligand complex, an anchor-ligand complex, an anchor-functional domain complex, an anchor-linker-functional domain complex, a cationic charge-modified polymer backbone or a co-polymer variant thereof, and a multi-domain polymer:
(ii) a stereoisomer of (i); or
(iii) a polymeric molecule comprising an additional moiety as a contiguous portion of its sequence, wherein the additional moiety is selected from the group consisting of a functional domain, a Payload domain, Polyethylene glycol (PEG), N-(2-Hydroxypropyl) methacrylamide (HPMA), poly(sarcosine), a biocompatible linker or terminal polymer sequence assisting in forming phase-separation, a gRNA molecule, a donor DNA molecule, an mRNA molecule, an siRNA molecule, an miRNA molecule, a dsRNA molecule, an aptamer, a glycosaminoglycan (GAG), an oligosaccharide, a proteoglycan, an anionic peptide sequence, an anionic glycopeptide sequence, a sphingolipid, sphingosine-1-phosphate, a ceramide, a ganglioside, an anionic lipid, an anionic polymer, alginate, gelatin, a carboxylate-rich polymer, a phosphate-rich polymer, a sulfate-rich polymer, a peptoid, a negatively charge-functionalized poly(β-amino ester), a polysaccharide, a poly(aspartic acid) rich sequence, a poly(glutamic acid) rich sequence, agmatine, a charge-modified polymer backbone, an anionic charge-modified polymer backbone, a cationic charge-modified polymer backbone, a branched polymer backbone or a co-polymer variant thereof, a dendrimeric polymer backbone or a co-polymer variant thereof, and a charge-switchable polymer.

13.-15. (canceled)

16. The composition of claim 12, wherein the multi-domain polymer comprises a plurality of domains selected from cationic domains, anionic domains, and neutral domains.

17. The composition of claim 12, wherein the functional domain comprises one or more of the following: a ligand, an endosomolytic domain, a subcellular functional domain, a subcellular trafficking domain, a histone-mimetic domain, a nuclear-material-mimetic domain, an environmental-specific unpackaging domain, a protein-corona-inhibitory domain, a macrophage-endocytosis-inhibitory domain, a receptor agonist domain, a receptor antagonist domain, a receptor partial agonist domain, a ρ3-arrestin-biased agonist domain, Gs-biased agonist, Gi-biased agonist, Gq-biased agonist, a caveolae-mediated endocytosis trigger, a clathrin-mediated endocytosis trigger, a lysosomal trigger, a late endosome trigger, a “long recycling” endosome trigger, a “short recycling” endosome trigger, an early endosome trigger, a Rab-mimetic endosomal sorting protein, a biomimetic domain, a cell-mimetic domain, polyethylene glycol (PEG), poly(sarcosine), a N-(2-Hydroxypropyl) (HPMA) linker, a HPMA terminal sequence, a biodegradable polymer, an endosomolytic peptide sequence, a viral peptide sequence, a nuclear trafficking sequence, a microtubule-binding sequence, a histone-derived sequence, or a subcellular trafficking sequence.

18. The composition of claim 12, wherein the functional domain comprises a cell-targeting functional motif selected from the group consisting of an antibody, a single-chain variable fragment (ScFv), an aptamer, a peptoid, a Polymer, a lipid, a polysaccharide, a subcellular cell-targeting motif, an extracellular cell-targeting motif, and a multi-domain sequence.

19. (canceled)

20. The composition of claim 12, wherein the functional domain comprises a cell-penetrating motif selected from the group consisting of a p(asp)[DET], a cationic polymer, poly(L-arginine) (PLR), poly(L-lysine) (PLK), a cationic-rich sequence, a histone, and a cell penetrating peptide (CPP).

21. (canceled)

22. The composition of claim 12, further comprising:
at least one moiety comprising a modified protein, the modified protein comprising:
an anchor, wherein the anchor is a cationic anchor or an anionic anchor designed to interact with a binding patch and wherein the interaction between the anchor and the binding patch may be hydrophilic, hydrophobic, electrostatic, covalent, or non-covalent; and
at least one linker, wherein at least one linker is a terminal linker coupled to the anchor.

23. (canceled)

24. (canceled)

25. The composition of claim 12, wherein the linker is (i) a linker selected from the group consisting of a polyethylene glycol (PEG), N-(2-Hydroxypropyl) methacrylamide (HPMA), poly(sarcosine), a poly(hydrophilic) polymer, a poly(hydrophobic) polymer, a poly(charged) polymer, a charge-switching polymer, a rigid domain, flexible domain, and an aliphatic domain, or (ii) a multi-domain linker having at least one domain selected from the group consisting of a polyethylene glycol (PEG), N-(2-Hydroxypropyl) methacrylamide (HPMA), poly(sarcosine), a poly(hydrophilic) polymer, a poly(hydrophobic) polymer, a poly(charged) polymer, a charge-switching polymer, a rigid domain, flexible domain, and an aliphatic domain.

26. (canceled)

27. The composition of claim 25, wherein the nanoparticle seed substrate comprises a protein selected from the group consisting of Cas9, CasX, CasY, Cpf1, Cas13, MAD7, Rad51, Rad54, transcription activator-like (TAL) effectors (TALEs), TALE nucleases (TALENs), zinc-finger proteins (ZFPs), zinc-finger nucleases (ZFNs), DNA-guided polypeptides, Natronobacterium gregoryi Argonaute (NqAqo), transposons, piggyBac, sleeping beauty, Tc1/mariner, Tol2, PIF/harbinger, hAT, mutator, merlin, transib, helitron, maverick, frog prince, minos, Himar1, meganucleases, I-SceI, I-CeuI, I-CreI, I-DmoI, I-ChuI, I-DirI, I-FlmuI, I-FlmuII, I-AniI, I-SceIV, I-CsmI, I-PanI, I-PanII, I-PanMI, I-ScelI, I-PpoI, I-SceIII, I-LtrI, I-GpiI, I-GZeI, I-OnuI, I-HieMI, I-MsoI, I-TevI, I-TevlI, I-TevIII, PI-MleI, PI-MtuI, PI-PspI, PI-Tli I, PI-Tli II, PI-SceV, megaTALs, SCF, BCL-XL, Foxp3, HoxB4, and SiRT6.

28.-31. (canceled)

32. The composition of claim 27, further comprising:
the nanoparticle seed substrate comprising a modified surface-exposed residue, and
at least one payload or functional domain coupled to the nanoparticle seed substrate through covalent bonding or complementarity with a PNA, MNA, LNA, RNA, DNA, charged sequence, aptamer sequence, or other polymer with binding affinity for the payload or functional domain, wherein incorporation of the payload or functional domain leads to a stapling conjugation to a modified-surface exposed residue on the nanoparticle seed substrate.

33. The composition of claim 27, further comprising at least one payload or functional domain coupled to the nanoparticle seed substrate by a protein that is a wildtype protein or a chimeric protein acting as a binding element.

34. (canceled)

35. The composition of claim 33, wherein the wildtype protein or the chimeric protein is a DNA-binding protein or an RNA-binding protein.

36.-40. (canceled)

41. A method for predictive modeling of a self-assemblable nanoparticle, comprising:
generating a three-dimensional model of a nanoparticle seed substrate having a zwitterionic three-dimensional surface comprising a plurality of cationic and a plurality of anionic binding patches;
generating a Poisson-Boltzmann electrostatic surface charge plot of the nanoparticle seed substrate to overlay on the three-dimensional model; and
using the overlay on the three-dimensional model to charge-tune the nanoparticle seed substrate by
(1) identifying one or more surface-exposed residues within a binding patch for modification, wherein the one or more surface-exposed residues are not catalytically active, and where the one or more surface-exposed residues are not required for activity of the protein with a binding substrate, and
(2) using the overlay on the three-dimensional model to simulate the addition of a plurality of moieties capable of interacting with the binding patches to the nanoparticle seed substrate to perform charge surface engineering of the nanoparticle seed substrate.

42. The method of claim 41, wherein the nanoparticle seed substrate has a zwitterionic charge ratio defined by the number of the cationic binding patches to the number of anionic binding patches (+/−), and further comprising modifying the zwitterionic charge ratio, wherein modifying the zwitterionic charge ratio generates a zwitterionic charge-tuned three-dimensional surface.

43. (canceled)

44. The method of claim 41, wherein at least one of the plurality of moieties comprises a modified protein comprising an anchor, wherein the anchor is a cationic anchor or an anionic anchor designed to interact with a binding patch and wherein the interaction between the anchor and the binding patch may be hydrophilic, hydrophobic, electrostatic, covalent, or non-covalent; and at least one linker, wherein at least one linker is a terminal linker coupled to the anchor.

45.-66. (canceled)


SEQUENCE LISTING (88 sequences)

SEQ ID NO: 1 - protein, 65 aa, synthetic construct
  REGION 1..65: note = Anchor-linker-ligand
  REGION 1..26: note = MISC_FEATURE - Histone H2AX [119-143]
  REGION 27..30: note = MISC_FEATURE - Cleavable domain
  REGION 31..40: note = MISC_FEATURE - Linker domain
  REGION 41..44: note = MISC_FEATURE - Cleavable domain
  REGION 45..65: note = MISC_FEATURE - E-selectin ligand domain
KKRTSATVGPKAPSGGKKATQASQEYFKFLGGGGSGGGGSFKFLMIASQFLSALTLVLLIKESGA

SEQ ID NO: 2 - protein, 61 aa, synthetic construct
  REGION 1..61: note = Anchor-linker-ligand
  REGION 1..26: note = MISC_FEATURE - Histone H2AX [119-143]
  REGION 27..30: note = MISC_FEATURE - Cleavable domain
  REGION 31..40: note = MISC_FEATURE - Linker domain
  REGION 41..61: note = MISC_FEATURE - E-selectin ligand domain
KKRTSATVGPKAPSGGKKATQASQEYFKFLGGGGSGGGGSMIASQFLSALTLVLLIKESGA

SEQ ID NO: 3 - protein, 4 aa, synthetic construct
  REGION 1..4: note = Cleavable domain
FKFL

SEQ ID NO: 4 - protein, 10 aa, synthetic construct
  REGION 1..10: note = Linker domain
GGGGSGGGGS

SEQ ID NO: 5 - protein, 21 aa, synthetic construct
  REGION 1..21: note = E-selectin ligand domain
MIASQFLSALTLVLLIKESGA

SEQ ID NO: 6 - protein, 9 aa, synthetic construct
  REGION 1..9: note = Linker domain
GAPGAPGAP

SEQ ID NO: 7 - protein, 15 aa, synthetic construct
  REGION 1..15: note = IL2 (31-45)
YKNPKLTRMLTFKFY

SEQ ID NO: 8 - protein, 13 aa, synthetic construct
  REGION 1..13: note = IL2 (61-73)
EELKPLEEVLNLA

SEQ ID NO: 9 - protein, 5 aa, synthetic construct
  REGION 1..5: note = Random coiled sequence
KKKRK

SEQ ID NO: 10 - protein, 9 aa, synthetic construct
  REGION 1..9: note = Random coiled sequence
KKAHHHHHH

SEQ ID NO: 11 - protein, 7 aa, synthetic construct
  REGION 1..7: note = Random coiled sequence
RRRRRRR

SEQ ID NO: 12 - protein, 9 aa, synthetic construct
  REGION 1..9: note = Random coiled sequence
RRRRRRRRR

SEQ ID NO: 13 - protein, 26 aa, synthetic construct
  REGION 1..26: note = Histone H2AX [119-143]
KKRTSATVGPKAPSGGKKATQASQEY

SEQ ID NO: 14 - protein, 22 aa, synthetic construct
  REGION 1..22: note = Representative Gly-X-Y domain
GAPGPSGARGERGFPGERGVQG

SEQ ID NO: 15 - protein, 589 aa, Homo sapiens
WSYNTSTEAMTYDEASAYCQQRYTHLVAIQNKEEIEYLNSILSYSPSYYWIGIRKVNNVWVWVGTQKPLTEEAKNWAPGEPNNRQKDEDCVEIYIKREKDVGMWNDERCSKKKLALCYTAACTNTSCSGHGECVETINNYTCKCDPGFSGLKCEQIVNCTALESPEHGSLVCSHPLGNFSYNSSCSISCDRGYLPSSMETMQCMSSGEWSAPIPACNVVECDAVTNPANGFVECFQNPGSFPWNTTCTFDCEEGFELMGAQSLQCTSSGNWDNEKPTCKAVTCRAVRQPQNGSVRCSHSPAGEFTFKSSCNFTCEEGFMLQGPAQVECTTQGQWTQQIPVCEAFQCTALSNPERGYMNCLPSASGSFRYGSSCEFSCEQGFVLKGSKRLQCGPTGEWDNEKPTCEAVRCDAVHQPPKGLVRCAHSPIGEFTYKSSCAFSCEEGFELHGSTQLECTSQGQWTEEVPSCQVVKCSSLAVPGKINMSCSGEPVFGTVCKFACPEGWTLNGSAARTCGATGHWSGLLPTCEAPTESNIPLVAGLSAAGLSLLTLAPFLLWLRKCLRKAKKFVPASSCQSLESDGSYQKPSYIL

SEQ ID NO: 16 - protein, 294 aa, Homo sapiens
WTYHYSEKPMNWQRARRFCRDNYTDLVAIQNKAEIEYLEKTLPFSRSYYWIGIRKIGGIWTWVGTNKSLTEEAENWGDGEPNNKKNKEDCVEIYIKRNKDAGKWNDDACHKLKAALCYTASCQPWSCSGHGECVEIINNYTCNCDVGYYGPQCQFVIQCEPLEAPELGTMDCTHPLGNFSFSSQCAFSCSEGTNLTGIEETTCGPFGNWSSPEPTCQVIQCEPLSAPDLGIMNCSHPLASFSFTSACTFICSEGTELIGKKKTICESSGIWSNPSPICQKLDKSFSMIKEGDYN

SEQ ID NO: 17 - protein, 7 aa, synthetic construct
  REGION 1..7: note = Predicted binding domain
LPYYSSY

SEQ ID NO: 18 - protein, 7 aa, synthetic construct
  REGION 1..7: note = Predicted binding domain
LSYSPSY

SEQ ID NO: 19 - protein, 20 aa, synthetic construct
  REGION 1..20: note = Predicted binding domain
CVEIYIKSPSAPGKWNDEHC

SEQ ID NO: 20 - protein, 19 aa, synthetic construct
  REGION 1..19: note = Predicted binding domain
CVEIYIKREKDVGMWDERC

SEQ ID NO: 21 - protein, 18 aa, synthetic construct
  REGION 1..18: note = SCF (39-56)
LPSHCWISEMVVQLSDSL

SEQ ID NO: 22 - protein, 14 aa, synthetic construct
  REGION 1..14: note = PfRH5 (346-357)
YNNNFCNTNGIRYH

SEQ ID NO: 23 - protein, 9 aa, synthetic construct
  REGION 1..9: note = PfRH5 (200-208)
YGKYIAVDA

SEQ ID NO: 24 - protein, 10 aa, synthetic construct
  REGION 1..10: note = PfRH5 (444-453)
LNIWRTFQKD

SEQ ID NO: 25 - protein, 8 aa, synthetic construct
  REGION 1..8: note = SIRPa binding domain
  VARIANT 7: note = Optional I
  VARIANT 8: note = Optional Q
LIPVGPXX

SEQ ID NO: 26 - protein, 6 aa, synthetic construct
  REGION 1..6: note = SIRPa binding domain
  VARIANT 1: note = Optional F
  VARIANT 2: note = Optional R
XXKGSP

SEQ ID NO: 27 - protein, 4 aa, synthetic construct
  REGION 1..4: note = SIRPa binding domain
TKRE

SEQ ID NO: 28 - protein, 15 aa, synthetic construct
  REGION 1..15: note = Anchor-ligand
LIPVGPIGGFRKGSP

SEQ ID NO: 29 - protein, 12 aa, synthetic construct
  REGION 1..12: note = Anchor-ligand
LIPVGPIGGNQK

SEQ ID NO: 30 - protein, 24 aa, synthetic construct
  REGION 1..24: note = Anchor-ligand
LIPVGPIQWFRGAGPARELIYNQK

SEQ ID NO: 31 - protein, 20 aa, synthetic construct
  REGION 1..20: note = Anchor-ligand
NQKEGHFPRVTTVSESTKRE

SEQ ID NO: 32 - protein, 21 aa, synthetic construct
  REGION 1..21: note = Anchor-ligand
LIPVGPIGGNQKGGGGSTKRE

SEQ ID NO: 33 - protein, 41 aa, synthetic construct
  REGION 1..41: note = Anchor-ligand
LIPVGPIQWFRGAGPARELIYNQKEGHFPRVTTVSESTKRE

SEQ ID NO: 34 - protein, 14 aa, synthetic construct
  REGION 1..14: note = Predicted binding sequence
LIPVGPGGFRKGSP

SEQ ID NO: 35 - protein, 4 aa, synthetic construct
  REGION 1..4: note = gp120 binding domain
NAKT

SEQ ID NO: 36 - protein, 4 aa, synthetic construct
  REGION 1..4: note = gp120 binding domain
SGGD

SEQ ID NO: 37 - protein, 8 aa, synthetic construct
  REGION 1..8: note = gp120 binding domain
WQKVGKAM

SEQ ID NO: 38 - protein, 11 aa, synthetic construct
  REGION 1..11: note = gp120 binding domain
RSVNFTDNAKT

SEQ ID NO: 39 - protein, 12 aa, synthetic construct
  REGION 1..12: note = H2AX catalytic domain
KKATQASEPQEY

SEQ ID NO: 40 - protein, 339 aa, Homo sapiens
MAMQMQLEANADTSVEEESFGPQPISRLEQCGINANDVKKLEEAGFHTVEAVAYAPKKELINIKGISEAKADKILAEAAKLVPMGFTTATEFHQRRSEIIQITTGSKELDKLLQGGIETGSITEMFGEFRTGKTQICHTLAVTCQLPIDRGGGEGKAMYIDTEGTFRPERLLAVAERYGLSGSDVLDNVAYARAFNTDHQTQLLYQASAMMVESRYALLIVDSATALYRTDYSGRGELSARQMHLARFLRMLLRLADEFGVAVVITNQVVAQVDGAAMFAADPKKPIGGNIIAHASTTRLYLRKGRGETRICKIYDSPCLPEAEAMFAINADGVGDAKD

SEQ ID NO: 41 - protein, 1368 aa, Streptococcus pyogenes [sequence omitted]

SEQ ID NO: 42 - protein, 1368 aa, synthetic construct
  REGION 1..1368: note = SpCas9 mutant
MDKKYSIGLDIGTNSVGWAVITDEYDVPSDDFKVLGNTDEHSIDDDLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLDLAHMIKFRGHFLIEGDLNPDNSDVDDLFIELVETYNELFEENPINADDVDAKAILSARLSKSRRLENLIAQLPGEDDNGLFGNGIALSLGLTPNFDSNFDLAEDAKLQLSKDTYDDDLDNLLDQIGDQYADLFLAAKNLSDAILLSDILEVNTEETDAPLSASMIKRYDEHHQDLTLLKALVRQQLPEKYKEIFFDQSKNGYAGYIDGGASQEEFYKFIDPILEDMDGTEELLVDLNREDLLRKQRTFDNGSIPHQIHLGELEAILEEQEDFYPFLKDNEEKIEKILTFRIPYYVGPLARGNSRFAWMTRKSEETITPWNFEEVVDKGASAQSFIERMTNFDKNLPNEKVLPKHSLLYEYFTVYNELTKVDYVTEGMEDPAFLSGEQKKAIVDLLFKTNRKVTVKQLKEDYFKKIECFDSVEISGVEDRFNASLGTYEDLLKIIDDDDFLDNEENEDIGEDIVLTLTLFEDREMIEERLKTYAHLFDDDVMKQLDRREYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGEDMYVDQELDINELSDYDVDHIVPQSFLKDDSIDNKVLTRSDKNRGKSDNVPDEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 43 - protein, 14 aa, synthetic construct
  REGION 1..14: note = Modified IL2 (31-44)
YKNXKLTRMLTXKF

SEQ ID NO: 44 - protein, 13 aa, synthetic construct
  REGION 1..13: note = Modified IL2 (61-73)
EELXXLEEVLNXA

SEQ ID NO: 45 - protein, 9 aa, synthetic construct
  REGION 1..9: note = Ligand
YTSRLHSGV

SEQ ID NO: 46 - protein, 9 aa, synthetic construct
  REGION 1..9: note = Ligand
LGLRGHSGV

SEQ ID NO: 47 - protein, 9 aa, synthetic construct
  REGION 1..9: note = Ligand
LLGRGHSGV

SEQ ID NO: 48 - protein, 9 aa, synthetic construct
  REGION 1..9: note = Ligand
GLLRGHSGV

SEQ ID NO: 49 - protein, 147 aa, synthetic construct
  REGION 1..147: note = Adenosine deaminase (9-155)
RQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGD

SEQ ID NO: 50 - protein, 147 aa, synthetic construct
  REGION 1..147: note = dCpf1-crRNA-DNA complex
EGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTTEH

SEQ ID NO: 51 - protein, 27 aa, synthetic construct
  REGION 1..27: note = CPP 2819
VGALAVVVWLWLWLWAGSGPKKKRKVC

SEQ ID NO: 52 - protein, 33 aa, synthetic construct
  REGION 1..33: note = CPP 2845
KKWALLALALHHLAHLALHLALALKKAHHHHHH

SEQ ID NO: 53 - protein, 34 aa, synthetic construct
  REGION 1..34: note = CPP 2906
YGRKKRRQRRRGLFGAIAGFIENGWEGMIDGWYG

SEQ ID NO: 54 - protein, 25 aa, synthetic construct
  REGION 1..25: note = CPP 2926
RFTFHFRFEFTFHFEGGGRRRRRRR

SEQ ID NO: 55 - protein, 25 aa, synthetic construct
  REGION 1..25: note = CPP 2946
LAQLLAQLLAQLGGGGRRRRRRRRR

SEQ ID NO: 56 - protein, 36 aa, synthetic construct
  REGION 1..36: note = CPP 2959
CGGGARKKAAKAARKKAAKAARKKAAKAARKKAAKA

SEQ ID NO: 57 - protein, 70 aa, synthetic construct
  REGION 1..70: note = Anchor-linker-ligand
KKRTSATVGPKAPSGGKKATQASQEYFKFLGGGGSGGGGSGGGGSFKFLMIASQFLSALTLVLLIKESGA

SEQ ID NO: 58 - protein, 75 aa, synthetic construct
  REGION 1..75: note = Anchor-linker-ligand
KKRTSATVGPKAPSGGKKATQASQEYFKFLGGGGSGGGGSGGGGSGGGGSFKFLMIASQFLSALTLVLLIKESGA

SEQ ID NO: 59 - protein, 63 aa, synthetic construct
  REGION 1..63: note = Anchor-linker-ligand
KKRTSATVGPKAPSGGKKATQASQEYFKFLGAPGAPGAPFFLMIASQFLSALTLVLLIKESGA

SEQ ID NO: 60 - protein, 70 aa, synthetic construct
  REGION 1..70: note = Anchor-linker-ligand
KKRTSATVGPKAPSGGKKATQASQEYFKFLGAPGAPGAPGAPGAPFKFLMIASQFLSALTLVLLIKESGA

SEQ ID NO: 61 - protein, 74 aa, synthetic construct
  REGION 1..74: note = Anchor-linker-ligand
KKRTSATVGPKAPSGGKKATQASQEYFKFLGAPGAPGAPGAPGAPGAPFFKFLMIASQFLSALTLVLLIKESGA

SEQ ID NO: 62 - protein, 77 aa, synthetic construct
  REGION 1..77: note = Anchor-linker-ligand
KKRTSATVGPKAPSGGKKATQASQEYFKFLGAPGPSGARGERGFPGERGVQGFKFLMIASQFLSALTLVLLIKESGA

SEQ ID NO: 63 - protein, 61 aa, synthetic construct; identical to SEQ ID NO: 2
  REGION 1..61: note = Anchor-linker-ligand

SEQ ID NO: 64 - protein, 66 aa, synthetic construct
  REGION 1..66: note = Anchor-linker-ligand
KKRTSATVGPKAPSGGKKATQASQEYFKFLGGGGSGGGGSGGGGSMIASQFLSALTLVLLIKESGA

SEQ ID NO: 65 - protein, 71 aa, synthetic construct
  REGION 1..71: note = Anchor-linker-ligand
KKRTSATVGPKAPSGGKKATQASQEYFKFLGGGGSGGGGSGGGGSGGGGSMIASQFLSALTLVLLIKESGA

SEQ ID NO: 66 - protein, 60 aa, synthetic construct
  REGION 1..60: note = Anchor-linker-ligand
KKRTSATVGPKAPSGGKKATQASQEYFKFLGAPGAPGAPMIASQFLSALTLVLLIKESGA

SEQ ID NO: 67 - protein, 66 aa, synthetic construct
  REGION 1..66: note = Anchor-linker-ligand
KKRTSATVGPKAPSGGKKATQASQEYFKFLGAPGAPGAPGAPGAPMIASQFLSALTLVLLIKESGA

SEQ ID NO: 68 - protein, 69 aa, synthetic construct
  REGION 1..69: note = Anchor-linker-ligand
KKRTSATVGPKAPSGGKKATQASQEYFKFLGAPGAPGAPGAPGAPGAPMIASQFLSALTLVLLIKESGA

SEQ ID NO: 69 - protein, 73 aa, synthetic construct
  REGION 1..73: note = Anchor-linker-ligand
KKRTSATVGPKAPSGGKKATQASQEYFKFLGAPGPSGARGERGFPGERGVQGMIASQFLSALTLVLLIKESGA

SEQ ID NO: 70 - protein, 33 aa, synthetic construct
  REGION 1..33: note = c6bk4a
VGPKAPSGGKKATQASQEYFKFLGGGGSGGGGS

SEQ ID NO: 71 - protein, 16 aa, synthetic construct
  REGION 1..16: note = d2a5yb3
GGSGGGGSMIASQFLS

SEQ ID NO: 72 - protein, 8 aa, synthetic construct
  REGION 1..8: note = Linker domain
GGGGSGGG

SEQ ID NO: 73 - protein, 22 aa, synthetic construct
  REGION 1..22: note = c1p68A
FLGGGGSGGGGSMIASQFLSAL

SEQ ID NO: 74 - protein, 9 aa, synthetic construct
  REGION 1..9: note = c1psmA
KKATQASQE

SEQ ID NO: 75 - protein, 20 aa, synthetic construct
  REGION 1..20: note = c2i7uA
SQEYFKFLGGGGSGGGGSMI

SEQ ID NO: 76 - protein, 9 aa, synthetic construct
  REGION 1..9: note = Linker domain
GGGSGGGGS

SEQ ID NO: 77 - protein, 18 aa, synthetic construct
  REGION 1..18: note = c21qhB
GGGSGGGGSMIASQFLSA

SEQ ID NO: 78 - protein, 7 aa, synthetic construct
  REGION 1..7: note = Linker domain
GGGSGGG

SEQ ID NO: 79 - protein, 21 aa, synthetic construct
  REGION 1..21: note = c2m86A
SGGKKATQASQEYFKFLGGGG

SEQ ID NO: 80 - protein, 18 aa, synthetic construct
  REGION 1..18: note = c2n5dA
GGSGGGGSMIASQFLSAL

SEQ ID NO: 81 - protein, 14 aa, synthetic construct
  REGION 1..14: note = c4dzoA
FLSALTLVLLIKES

SEQ ID NO: 82 - protein, 16 aa, synthetic construct
  REGION 1..16: note = c4zdtB
GSMIASQFLSALTLVL

SEQ ID NO: 83 - protein, 12 aa, synthetic construct
  REGION 1..12: note = c5zcsE/c5zcsF
TQASQEYFKFLG

SEQ ID NO: 84 - protein, 13 aa, synthetic construct
  REGION 1..13: note = c6musB
QASQEYFKFLGGG

SEQ ID NO: 85 - protein, 10 aa, synthetic construct
  REGION 1..10: note = d1k47a2/d1kkha2/k1kvka2
GSGGGGSMIA

SEQ ID NO: 86 - protein, 185 aa, Homo sapiens
TGPCTNVSTVQCTHGIRPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCGGEFFYCNSTQLFNSTWFNSTWSTKGSNNTEGSDTITLPCRIKQIINMWQKVGKAMYAPPISGQIRCSSNI

SEQ ID NO: 87 - protein, 133 aa, Homo sapiens [sequence omitted]

SEQ ID NO: 88 - protein, 34 aa, Homo sapiens
DPELCDDDPPEIPHATFKAMAYKEGTMLNCECKR